(19) United States
(12) Patent Application Publication
(10) Pub. No.: US 2011/0223185 A1
(43) Pub. Date: Sep. 15, 2011

(54) CHIMERIC HEPATITIS C VIRUS ANTIGENS FOR ELICITING AN IMMUNE RESPONSE

(76) Inventors: Rajan George, Edmonton (CA); Lorne Tyrrell, Edmonton (CA); Antoine Noujaim, Edmonton (CA); Dakun Wang, Edmonton (CA); Allan Ma, Edmonton (CA); Bruce Motyka, Edmonton (CA); Bruce Darryl Hirsche, Edmonton (CA)

(21) Appl. No.: 13/110,493

(22) Filed: May 18, 2011

Related U.S. Application Data
(63) Continuation of application No. 12/089,872, filed on Apr. 29, 2009, now abandoned.
(60) Provisional application No. 60/726,701, filed on Oct. 13, 2005.

(51) Int. Cl. A61K 39/395; C12P 21/00; C12N 5/071; C07K 19/00; C07H 21/00; C07H 21/04; C12N 15/63; C12N 5/10; A61P 37/04; C12N 1/00; A61P 31/14

(52) U.S. Cl. 424/178.1; 435/69.3; 435/375; 530/391.7; 530/391.9; 536/23.4; 435/320.1; 435/325; 435/419; 435/243

(57) ABSTRACT

Disclosed herein are chimeric antigens, comprising an hepatitis C virus (HCV) antigen and a Fc fragment of an immunoglobulin for eliciting an immune response against said antigen. The immune response is enhanced by presenting the host immune system with an immune response domain (HCV antigen from HVC core, envelope, or non-structural protein fragments) and a target binding domain (an Fc fragment). By virtue of the target binding domain, antigen presenting cells internalize and process the chimeric antigens for antigen presentation, thereby eliciting both a humoral and cellular immune response.

### TECHNICAL FIELD

The present invention relates to chimeric antigens (e.g., fusion proteins) for targeting and activating antigen presenting cells (APCs) to elicit cellular and humoral immune responses. In particular, the invention describes compositions and methods that contain or use one or more chimeric antigens that contain one or more pre-selected Hepatitis C Virus (HCV) antigen(s), and an immunoglobulin fragment, wherein the chimeric antigen is capable of binding and activating APCs, especially dendritic cells, which process and perform antigen presentation to elicit cellular and humoral immune responses.

### BACKGROUND

More than 170 million people worldwide are chronic carriers of HCV [Delwaide et al. (2000) Rev. Med. Liege 55:337-340] There is neither a prophylactic nor a therapeutic vaccine currently available for HCV. The route of infection is via blood and other body fluids and over 70% of patients become chronic carriers of the virus. Persistent infection results in chronic active hepatitis which may lead to progressive liver disease [Alter et al, (1999) N. Engl. J. Med. 341:556-562]. Presently, the only therapy for hepatitis C infection is interferon-I (IFN-1) and Ribavirin. However, this therapy is expensive, has substantial side effects, and is effective in only approximately 50% of a selected group of patients. Therapeutic vaccines that enhance host immune responses to zo eliminate chronic HCV infection will be a major advancement in the treatment of this disease.

The immune system plays a key role in the outcome of an HCV infection. Most individuals that are exposed to HCV mount a broad strong and multi-antigen-specific CD4+ (regulatory) and CD8+ (cytotoxic) T cell response to the virus. These individuals develop only a self-limited infection. However, in some individuals exposed to HCV, a weak or undetectable and narrowly focused immune response results in chronic infection.

HCV is a member of the flaviviridae family of RNA viruses. The HCV genome is a positive sense single stranded RNA molecule of approximately 9.5 Kb that encodes a single polyprotein which is cleaved into individual proteins catalyzed by host and viral proteases to produce three structural proteins (core, E1, E2), p7 protein and 6 non-structural proteins (NS2, NS3, NS4A, NS4B, NS5A, NS5B) [Hijikkata et al. (1991) Proc. Natl. Acad. Sci. USA 88:5547-5551]. The NS3 protein is the viral serine protease involved in the proteolytic processing of the non-structural proteins [Bartenschlager et al. (1993) J. Virol. 67: 3835-3844].

The mechanism by which the virus evades the host immune machinery is not clearly established [Shoukry et al. (2004) Ann. Rev. Microbiol. 58:391-424]. Several HCV proteins have been implicated in the immune evasion mechanism. These include: NS5A, suggested to induce the production of IL-8 which inhibits the IFN-induced antiviral response [Polyak at al. (2001) J. Virol. 75: 6095-6106] and to inhibit the cellular IFN-γ-induced PKR protein kinase, thus inhibiting antiviral immune responses [Tan et al. (2001) Virol 284: 1-12]; Core and NS3, suggested to inhibit DC differentiation [Dolganiuc et al. (2003) J. Immunol. 170:5615-5624]; and Core and E1 [Sarobe et al. (2002) J. Virol. 77:10862-10871; and Grakoui et al. (2003) Science 302:659-662] suggested to modulate T cell responses by modulating DC maturation; and finally the lack of memory T cell help [Shoji et al. (1999) Virology 254: 315-323].

### SUMMARY

The present invention pertains to compositions and methods for targeting and activating APCs, one of the first steps in eliciting an immune response. The compositions of the present invention include a novel class of molecules (hereinafter designated as “chimeric antigens”) that include an immune response domain (IRD), for example a recombinant protein, linked to a target binding domain (TBD), for example, an antibody fragment portion. More specifically, the chimeric antigens are molecules that couple viral antigens, such as Hepatitis C Core, envelope proteins such E1 and E2, or non-structural so proteins, to an immunoglobulin fragment, such as a murine immunoglobulin G Fc fragment. In some embodiments, the antibody fragment is a xenotypic antibody fragment.

The compositions and methods of the present invention are useful for targeting and activating APCs. The compositions and methods of the present invention are useful for inducing cellular and/or humoral host immune responses against any viral antigen associated with HCV. The invention includes therapeutic vaccines for the treatment of chronic HCV infections as well as prophylactic vaccines for the prevention of HCV infections.

One or more embodiments of the present invention include one or more chimeric antigens suitable for initiating an immune response against HCV. In these embodiments of the invention, selected HCV antigens are linked to fragments of antibodies. The resulting chimeric antigens are capable of targeting and activating APCs, such as dendritic cells.

The present invention also includes methods for cloning DNA constructs encoding fusion proteins and producing fusion proteins in a heterologous expression system. In preferred embodiments of the invention, the cloning and production methods introduce unique post-translational modifications including, but not limited to glycosylation (e.g., mannosylation) of the expressed fusion proteins.

In order to provide efficient presentation of the antigens, the inventors have developed a novel viral antigen-murine monoclonal antibody Fc fragment fusion protein. This molecule, by virtue of the Fc fragment, is recognized at a high efficiency via specific receptors by APCs (e.g., dendritic cells), the fusion protein is processed and peptide epitopes from the viral antigen are presented as complexes with Major Histocompatibility Complex (MHC) Class 1. This processing and antigen presentation results in the up-regulation of the response by cytotoxic T-lymphocytes, resulting in the elimination of virus-infected cell population. In addition, due to antigen presentation by MHC Class II molecules and activation of helper T cells, a Immoral response can be induced against the viral antigen that will help prevent and/or eliminate viral infection.

The chimeric nature of the molecule helps to target the antigen to the proper antigen-presenting cells (e.g., dendritic cells), making it a unique approach in the therapy of chronic infectious diseases by specifically targeting the APC receptors. This is useful for developing therapeutic vaccines to treat chronic Hepatitis C infections.

The administration of these chimeric fusion proteins can elicit a broad immune response from the host, including both cellular and humoral responses. Thus, they can be used as therapeutic vaccines to treat subjects that are immune tolerant to a HCV infection.

More specifically, the invention features a chimeric antigen for eliciting an immune response, the chimeric antigen containing an immune response domain and a target binding domain, the immune response domain containing a hepatitis C(HCV) antigen and the target binding domain containing an antibody fragment. The antibody fragment can be a xenotypic antibody fragment. The chimeric antigen can elicit a humoral immune response, a cellular immune response, or a both humoral immune response and a cellular immune response. In addition, the chimeric antigen can elicit a Th1 immune response, a Th2 immune response or both a Th1 and a Th2 immune response. The immune response can be an in vivo or an ex vivo immune response. The immune response domain can contain more than one protein; it can, for example, contain one or more immunogenic portions of one or more proteins that include, for example, a HCV Core (1-191) protein, a HCV Core (1-177) protein, a HCV p7 protein, a HCV E1 protein, a HCV E2 protein, a HCV E1-E2 protein, a HCV NS3 protein, a HCV NS4B protein, or a HCV NS5A protein. The target binding domain can be capable of binding to an antigen presenting cell (APC). The antibody fragment can be a Fc fragment. The chimeric antigen can further comprise one or more of a 6×His tag, a protease cleavage site, and a linker for linking the immune response domain and the target binding domain. The linker can be selected from leucine zippers, biotin bound to avidin, and a covalent peptide linkage. Furthermore, the chimeric antigen can be glycosylated, e.g., mannose glycosylated. The antibody fragment can include an immunoglobulin heavy chain fragment and the immunoglobulin heavy chain fragment can contain a hinge region. In addition, the immunoglobulin heavy chain fragment can contain all or a part of an antibody fragment selected from the group consisting of the C_{H}1, the hinge region, the C_{H}2 domain, and the C_{H}3 domain.

Another embodiment of the invention is a method of delivering an antigen to an antigen presenting cell, the method comprising administering to the antigen presenting cell any of the chimeric antigens disclosed herein. The antigen presenting cell can be a dendritic cell.

The invention also provides a method of activating an antigen presenting cell; the method can involve contacting an antigen presenting cell with a any of the chimeric antigens described herein. The contacting can take place ex vivo or in vivo. It can take place, for example, in a human. The method can include administering to a subject a composition comprising any of the chimeric antigens of the invention, the antigen presenting cell being in the subject. The contacting can result in a humoral immune response, a cellular immune response, or both a humoral immune response and a cellular immune response. The cellular immune response can be one or more of a Th1 response, a Th2 response, and a CTL response. The subject can have, or be likely to have, an immune-treatable condition. The immune-treatable condition can be an acute infection (e.g., an acute viral infection) or it can be a chronic infection (e.g., a chronic viral infection). The chronic infection can be a chronic hepatitis C viral infection. The immune-treatable condition can be a hepatitis C viral infection and the immune response domain can contain one or more antigenic portions of one or more proteins selected from the group consisting of a HCV Core (1-191) protein, a HCV Core (1-177) protein, a HCV E1 protein, a HCV E2 protein, a HCV E1-E2 protein, a HCV P7 protein, a HCV NS3 protein, a HCY NS4B protein, and a HCV NS5A protein. Using the method, the subject can be vaccinated against a viral infection, e.g., prophylactically vaccinated against a viral infection or therapeutically vaccinated against an existing viral infection.

Another aspect of the invention is a method of producing a chimeric antigen. The method can involve: (a) providing a microorganism or a cell, the microorganism or cell containing a polynucleotide that encodes a chimeric antigen; and (b) culturing the microorganism or cell under conditions whereby the chimeric antigen is expressed. The microorganism or cell can be a eukaryotic microorganism or cell. The cell can be a yeast cell, a plant cell or an insect cell. In addition the chimeric antigen can be post-translationally modified to comprise glycosylation, e.g., it can be post-translationally modified to comprise a mannose glycosylation.

Yet another embodiment of the invention is a polynucleotide encoding a chimeric antigen, the polynucleotide containing a first polynucleotide portion encoding an immune response domain and a second polynucleotide portion encoding a target binding domain, the target binding domain containing an antibody fragment. The antibody fragment can be a xenotypic antibody fragment. The polynucleotide can contain, for example, a nucleotide sequence selected from the group consisting of the nucleotide sequences set forth in SEQ ID NOs:39 and 41-51. Moreover, the polynucleotide can encode a chimeric antigen that is at least 90% identical to an entire amino acid sequence selected from the group consisting of the amino acid sequences set forth in SEQ ID NOs:40 and 52-62. The polynucleotide can selectively hybridize under stringent conditions to a polynucleotide having a nucleotide sequence selected from the group consisting of nucleotide sequences set forth in SEQ ID NOs:39 and 41-51. The invention also provides a vector containing any of the polynucleotides disclosed herein, e.g., a vector in which the polynucleotide is operably linked to a transcriptional regulatory element (TRE). In addition, the invention embraces a microorganism or cell containing any of the polynucleotides disclosed herein.

Another embodiment of the invention is an article of manufacture that can contain any of the chimeric antigens disclosed herein and instructions for administering the chimeric antigen to a subject in need thereof.

Yet another aspect of the invention is a pharmaceutical composition containing any of the chimeric antigens disclosed herein and a pharmaceutically acceptable excipient.

Moreover, the invention provides another method of producing a chimeric antigen. The method can involve: (a) providing a microorganism or a cell, the microorganism or cell containing a polynucleotide that encodes a target binding domain-linker molecule, the target-binding domain-linker molecule containing a target binding domain bound to a linker molecule; (b) culturing the microorganism or cell under conditions whereby the target binding domain-linker molecule is expressed; and (c) contacting the target binding domain-linker molecule and an immune response domain under conditions that allow for the binding of the linker to the immune response domain, the binding resulting in a chimeric antigen. The microorganisms or cells, the polynucleotides, the target binding domains, the linker molecules, and the immune response domains can be any of those disclosed herein.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., chimeric antigens for treating or preventing an immune-treatable or condition, will be apparent from the following description, from the drawings and from the claims.

### BRIEF DESCRIPTION OF THE DRAWINGS

FIG. 1 is schematic depiction of a dimerized form of Chimigen3 vaccine. It contains two subunits, each composed of an immune response domain (IRD) and a target binding domain (TBD).

FIGS. 2A and B are depictions of the nucleotide sequence (SEQ ID NO:9) of the ORF (open reading frame) in plasmid pFastBacHTa-TBD and the amino acid sequence zo (SEQ ID NO:10) encoded by the ORF, respectively.

FIGS. 3A and B are depictions of the nucleotide sequence (SEQ ID NO:39) of the ORF in plasmid pFastBacHTa-NS5A-TBD and the amino acid sequence (SEQ ID NO:40) encoded by the ORF, respectively. There is a conserved spontaneous mutation (TTC (Phe) to TYT (Phe)) at the underlined and bolded positions in FIG. 3A and FIG. 3B.

In the nucleotide sequence:
nucleotides 1-3: start codon
nucleotides 13-30: 6×His epitope tag
nucleotides 91-1431: HCV NS5A
nucleotides 1432-1461: linker peptide
nucleotides 1462-2157: TBD
nucleotides 2158-2187: terminal peptide
nucleotides 2188-2190: stop codon
nucleotides 1639-1641: TBD TTC-TTT conserved mutation

In the amino acid sequence:
amino acids 5-10: 6×His epitope tag
amino acids 31-477: HCV NS5A
amino acids 478-487: linker peptide
amino acids 488-719: TBD
amino acids 720-729: terminal peptide

FIGS. 4A and B are depictions of the nucleotide sequence (SEQ ID NO:41) of the ORF in plasmid pFastBacHTa-gp64-NS5A-TBD and the amino acid sequence (SEQ ID NO:52) encoded by the ORF, respectively. There is an artifactual mutation (GAT (Asp) to TAT (Tyr)) and a conserved spontaneous mutation (TTC (Phe) to TTT (Phe)) at the underlined and bolded positions in FIG. 4A and FIG. 4B.

In the nucleotide sequence:
nucleotides 1-3: start codon
nucleotides 1-72: gp64 signal peptide
nucleotides 97-114: 6×His epitope tag
nucleotides 175-1515: HCV NS5A
nucleotides 1516-1545: linker peptide
nucleotides 1546-2241: TBD
nucleotides 2242-2271: terminal peptide
nucleotides 2272-2274: stop codon
nucleotides 61-63: signal peptide GAT to TAT artifactual mutation
nucleotide 1725: TBD TTC to TTT conserved mutation

In the amino acid sequence:
amino acids 1-24: gp64 secretion signal
amino acids 33-38: 6×His epitope tag
amino acids 59-505: HCV NS5A
amino acids 506-515: linker peptide
amino acids 516-747: TBD
amino acids 748-757: terminal peptide
amino acid 21: signal peptide D to Y artifactual mutation

FIGS. 5A and B are depictions of the nucleotide sequence (SEQ ID NO:42) of the ORF in plasmid pPSC12-NS5A-TBD and the amino acid sequence (SEQ ID NO:53) encoded by the ORF, respectively.

FIGS. 6A and B are depictions of the nucleotide sequence (SEQ ID NO:43) of the ORF in plasmid pFastBacHTa-gp64-NS3-TBD and the amino acid sequence (SEQ ID NO:54) encoded by the ORF, respectively. There are two mutations shown by the underlined codons in FIG. 6A and amino acids in FIG. 6B. Upstream to downstream for FIGS. 6A and N-terminal to C-terminal for FIG. 6B, the mutations are: an engineered CGG (Arg) to GCG (Ala) mutation; and a spontaneous CCA (Pro) to GGA (Gly) mutation.

FIGS. 7A and B are depictions of the nucleotide sequence (SEQ ID NO: 44) of the ORF in plasmid pFastBacHTa NS3mut-TBD and the amino acid sequence (SEQ ID NO:55) encoded by the ORF, respectively. There are two mutations shown by the underlined and bolded codons in FIG. 7A and amino acids in FIG. 7B. Upstream to downstream for FIGS. 7A and N-terminal to C-terminal for FIG. 7B, the mutations are: a spontaneous conserved AGG (Arg) to CGG (Arg) mutation; and an engineered COG (Arg) to GCG (Ala) mutation.

In the nucleotide sequence:
nucleotides 1-3: start codon
nucleotides 13-30: 6×His epitope tag
nucleotides 91-1965: HCV NS3mut
nucleotides 1966-1995: linker peptide
nucleotides 1996-2691: TBD
nucleotides 2692-2721: terminal peptide
nucleotides 2722-2724: stop codon
nucleotide 1462-1464: NS3mut AGG to CGG spontaneous mutation
nucleotides 1474-1476: NS3mut CGG to GCG engineered mutation

In the amino acid sequence:
amino acids 5-10: 6×His epitope tag
amino acids 31-655: HCV NS3mut
amino acids 656-665: linker peptide
amino acids 666-897: TBD
amino acids 898-907: terminal peptide
amino acid 488: NS3mut R to R spontaneous mutation
amino acid 492: NS3mut R to A engineered mutation

FIGS. 8A and 13 are depictions of the nucleotide sequence (SEQ ID NO:45) of the ORF in plasmid pFastBacHTa-gp64-NS3mut-TBD and the amino acid sequence (SEQ ID NO:56) encoded by the ORF, respectively. There are three mutations shown by the highlighted codons in FIG. 8A and amino acids in FIG. 8B. Upstream to downstream for FIGS. 8A and N-terminal to C-terminal for FIG. 8B, the mutations are: an artifactual mutation (GAT (Asp) to TAT (Tyr)); a spontaneous conserved AGG (Arg) to CGG (Arg) mutation; and an engineered COG (Arg) to GCG (Ala) mutation.

In the nucleotide sequence:
nucleotides 1-3: start codon
nucleotides 1-72: gp64 signal peptide
nucleotides 97-114: 6×His epitope tag
nucleotides 175-2049: HCV NS3mut
nucleotides 2050-2079: linker peptide
nucleotides 2080-2775: TBD
nucleotides 2776-2805: terminal peptide
nucleotides 2806-2808: stop codon
nucleotides 61-63: signal peptide GAT to TAT artifactual mutation
nucleotides 1546-1548: NS3mut AGG to CGG conserved mutation
nucleotides 1558-1560: NS3mut CGG-GCG engineered mutation

In the amino acid sequence:
amino acids 1-24: gp64 secretion signal
amino acids 33-38: 6×His epitope tag
amino acids 59-683: HCV NS3mut
amino acids 684-693: linker peptide
amino acids 694-925: TBD
amino acids 926-935: terminal peptide
amino acid 21: signal peptide D to Y artifactual mutation
amino acid 520: NS3mut R to A engineered mutation

FIGS. 9A and B are depictions of the nucleotide sequence (SEQ ID NO:46) of the ORF in plasmid pFastBacHTa-gp64 NS3-NS4B-NS5A-TBD and the amino acid sequence (SEQ ID NO:57) encoded by the ORF, respectively. There are four mutations shown by the underlined and bolded codons in FIG. 9A and amino acids in FIG. 9B. Upstream to downstream for FIGS. 9A and N-terminal to C-terminal for FIG. 9B, the mutations are: an artifactual mutation (GAT (Asp) to TAT (Tyr)); an engineered TCG (Ser) to GCG (Ala) mutation; an engineered CGG (Arg) to GCG (Ala) mutation; and a spontaneous CCA (Pro) to GGA (Gly) mutation.

FIGS. 10A and B are depictions of the nucleotide sequence (SEQ ID NO:47) of the ORF in plasmid FastBacHTa-gp64-NS3-NS5A-TBD and the amino acid sequence (SEQ ID NO:58) encoded by the ORF, respectively. There are four mutations shown by the underlined codons in FIG. 10A and amino acids in FIG. 10B. Upstream to downstream for FIGS. 10A and N-terminal to C-terminal for FIG. 10B, the mutations are: an artifactual mutation (GAT (Asp) to TAT (Tyr)); an engineered TCG (Ser) to GCG (Ala) mutation; an engineered CGG (Arg) to GCG (Ala) mutation; and a spontaneous CCA (Pro) to GGA (Gly) mutation.

In the nucleotide sequence:
nucleotides 1-3: start codon
nucleotides 1-72: gp64 signal peptide
nucleotides 97-114: 6×His epitope tag
nucleotides 175-2049: HCV NS3mut
nucleotides 2050-2058: linker peptide
nucleotides 2059-3402: HCV NS5A
nucleotides 3403-3426: linker peptide
nucleotides 3427-4122: TBD
nucleotides 4123-4152: terminal peptide
nucleotides 4153-4155: stop codon
nucleotides 61-63: signal peptide GAT to TAT artifactual mutation
nucleotide 589: NS3mut TCG to GCG engineered mutation
nucleotides 1558-1559: NS3mut CGG to GCG engineered mutation
nucleotides 2050-2052: NS3mut CCA to GGA spontaneous mutation

In the amino acid sequence:
amino acids 1-24: gp64 secretion signal
amino acids 33-38: 6×His epitope tag
amino acids 59-683: HCV NS3
amino acids 684-686: linker peptide
amino acids 687-1134: HCV NS5A
amino acids 1135-1374: TBD
amino acids 1375-1384: terminal peptide
amino acid 21: signal peptide D to Y artifactual mutation
amino acid 197: NS3mut S to A engineered mutation
amino acid 520: NS3mut R to A engineered mutation
amino acid 684: NS3mut P to G spontaneous mutation

FIGS. 11A and B are depictions of the nucleotide sequence (SEQ ID NO:48) of the ORF in plasmid pFastBacHTa HCV core (1-177)-TBD and the amino acid sequence (SEQ ID NO:59) encoded by the ORF, respectively.

FIGS. 12A and B are depictions of the nucleotide sequence (SEQ ID NO:49) of the ORF in plasmid pFastBacHTa-E1-TBD and the amino acid sequence (SEQ ID NO:60) encoded by the ORF, respectively.

FIGS. 13A and B are depictions of the nucleotide sequence (SEQ ID NO:50) of the ORE in plasmid pFastBacHTa E2-TBD and the amino acid sequence (SEQ ID NO:61) encoded by the ORF, respectively.

FIGS. 14A and B are depictions of the nucleotide sequence (SEQ ID NO:51) of the ORF in plasmid pFastBacHTa-E1-E2-TBD and the amino acid sequence (SEQ ID NO:62) encoded by the ORF, respectively.

FIG. 15 is a series of fluorescence flow cytometry (FFC) profiles showing binding, at three different concentrations, of the NS5A Chimigen3 Protein to immature DCs.

FIG. 16 is a pair of bar graphs showing inhibition of binding of the NS5A Chimigen3 Protein to immature DCs by antibodies specific for CD32 and CD206.

FIG. 17 is a series of bar graphs showing the expression of the indicated cell surface markers by mature DC produced as described in the Examples. The data were obtained by FFC and are presented as “% percent positive cells” (top graphs) and “mean fluorescence intensity” (“MFI”) (bottom graphs).

FIGS. 18A-B are three sets of bar graphs showing the proportion of CD69 expressing T cells (FIG. 18A), CD69 expressing CD8⁺ T cells (FIG. 18B), and CD69 expressing CD4⁺ T cells (FIG. 18C) in day 4 cultures of various concentrations of T cells and NS5A Chimigen3 Protein or tetanus toxoid loaded DC. The data were obtained by FFC. 5AC1 and 5AC2: two different preparations of NS5A Chimigen3 Protein.

FIGS. 19A-C are three sets of bar graphs showing the proportion of CFSElo T cells (FIG. 19A), CFSElo CDS⁺ T cells (FIG. 19B), and CFSElo CD4⁺ T cells (FIG. 19C) in day 4 cultures of various concentrations of T cells and NS5A Chimigen3 Protein or tetanus toxoid loaded DC. The data were obtained by FFC. 5AC1 and 5AC2: two different preparations of NS5A Chimigen3 Protein.

FIGS. 20A-C are three sets of bar graphs showing the proportion of CD69 expressing T cells (FIG. 20A), CD69 expressing CD8⁺ T cells (FIG. 20B), and CD69 expressing CD4⁴ T cells (FIG. 20C) in day 7 cultures of various concentrations of T cells and NS5A Chimigen3 Protein or tetanus toxoid loaded DC or phytohemagglutinin (PHA; in FIG. 20A). The data were obtained by FFC. 5AC1 and 5AC2: two different preparations of NS5A Chimigen3 Protein.

FIGS. 21A-C are three sets of bar graphs showing the proportion of CFSElo T cells (FIG. 21A), CFSElo CD8⁺ T cells (FIG. 21B), and CFSElo CD4⁺ T cells (FIG. 21C) in day 7 cultures of various concentrations of T cells and NS5A Chimigen3 Protein or tetanus toxoid loaded DC or PHA (in FIG. 21A). The data were obtained by FFC. 5AC1 and 5AC2: two different preparations of NS5A Chimigen3 Protein.

FIG. 22 is a series of bar graphs showing the proportion of T cells that are blasts in 7 day cultures of various concentrations of T cells and NS5A Chimigen3 Protein or tetanus toxoid loaded DC or PHA. The data were obtained by FFC. 5AC1 and 5AC2: two different preparations of NS5A Chimigen3 Protein.

FIG. 23 is a series of bar graphs showing the expression by matured, antigen-loaded DC of the indicated cell surface markers. The data were obtained by FFC.

FIG. 24 is a pair of bar graphs showing the proportion of T cells that are blasts after three stimulations with matured, antigen loaded DC made as described in the Examples. The data were obtained by (FFC). 5AC: NS5A Chimigen3 Protein.

FIG. 25 is a series of bar graphs showing the proportion of T cells containing intracellular interferon-K (IFN-K) after three stimulations with matured, antigen loaded DC made as described in the Examples. The data were obtained by FFC. 5AC: NS5A Chimigen3 Protein; PMA: phorbol myristic acid; Dulbecco's phosphate buffered saline (DPBS).

FIG. 26 is a pair of bar graphs showing the proportion of CD8⁺ T cells containing intracellular IFN-K after three stimulations with matured, antigen loaded DC made as described in the Examples. The data were obtained by FFC. 5AC: NS5A Chimigen3 Protein.

FIG. 27 is a pair of bar graphs showing the proportion of CD4⁺ T cells containing intracellular IFN-K after three stimulations with matured, antigen loaded DC made as described in the Examples. The data were obtained by FFC. 5AC: NS5A Chimigen3 Protein.

FIG. 28 is a pair of bar graphs showing the proportion of T cells containing intracellular tumor necrosis factor-I (TNF-I) after three stimulations with matured, antigen loaded DC made as described in the Examples. The data were obtained by FFC. 5AC: NS5A Chimigen3 Protein; PMA: phorbol myristic acid; DPBS.

FIG. 29 is a pair of bar graphs showing the proportion of CM⁺ T cells (left graph) and CD4⁺ T cells (right graph) containing intracellular TNF-I after three stimulations with matured, antigen loaded DC made as described in the Examples. The data were obtained by FFC. 5AC: NS5A Chimigen3 Protein.

FIG. 30 is a pair of bar graphs showing the proportion of CD8⁺ T cells expressing the granular proteins GrB (left graph) and Pfn (right graph) after three stimulations with matured, antigen loaded DC made as described in the Examples. The data were obtained by FFC. 5AC: NS5A Chimigen3 Protein.

FIG. 31 is a pair of bar graphs showing the total number of lymphocytes (R1 gated cells) and the proportion of blast cells after three stimulations with matured, antigen loaded DC made as described for in the Examples. The cells were analyzed 6 days after the last stimulation. The data were obtained by FFC. 5AC: NS5A Chimigen3 Protein.

FIG. 32 is a pair of graphs showing the relative proportions of CD69 expressing CD8⁺ T cells (left graph) and CD69 expressing CD4+ T cells (right graph) after three stimulations with matured, antigen loaded DC made as described in the Examples. The cells were analyzed 6 days after the last stimulation. The data were obtained by FFC. 5AC: NS5A Chimigen3 Protein.

FIG. 33 is a pair of bar graphs showing the relative proportions of CD8⁺ T cells (left graph) and CD4⁺ T cells (right graph) having antigen specific T cell receptors (TCR) that bound an EBVpeptide/HLA-A2 tetramer (positive control) or a control tetramer (negative tetramer) after three stimulations with matured, antigen loaded DC made as described in the Examples. The cells from three individual culture wells (corresponding to the three bars in the test and control groups) were analyzed 6 days after the last stimulation. The data were obtained by FFC.

FIG. 34 is a pair of bar graphs showing the relative proportions of CD8⁺ T cells having TCR that bound NS5A peptide/HLA-A2 pentamer after three stimulations (using different numbers of T cells and DC) with matured, antigen loaded DC made as described in the Examples. The cells from three individual culture wells (corresponding to the three bars in the test and control groups) were analyzed 6 days after the last stimulation. The data were obtained by FFC. 5AC: NS5A Chimigen3 Protein.

FIG. 35 is a series of FFC profiles showing binding, at two different concentrations, of the NS3 Chimigen3 Protein to immature DCs.

FIG. 36 is a pair of bar graphs showing inhibition of binding of the NS3 Chimigen3 Protein to immature DCs by antibodies specific for CD32 and CD206.

FIG. 37 is a series of bar graphs showing the proportion of CD69 expressing CD8⁺ T cells (left graphs) and CD69 expressing CD4⁺ T cells (right graphs) in day 4 (top graphs) and day 7 cultures containing NS3 Chimigen3 Protein or tetanus toxoid loaded DC. The data were obtained by FFC. 3C: NS3 Chimigen3 Protein.

FIG. 38 is a series of bar graphs showing the proportion of CFSElo CD8⁺ T cells (left graphs) and CFSElo CD4⁺ T cells (right graphs) in day 4 (top graphs) and day 7 cultures containing NS3 Chimigen3 Protein or tetanus toxoid loaded DC. The data were obtained by FFC. 3C: NS3 Chimigen3 Protein.

FIG. 39 is a pair of bar graphs showing the proportion of T cells that are blasts after three stimulations with matured, antigen loaded DC made as described for the NS5A Chimigen3 Protein in the Examples. The stimulations were performed using two different T cell and two different DC concentrations. The data were obtained by FC. 3C: NS3 Chimigen3 Protein.

FIG. 40 is a series of bar graphs showing the proportion of T cells containing intracellular IFN-K after three stimulations with matured, antigen loaded DC made as described in the Examples. The data were obtained by FFC. 3C: NS3 Chimigen3 Protein.

FIG. 41 is a pair of bar graphs showing the proportion of CD8⁺ T cells (left graph) and CD4⁺ T cells (right graph) containing intracellular IFN-K after three stimulations with matured, antigen loaded DC made as described in the Examples. The data were obtained by FFC. 3C: NS3 Chimigen3 Protein.

FIG. 42 is a pair of bar graphs showing the proportion of CD8⁺ T cells (left graph) and CD4⁺ T cells (right graph) containing intracellular TNF-1 after three stimulations with matured, antigen loaded DC made as described in the Examples. The data were obtained by FFC. 3C: NS3 Chimigen3 Protein.

FIG. 43 is a pair of bar graphs showing the proportion of CD8⁺ T cells expressing the granular proteins GrB (left graph) and Pfn (right graph) after three stimulations with matured, antigen loaded DC made as described in the Examples. The data were obtained by FFC. 3C: NS3 Chimigen3 Protein.

FIG. 44 is a pair of graphs showing the relative proportions of CD69 expressing CD8⁺ T cells (left graph) and CD69 expressing CD4+ T cells (right graph) after three stimulations with matured, antigen loaded DC made as described in the Examples. The cells were analyzed 6 days after the last stimulation. The data were obtained by FFC. 3C: NS3 Chimigen3 Protein.

FIG. 45 is a pair of bar graphs showing the total number of lymphocytes (R1 gated cells) (left graph) and the proportion of blast cells (right graph) after three stimulations with matured, antigen loaded DC made as described in the Examples. The cells were analyzed 6 days after the last stimulation. The data were obtained by FFC. 3C, AS60-1: NS3 Chimigen3 Protein.

FIG. 46 is a pair of bar graphs showing the relative proportions of CD8⁺ T cells having TCR that bound NS3 peptide/HLA-A2 pentamer after three stimulations (using different numbers of T cells and DC) with matured, antigen loaded DC made as described in the Examples. The cells from three individual culture wells (corresponding to the three bars in both the test groups and the control group) were analyzed 5 days after the last stimulation. The data were obtained by FFC. 3C: NS3 Chimigen3 Protein.

FIG. 47 is a series of fluorescence flow cytometry (FFC) profiles showing binding, at three different concentrations, of the HCV Core Chimigen3 Protein to immature DCs.

FIG. 48 is a pair of bar graphs showing inhibition of binding of the HCV Chimigen3 Core Protein to immature DCs by antibodies specific for CD32 and CD206, mannosylated bovine serum albumin (mBSA), and murine IgG fragments.

FIG. 49 is a pair of bar graphs showing the proportion of CD8⁺ T cells (left graph) and CD4⁺ T cells (right graph) containing intracellular IFN-K after three stimulations with matured, antigen loaded DC made as described in the Examples. The data were obtained by FFC. HCV Core-TBD: HCV Core Chimigen3 Protein.

FIG. 50 is a pair of two-dimensional FFC dot plots showing the proportion of CD8⁺ T cells having TCR that bound a HCV Core peptide/HLA-B7 tetramer after three stimulations with DC loaded with the HCV Core Chimigen3 Protein (HCV Core-TBD) (right dot plot) or TBD alone (TBD) (left dot plot).

### DETAILED DESCRIPTION

### A. Overview

Disclosed herein are compositions and methods for eliciting immune responses against antigens. In particular embodiments, the compounds and methods elicit immune responses against antigens that are otherwise recognized by the host as “self” antigens. The immune response is enhanced by presenting the host immune system with a chimeric antigen comprising an immune response domain and a target binding domain, wherein the target binding domain comprises an antibody fragment. By virtue of the target binding domain, APCs internalize, process and present the chimeric antigen, eliciting both humoral and cellular immune responses.

HCV is a member of the flaviviridae family which'can infect humans, resulting in acute and chronic hepatitis, and may result in hepatocellular carcinoma [Hoofnagle (2002) Hepatology 36:S21-S29]. The HCV genome is a 9.6 Kb uncapped positive polarity single stranded RNA molecule and the replication occurs via a negative-strand intermediate [Lindenbach and Rice (2005) Nature 436:933-938]. The HCV genome encodes a single open reading frame that encodes a polyprotein, which is processed to generate the core or capsid protein (C), two envelope glycoproteins (E1 & E2), a small hydrophobic protein (p7), and six non-structural proteins (NS2, NS3, NS4A, NS4B, NS5A & NS5B). The processing of the polyprotein into the individual proteins is catalyzed by host and viral proteases [Lohmann et al. (1996) J. Hepatol. 24:11-19, Penin et al. (2004) J. Hepatol. 24:11-19].

When a healthy host (human or animal) encounters a foreign antigen (such as proteins derived from a bacterium, virus and/or parasite), the host normally initiates an immune response. The adaptive immune response may be humoral, cellular or both [Whitton at al. (2004) Adv. Virus Res. 63: 181-238]. The cellular response is characterized by the selection and expansion of specific T helper cells and T lymphocytes (CTLs) capable of directly eliminating the cells which contain the antigen. In the case of the humoral response, antibodies are produced by B cells and are secreted into the blood and/or lymph in response to an antigenic stimulus. The antibodies neutralize the antigen, (e.g. a virus) by binding specifically to epitopes on its surface, marking it for destruction by phagocytic cells and/or complement-mediated mechanisms to lyse the infected cells [Carroll (2005) Nature Immunol. 5:981-986]. Helper cells (largely CD4 T cells) provide the helper activity that is required for both CTL (largely CD8 T cells) and 13 cell-mediated antibody responses.

In individuals with chronic viral infections, the immune system does not respond to the incoming pathogen to produce an adaptive immune response to clear the infection and thus the host becomes tolerant to the pathogen. Although the mechanism HCV uses to evade the immune surveillance is not completely understood, several possibilities have been suggested. These include blockage of IRF3-mediated induction of type I IFN by NS3-4A, E2 and NS5A sequences, blocking of PKR (double stranded RNA-activated protein kinase) as well as interference of HCV proteins with the function of NK cells [Rehermann et al. (2005) Nature Rev. Immunol. 5:215-229]. Recent results also show the pivotal role of T cells in the control and eradication of HCV infection [Bowen et al. (2005) Nature 436:946-952; Wieland et al. (2005) J. Virol. 79:9369-9380]. In acute HCV infection, although virus-specific antibodies were detected 7-8 weeks after HCV infection [Pawlotsky (1999) J. Hepatol. 31(suppl):71-79], the role of antibody is not clear, since it has been shown that HCV infection can be resolved in the absence of anti-HCV antibodies in chimpanzees [Cooper et al. (1999) Immunity 10:439-449] and without seroconversion in humans [Post et al. (2004) J. Infect. Dis. 189:1846-1855]. In addition, recent evidence suggests that the failure of individuals to produce detectable levels of CD4+ and CD8+ T-cell responses against HCV resulted in chronic infections [Cooper et al. (1999) supra; Thimme et al. (2001) Proc. Natl. Acad. Sci. USA 99:15661-15668; Thimme et al. (2002) J. Exp. Med. 194:1395-1406; Shoukry et al. (2003) J. Exp. Med. 197: 1645-1655]. An interplay of immune functions such as transcriptional changes in type I IFN-response and immune response against double stranded RNA produced during virus replication have been suggested to occur, but no direct evidence for this effect in the clearance of the virus infection has been observed [Rehermann et al. (2005) supra]. It has been observed that in patients who resolve an HCV infection, the immune system produces strong, multi-epitope-specific CD4+ and CD8+ T cell responses [Rehermann et al. (2005) supra], whereas in patients with chronic HCV infection, the T cell response was late, transient or narrowly focused [Thimme et al. (2001) supra; Diepolder et al. (1995) Lancet 36: 1006-1007; Lechner et al. (2000) J. Exp. Med. 191:1499-1522].

The absence of vigorous T cell responses against HCV antigens, which result in chronic infections may also be due to the lack of proper presentation of the appropriate viral antigen to the host immune system. The success in eliminating the virus may result from the manner in which the antigen is processed and presented by the APCs and the involvement of regulatory T helper cells and cytotoxic T lymphocytes (CTLs).

The major participant in the antigen presenting process is the dendritic cell (DC), which captures and processes the antigens. In addition, DCs express lymphocyte co-stimulatory molecules and migrate to lymphoid organs where they secrete cytokines to initiate immune responses. DCs also control the proliferation of B and T lymphocytes, which are the mediators of immunity [Steinman et al. (1999) Hum. Immunol. 60:562-567]. The generation of a CTL response is critical in the elimination of the virus-infected cells and thus in the resolution of infection.

The encountered antigens are processed differently by the APCs depending on the localization of the antigen [Steinman et al. (1999) supra]. Exogenous antigens are processed within the endosomes of the APC and the generated peptide fragments are presented on the surface of the cell complexed with major histocompatibility complex (MHC) class II molecules. The presentation of this complex to CD4+ T cells results in their activation. As a result, cytokines secreted by helper T cells provide the required soluble factors for activation of B cells to produce antibodies against the exogenous antigen (humoral response).

Conversely, intracellular antigens are processed in the proteasome and the resulting peptide fragments are presented as complexes with MHC class I molecules on the surface of APCs. Following binding of this complex to the T cell receptor (TCR), antigen presentation to CD8+ T cells occurs, which results in a CTL immune response. CTLs can eliminate the virus by killing the infected cells and by the production of factors such as the cytokine interferon-γ (IFN-γ), which acts to inhibit viral replication.

As the virus is actively replicating in individuals with chronic viral infections, viral antigens are produced within host cells and secreted antigens are present in the circulation. In spite of the presence of these antigens there is a lack of an effective immune response against the virus. An effective immune response would involve the production of CTLs, which could recognize a broad array of viral epitopes with high affinity. Thus an appropriate therapeutic vaccine containing viral antigens must be internalized and processed in the appropriate cellular compartment in order for viral peptides to be presented in the groove of MHC class I molecules. The recognition of the viral epitopes in the context of class I presentation would allow the activation, production, and differentiation of CD8+ T cells to functional CTLs that are able to mount an effective response against the viral infection.

Thus a therapeutic vaccine containing viral antigens would be effective if it was processed through the proteasomal pathway and presented via MHC class I [Larsson et al. (2001) Trends Immunol. 22:141-148]. This could be achieved either by producing the antigen within the host cell, or by delivery to the appropriate cellular compartment such that the antigen is processed and presented in a manner that will elicit the desired cellular response. Several approaches have been documented in the literature for the intracellular delivery of antigens, including viral vectors [Lorenz et al. (2001) Hum. Gene Ther. 10:1095-1103], the use of DNA-transfected cells [Donnelly at al. (1997) Annu. Rev. Immunol. 15:617-648] and the expression of the antigen through injected DNA vectors [Lai et al. (1998) Crit. Rev. Immunol. 18:449-484].

By virtue of their APC functionality, DCs which are derived from monocytes, have been shown to have great potential as immune modulators that stimulate primary T cell response [Banchereau et al. (1998) Nature 392:245-252]. This unique property of the DCs to capture, process, and effectively present antigen makes them very important tools for therapeutic vaccine development [Laupeze et al. (1999) Hum Immunol, 60:591-597]. Targeting of the antigen to the DCs is a crucial step and the presence of several receptors on the DCs specific to the Fe region of monoclonal antibodies (mAb) has been exploited for this purpose [Regnault et al. (1999) J. Exp. Med. 189:371-380]. Examples of this approach include ovarian cancer mAb-B43.13 [Berlyn et al. (2001) Clin Immunol. 101: 276-283], anti-PSA mAb, and anti-HBV antibody antigen complexes {Wen et al. (1999) hit. Rev. Immunol. 18:251-258]. Cancer immunotherapy using DCs loaded with tumor-associated antigens have been shown to produce tumor-specific immune responses and anti-tumor activity [Campton et al. (2000) J. Invest. Dermatol. 115:57-61; Fong et al. (2000) Annu. Rev. Immunol. 18:245-273]. Promising results were obtained in clinical trials in vivo using tumor antigen-pulsed DCs [Tarte et al. (1999) Leukemia 13:653-663]. These studies clearly demonstrate the efficacy of using DCs to generate immune responses against cancer antigens. A therapeutic vaccine must be able to elicit host immune responses against viral antigens to which the host immune system is tolerant. This involves the delivery of antigens to DCs, appropriate antigen presentation and priming of HCV-specific CD8+ T cells that can result in therapeutic effect in chronic carriers.

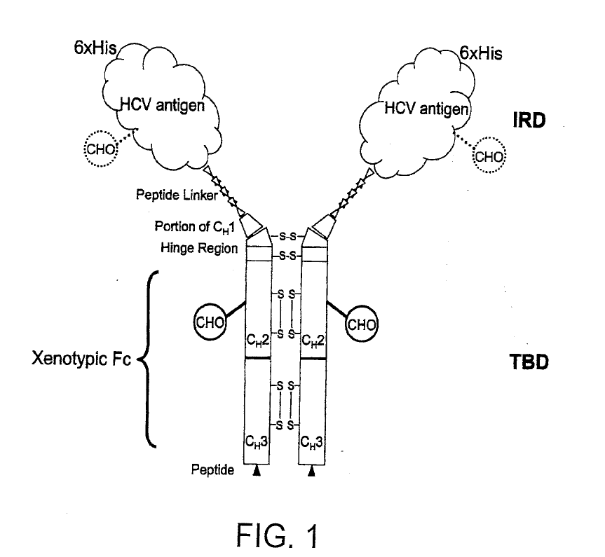

Chimeric antigen vaccines of the invention are a novel class of recombinant “chimeric antigens” produced as fusion proteins of selected antigens and specific regions of an antibody. The bifunctional design of the molecule is tailored to target the viral antigen to APCs, especially DCs, to elicit both humoral and cellular immune responses against the selected antigen. The HCV Chimigen™ vaccine in its dimerized form is schematically represented in FIG. 1.

The vaccine has two domains: an immune response domain (IRD) that contains the recombinant HCV viral antigen, and a target-binding domain (TBD), which contains an Fc fragment of a monoclonal antibody. The design of the vaccine imparts several unique properties to its function. The chimeric design favors the formation of antibody-like structures that facilitate its uptake through specific receptors and results in appropriate antigen presentation. It can be processed through the proteasomal pathway and the peptides presented as complexes with MHC class I, resulting in a CTL response. Chimigen™ vaccines can also be processed via the endosomal pathway, presented by MHC class II, to produce a humoral response.

The TBD mediates the binding of the Chimigen™ vaccine to specific APC receptors such as Fcγ receptors. While the invention is not limited by any particular mechanism of action, it appears that binding of the molecule to Fcγ receptors on APC (e.g., immature DCs) results in the processing of the antigen through the MHC class I pathway. In some embodiments, a xenotypic TBD, the recombinant antigen, the linker peptides of varying lengths incorporated at the amino and carboxy termini of the antigen, make the whole molecule “foreign” and allow the host immune system to mount multi-epitopic immune responses against the fusion protein, including the HCV antigen, Fusion protein Chimigen3 proteins can also be produced in non-mammalian cells (e.g., yeast or insect cells) so that they are glycosylated in an non-mammalian fashion, thereby enhancing their immunogenicity in mammalian (e.g., human) hosts. Mannose/pauci-mannose glycosylation introduced in insect cells also permits the uptake of the vaccine by mannose receptors on APCs for uptake.

Therefore, Chimigen™ vaccines can be internalized by the APCs through specific Fcγ receptors I, II and III (CD64, CD32, CD16), mannose receptors (CD206), other C-type lectin receptors, and by phagocytosis [Geijtenbeek et al. (2004) Annu. Rev. Immunol. 22:33-54]. The uptake via specific receptors, processing through the endosomal and proteasomal pathways, and presentation on both classes of MHC molecules can result in a broad immune response capable of preventing viral infection or eliminating the virus-infected cells. The generation of a CTL response is critical to clear virus-infected cells [Whitton et al. (2004) Adv. Virus Res. 63:181-238]. HepaVaxx B, ViRexx's first Chimigen™ therapeutic vaccine for the treatment of chronic HBV infections, has shown very promising results in preclinical studies [George et al. (2003) A novel class of therapeutic vaccines for the treatment of chronic viral infections: evaluation in ducks chronically infected with duck hepatitis B virus (DHBV), in Hepdart 2003, Frontiers in Drug Development for Viral Hepatitis: December 14-18, Kauai, Hi., USA; George et al. (2003) A novel class of therapeutic vaccines for the treatment of chronic viral infections. International Meeting of the Molecular Biology of Hepatitis B Viruses. September 7-10, Centro Congressi Giovanni XXIII, Bergamo, Italy; George et al. (2004) Immunological Evaluation of a Novel Chimeric Therapeutic Vaccine for the Treatment of Chronic Hepatitis B Infections, (2004) International Meeting of the Molecular Biology of Hepatitis B Viruses. Woods Hole, Mass., USA, Oct. 24-27, 2004; George et al. (2005) BioProcessing Journal 4:39-45; George et al. (2006) A new class of therapeutic vaccines for the treatment of chronic hepatitis B infections. In “Framing the Knowledge of Viral Hepatitis” Schinazi, R. F. Editor, IHL Press USA].

### B. Definitions

The terms used in this application have the meanings indicated by the following definitions (unless otherwise indicated).

“Antibody” refers to an immunoglobulin molecule produced by B lymphoid cells. These molecules are characterized by having the ability to bind specifically with an antigen, each being defined in terms of the other.

“Antibody response” or “humoral response” refers to a type of immune response in which antibodies are produced by B lymphocytes and are secreted into the blood and/or lymph in response to an antigenic stimulus. In a properly functioning immune response, the antibody binds specifically to antigens on the surface of cells (e.g., a pathogen), marking the cell for destruction by phagocytic cells, antibody-dependent cellular cytotoxicity (ADCC) effector cells, and/or complement-mediated mechanisms. Antibodies also circulate systemically and can bind to free virions. This antibody binding can neutralize the virion and prevent it from infecting a cell as well as marking the virion for elimination from host by phagocytosis or filtration in the kidneys.

“Antigen” refers to any substance that, as a result of corning in contact with appropriate cells, induces a state of sensitivity and/or immune responsiveness and that reacts in a demonstrable way with antibodies and/or immune cells of the sensitized subject in vivo or in vitro. Thus, antigens can include, for example, cells or viral particles and/or each of their components. In the case of viruses, the components specifically include viral proteins.

“Antigen-presenting cell” (“APC”) refers to the accessory cells of antigen-inductive events that function primarily by internalizing antigens, processing antigens and presenting antigenic epitopes in context of major histocompatibility complex (MHC) class I or II molecules to lymphocytes. The interaction of APCs with antigens is an essential step in immune induction because it enables lymphocytes to encounter and recognize antigenic molecules and to become activated. Exemplary APCs include macrophages, monocytes, Langerhans cells, interdigitating dendritic cells, Follicular dendritic cells, and B cells.

“B cell” refers to a type of lymphocyte that produces immunoglobulins (antibodies) that interact with antigens.

“C_{H}1 region”, “C_{H}2 region”, “C_{H}3 region” each refer to a different region of the heavy chain constant domain of an antibody.

“Cellular response” or “cellular host response” refers to a type of immune response mediated by specific helper and killer T cells capable of directly or indirectly eliminating virally infected or cancerous cells.

As used herein, the term “chimeric antigen” refers to a polypeptide comprising an immune response domain (IRD) and a target binding domain (TBD). The immune response domain and target binding domains may be directly or indirectly linked by covalent or non-covalent means.

“Complex” or “antigen-antibody complex” refers to the product of the reaction between an antibody and an antigen. Complexes formed with polyvalent antigens tend to be insoluble in aqueous systems.

“Cytotoxic T-lymphocyte” is a specialized type of lymphocyte capable of destroying foreign cells and host cells infected with the infectious agents that produce viral antigens.

“Epitope” refers to the simplest form of an antigenic determinant, on a complex antigen molecule; this is the specific portion of an antigen that is recognized by an antibody or a T cell receptor.

“Fragment” refers to a part of a disunified entity. In the context of this invention it may also be used to refer to that part as part of a corresponding entity. Accordingly, a fusion protein comprising a Fc fragment may refer to a recombinant molecule comprising the same peptide sequence as the native fragment.

“Fusion protein” refers to a protein formed by expression of a hybrid gene made by combining two or more coding sequences.

“Hinge region” refers to the portion of an antibody that connects the Fab fragment to the Fc fragment; the hinge region contains disulfide bonds that covalently link the two heavy chains together to form a dimeric molecule.

The term “homolog” refers to a molecule which exhibits homology to another molecule, by for example, having sequences of chemical residues that are the same or similar at corresponding positions. The phrase “% homologous” or “% homology” refers to the percent of nucleotides or amino acids at the same position of homologous polynucleotides or polypeptides that are identical or similar. For example, if 75 of 80 residues in two proteins are identical, the two proteins are 93.75% homologous. Percent homology can be determined using various software programs known to one of skill in the art.

“Host” refers to a warm-blooded animal to which a chimeric antigen, for example, can be administered.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases that pair through the formation of hydrogen bonds. Hybridization can occur under varying circumstances. The terms “hybridize”, “hybridizing”, “hybridizes” and the like, used in the context of polynucleotides, are meant to refer to conventional hybridization conditions, preferably such as hybridization in 50% formamide/6×SSC/0.1% SDS/100 μg/mL mDNA, in which temperatures for hybridization are above 37° C. and temperatures for washing in 0.1×SSC/0.1% SDS are above 55° C.

“Immunity” or “immune response” refers to the body's response to an antigen. In particular embodiments, it refers to the ability of the body to resist or protect itself against infectious disease.

“Immune Response Domain (IRD)” refers to the variously configured antigenic portion of a chimeric molecule. The IRD comprises one or more antigens or one or more recombinant antigens. Preferred viral antigens include, but are not limited to, HCV Core, HCV E1-E2, HCV E1, HCV E2, HCV P7, HCV NS3-serine protease, HCV NS4A, HCV NS4B, and HCV NS5A.

As used herein, the phrase “immune-treatable condition” refers to a condition or disease that can be prevented, inhibited or relieved by eliciting or modulating an immune response in the subject.

“Lymphocyte” refers to a subset of nucleated cells found, for example, in the blood, which mediate specific immune responses.

“Monoclonal antibody” or “mAb” refers to an antibody produced from a clone or genetically homogenous population of fused hybrid cells, i.e., a hybridoma cell. Hybrid cells are cloned to establish cells lines producing a specific monoclonal antibody that is chemically and immunologically homogenous, i.e., that recognizes only one type of antigen.

As used herein, “operably linked” means incorporated into a genetic construct so that expression control sequences effectively control expression of a coding sequence of interest.

“Peptide linkage” or “peptide bond” refers to the covalent chemical linkage between two or more amino acids. It is a substituted amide linkage between the α-amino group of one amino acid and the α-carboxyl group of another amino acid.

A “pharmaceutical excipient” comprises a material such as an adjuvant, a carrier, a pH-adjusting and buffering agent, a tonicity adjusting agent, a wetting agent, a preservative, and the like.

“Pharmaceutically acceptable” refers to a non-toxic composition that is physiologically compatible with humans or other animals.

The term “polynucleotide” as used herein refers to a polymeric form of nucleotides of any length, either ribonucleotides or deoxyribonucleotides. This term refers only to the primary structure of the molecule. Thus, the term includes double- and single-stranded DNA and RNA. It also includes known types of modifications, for example, labels which are known in the art, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide.

“Polypeptide” and “protein” are used interchangeably and mean any peptide-linked chain of amino acids, regardless of length or post-translational modification.

As used herein, “prophylaxis” means complete prevention of the symptoms of a disease, a delay in onset of the symptoms of a disease, or a lessening in the severity of subsequently developed disease symptoms.

“Prevention” of a disease means that symptoms of the disease are essentially absent.

“Protease cleavage site” refers to a site at which proteolytic enzymes catalyze the hydrolysis (break) of peptide bonds between amino acids in polypeptide chains.

In the present invention, the phrase “stringent hybridization conditions” or “stringent conditions” refers to conditions under which a compound of the invention will hybridize to its target sequence, but to a minimal number of other sequences.

The term “subject” refers to any warm-blooded animal, preferably a human.

“Tag” refers to a marker or marker sequence used to isolate or purify a molecule containing the tag. An exemplary tag includes a 6×His (i.e., a sequence of six histidines) tag.

“T cell” refers to a type of lymphocyte that can mount an antigen-specific response to an antigen and which plays a role in humoral and cellular immune responses.

“Target Binding Domain (TBD)” refers to all or part of an immunoglobulin heavy chain constant region (e.g., C_{H}1 (all or part)-C_{H}2—C_{H}3).

The phrase “therapeutically effective amount” refers to an amount of an agent (e.g., a chimeric antigen or a polynucleotide encoding a chimeric antigen) sufficient to elicit an effective B cell, cytotoxic T lymphocyte (CTL) and/or helper T lymphocyte (Th) response to the antigen and to block or to cure or at least partially arrest or slow symptoms and/or complications of a disease or disorder. A subset of T cells function as T helper cells by secreting cytokines that help activate B cells to secrete antibodies or help another T cell subset to become effector cytotoxic T lymphocytes (CTLs).

The terms “treating” and “treatment” as used herein cover any treatment of a condition treatable by a chimeric antigen in an animal, particularly a human, and include: (i) preventing the condition from occurring in a subject which may be predisposed to the condition but has not yet been diagnosed as having it; (ii) inhibiting the condition, e.g., arresting or slowing its development; or (iii) relieving the condition, e.g., causing regression of the condition or its symptoms.

As used herein, an agent that is “therapeutic” is an agent that causes a complete abolishment of the symptoms of a disease or a decrease in the severity of the symptoms of the disease.

“Xenotypic” means originating from a species other than the host. For example, a recombinantly expressed antibody cloned from a mouse genome would be xenotypic to a human but not to a mouse, regardless of whether that recombinantly expressed antibody was produced in a bacterial, insect, human, or mouse cell. Thus, in the context of a chimeric antigen of the invention, a xenotypic TBD (e.g., a xenotypic antibody molecule or a xenotypic antibody fragment) is a TBD derived from a species other than the one to which the chimeric antigen.

### C. Chimeric Antigens

A composition of the present invention includes a chimeric antigen comprising an immune response domain (IRD) and a target binding domain (TBD). In preferred embodiments of the invention, the IRD portion is capable of inducing humoral and/or T cell responses, and the target binding portion is capable of binding an APC, such as a dendritic cell. The chimeric antigen of the present invention may also include one or more of the following: a hinge region of an immunoglobulin (or a segment thereof), a C_{H}1 region of an immunoglobulin (or a segment thereof), a peptide linker, a protease cleavage site, and a tag suitable for use with a purification protocol. A chimeric antigen of the present invention is capable of binding to and activating an APC. Generally, but not necessarily, the IRD is N-terminal of the TBD.

In some embodiments of the invention, the IRD of the chimeric antigen includes one or more (e.g., 2, 3, 4, 5, 6, 7, 8, 9, or 10) proteins (antigens) selected from the group comprising: one or more HCV proteins such as those described herein or one or more recombinant HCV proteins. Between such proteins there can optionally be a linker such as any of the linkers disclosed herein. In the chimeric antigen of the invention, immunogenic fragments of these antigens, rather than the full-length antigens, can be uses. Where more than one antigen is present in a chimeric antigen, only full-length, only immunogenic fragments, or mixtures of full-length antigens and full-length proteins can be used.

The chimeric antigens of the invention can be monomeric (i.e., they contain a single unit comprising an IRD and a TBD) or they can be multimeric (i.e., they can contain multiple units, each comprising an IRD and a TED). Multimers can be, for example, dimers, trimers, tetramers, pentamers, hexamers, septamers, or octamers. In such multimers, the individual units can be identical or different or some can be identical and others different. FIG. 1 depicts a dimeric chimeric antigen.

In yet another embodiment of the invention, the IRD of the chimeric antigen includes a 6×His-peptide fused to one or more HCV proteins, or one or more recombinant HCV proteins.

In some embodiments of the invention, the TBD of the chimeric antigen can be an antibody fragment. The TBD can be of the same species as the host (subject) to which the relevant chimeric antigen is to be administered. On the other hand, in preferred embodiments of the invention, the TED of the chimeric antigen is an antibody fragment xenotypic to the host. For example, if the host is a human, an exemplary xenotypic antibody fragment is a non-human animal antibody fragment, such as a mouse antibody fragment. In certain embodiments of the invention, the xenotypic antibody fragment comprises a murine Fc fragment. In the most preferred embodiments of the invention, the TBD comprises a xenotypic Fc fragment (or a segment thereof), a hinge region (or a segment thereof), a C_{H}1 region (or a segment thereof), and a peptide linkage suitable for linking the target binding domain to the IRE.

The present invention also comprises the use of linking molecules to join the IRD to the TED. Exemplary linker molecules include leucine zippers, and biotin/avidin. Other linkers that can be used (for example in fusion proteins) are peptide sequences. Such peptide linkers, are generally about two to about 40 amino acids (e.g., about 4-10 amino acids) in length. An exemplary peptide linkers include the amino acid sequence SRPQGGGS (SEQ ID NO:1). Other linkers are well known in the art and are generally so glycine and/or alanine rich to allow for flexibility between the regions they join. Generally, in the chimeric antigens of the invention, the IRD and the TBD are not joined by a physical antigen-antibody interaction between an antigen binding part of the TBD (e.g., an antibody molecule or fragment of an antibody molecule) and an appropriate antigenic epitope on the IRD.

In one embodiment, the chimeric antigen of the present invention is a fusion protein having two portions, namely an IRD containing an antigenic sequence (such as a viral antigen(s)), and a TBD containing a xenotypic Fc fragment. The xenotypic murine Fc fragment binds to specific receptors on APC, specifically dendritic cells. The binding region of the chimeric antigen thus targets antigen-presenting cells specifically. The internal machinery of the APC then processes the chimeric antigen and presents specific peptides on MHC class I and class II molecules to contact and activate T cells and generate humoral and cellular immune responses to clear infected cells or other appropriate undesirable cells, e.g., cancer cells.

In a further embodiment, the chimeric antigen can be a fusion protein having two portions, namely a modified viral antigen or antigens, antigenic protein fragments or peptides, or any of these with glycosylation at specific sites, and a xenotypic murine Fc fragment, which can also be glycosylated.

In yet another embodiment, the invention provides a further modified chimeric antigen, wherein the antigen (IRD) is biotinylated and the TBD (e.g., Fc fragment) is conjugated with avidin (e.g., streptavidin) in, for example, a fusion protein. Such an avidin-conjugated TBD facilitates the production of a wide assortment of IRD-TBD conjugates. Naturally it is appreciated that the IRD can be conjugated with avidin (e.g., in the form of a fusion protein) and the TBD (e.g., Fc fragment) can be biotinylated.

In yet another embodiment, the invention provides an association between the IRD (antigen) and the TBD (e.g., antibody Fc fragment) through chemical conjugation.

An embodiment of the present invention includes the use of recombinant antigens of HCV fused to an antibody fragment by molecular biological techniques, production of the fusion proteins in a baculovirus expression system and their use as therapeutic vaccines against chronic HCV infections. The present invention provides an efficient method to deliver a HCV antigen to APCs in vivo so as to generate a broad immune response, a Th1 response involving CTLs and a Th2 (antibody) response. The immunogenicity of pre-selected viral antigen (e.g., one unrecognized by a host immune system) can be increased by the presence of a xenotypic antibody fragment as well as by the presence of specific glycosylation introduced in the insect cell expression system. The antigen-antibody fragment fusion protein, due to the presence of the antibody component, will bind to specific receptors present on various cells of the immune system (e.g., APC), including dendritic cells, macrophages, monocytes, B cells, and granulocytes. The fusion proteins administered to either humans or animals will be internalized by APCs, especially DCs, will be hydrolyzed to small peptides and presented on the cell surface, complexed with MHC Class I and/or MHC Class II molecules to T cells have antigen specific T cell receptors (TCR) of the appropriate specificity. In this way the chimeric antigens (fusion proteins) can elicit a broad immune response and clear the viral infection.

As used herein, the term “Target Binding Domain (TBD)” refers to all or part of an immunoglobulin heavy chain constant region, which is an antibody fragment capable of binding to an Fc receptor on an APC. In accordance with the present invention, the TBD is a protein capable of binding to an Fc receptor on an APC, particularly a dendritic cell, and is subsequently transported into the APC by receptor-mediated uptake. In accordance with the present invention, the presence of an Fc fragment augments the uptake of the chimeric antigen through the Fc receptor on APCs, specifically DC. By virtue of the specific uptake, the viral antigen is processed and presented as foreign; thus, an immune response is effectively elicited to the viral antigen that, on its own, was tolerated by the host or elicited a very weak immune response in the host.

Also, in accordance with the present invention, the chimeric antigen, preferably, is capable of binding to a macrophage mannose receptor/C-type lectin receptors. The macrophage mannose receptor (MMR), also known as CD206, is expressed on APC such as DCs. This molecule is a member of the C-type lectin family of endocytic receptors. Mannosylated chimeric antigen can be bound and internalized by CD206. In general, exogenous antigen is thought to be processed and presented primarily through the MHC class II pathway. However, in the case of targeting through CD206, there is evidence that both the MHC class I and class II pathways are involved [Apostolopoulos et al. (2000) Bur. J. Immunol. 30:1714; Apostolopoulos et al. (2001) Curr. Mol. Med. 1:469; Ramakrishna et al. (2004) J. Immunol. 172:2845-28521. Thus, monocyte-derived dendritic cells loaded with chimeric antigen that specifically targets CD206 will induce both a potent class I-dependent CD8⁺ CTL response and a class II-dependent proliferative T helper response [Ramakrishna et al. (2004) J. Immunol. 172(5):2845-52].

An exemplary TBD is derived from Mouse anti-HBVsAg mAb (Hybridoma 2C12) as cloned in pFastBac HTa expression vector, and expressed in an insect cell expression system (Invitrogen, Carlsbad, Calif., USA). This TBD consists of part of C_{H}1 (having the amino acid sequence VDKKI; SEQ ID NO:2), and Hinge-C_{H}2-C_{H}3 from N-terminal to C-terminal of the mouse anti-HBV sAg mAb. The constant region of the IgG1 molecule for the practice of the present invention can contain a linker peptide, part of C_{H}1-hinge and the regions C_{H}2 and C_{H}3. The hinge region portion of the monomeric TBD can form disulphide bonds with a second TBD molecule. The protein can be expressed as an N-terminal fusion protein with a 6×His tag, a seven amino acid rTEV (recombinant tobacco etch virus) protease cleavage site and the N-terminal fusion of the Target Binding Domain (TBD) of the xenotypic (murine) mAb raised against HBV sAg (Hybridoma 2C12). The exemplary TBD is a fragment of the constant chain of the IgG1 mAb from 2C12 with the sequence of amino acids comprising the 8 amino acid peptide linker, five amino acids of the C_{H}1 region, the hinge sequences, C_{H}2 and C_{H}3 region sequences and, optionally, a C-terminal peptide of ten additional amino acids encoded by nucleotides derived from the expression vector. The exemplary TBD fragment defined herein forms the parent molecule for the generation of fusion proteins with antigens derived from HCV virus.

D. Novel Polynucleotides

Another aspect of the invention provides polynucleotides encoding all of the chimeric antigens disclosed herein. The polynucleotides comprise a first polynucleotide portion encoding an immune response domain and a second polynucleotide portion encoding a target binding domain. The first and second polynucleotide portions may be located on the same or different nucleotide chains.

In addition to the above described regions of the chimeric antigens of the invention, polynucleotides of the invention generally contain leader sequences encoding leader peptides that facilitate secretion of the chimeric antigen from a cell (e.g., a yeast or insect cell) producing it. The relevant leader sequence is generally cleaved from the chimeric antigen prior to secretion from the cell. Leader sequences can be any of those disclosed herein and others known in the art, for example, AcNPV chitinase signal sequence having the amino acid sequence MPLYKLLNVLWLVAVSNAI (SEQ ID NO:37) encoded by the nucleotide sequence ATGCCCTTGTACAAATTGTTAAACGTTTTGTGGTTGGTCGCCGTTTCTAACGC GATT (SEQ ID NO:38) useful for expression in insect cells and the alpha-mating factor leader useful for expression in yeast cells (e.g., Pichia pastoris yeast cells).

The invention provides polynucleotides corresponding or complementary to genes encoding chimeric antigens, mRNAs, and/or coding sequences, preferably in isolated form, including polynucleotides encoding chimeric antigen variant proteins; DNA, RNA, DNA/RNA hybrids, and related molecules, polynucleotides or oligonucleotides complementary or having at least a 90% homology to the genes encoding a chimeric antigen or mRNA sequences or parts thereof; and polynucleotides or oligonucleotides that hybridize to the genes encoding a chimeric antigen, mRNAs, or to chimeric antigen-encoding polynucleotides.

Additionally, the invention includes analogs of the genes encoding a chimeric antigen specifically disclosed herein. Analogs include, e.g., mutants, that retain the ability to elicit an immune response, and preferably have homology of at least 80%, more preferably 90%, and most preferably 95% to any of polynucleotides encoding a chimeric antigen, as specifically described by the sequences set forth in SEQ ID NOs: 39 and 41-51. Typically, such analogs differ by only 1 to 10 codon changes. Examples include polypeptides with minor amino acid variations from the natural amino acid sequence of a viral antigen or of an antibody fragment; in particular, conservative amino acid replacements. Conservative replacements are those that take place within a family of amino acids that are related in their side chains. Genetically-encoded amino acids are generally divided into four families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cystine, serine, threonine, tyrosine. Phenylalanine, tryptophan, and tyrosine are sometimes classified jointly as aromatic amino acids. For example, it is reasonable to expect that an isolated replacement of a leucine with an isoleucine or valine, an aspartate with a glutamate, a threonine with a serine, or a similar conservative replacement of an amino acid with a structurally related amino acid will not have a major effect on biological activity. Polypeptide molecules having substantially the same amino acid sequence as any of the polypeptides disclosed herein but possessing minor amino acid substitutions that do not substantially affect the ability of the chimeric antigens to elicit an immune response, are within the definition of a chimeric antigen. Derivatives include aggregative conjugates with other chimeric antigen molecules and covalent conjugates with unrelated chemical moieties. Covalent derivatives are prepared by linkage of functionalities to groups that are found in chimeric antigen amino acid chains or at the N- or C-terminal residues by means known in the art.

Amino acid abbreviations are provided in Table 1.

TABLE 1
Amino Acid Abbreviations

| | | |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Conservative amino acid substitutions can be made in a protein without altering either the conformation or the function of the protein. Proteins of the invention, or useful for the invention, can comprise not more than 15 (e.g., not more than: 14; 13; 12; 11; 10; 9; 8; 7; 6; 5; 4; 3; 2; or 1) conservative substitution(s). Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant Still other changes can be considered “conservative” in particular environments [see, e.g. Biochemistry 4^{th} Ed., Lubert Stryer ed. (W. H. Freeman and Co.), pages 18-23; Henikoff et al. (1992) Proc Natl Acad Sci USA 89:10915-10919; Lei et al. (1995) J. Biol. Chem. 270:11882-11885].

Additional analog polynucleotides include those with one or more (e.g., 2, 3, 4, 5, 6, 7, 8, 9, 10, 12, 15, or 20) additions or deletions in any of the TBDs and/or any of the IRDs that serve, for example, to increase the solubility of the relevant chimeric antigen. The additions or deletions can be of one or more (e.g., 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, or more) amino acids in the chimeric antigens encoded by the polynucleotides (and the corresponding numbers of nucleotides in the polynucleotides themselves).

The invention also includes polynucleotides that selectively hybridize to polynucleotides that encode chimeric antigens. Preferably a polynucleotide of the invention will hybridize under stringent conditions to one or more of the sequences set forth in SEQ ID NOs:39 and 41-51. Stringency of hybridization reactions is readily determinable by one of ordinary skill in the art and generally is an empirical calculation dependent upon probe length, washing temperature, and salt concentration. In general longer probes require higher temperatures for proper annealing, while shorter probes need lower temperatures. Hybridization generally depends on the ability of denatured nucleic acid sequences to re-anneal when complementary strands are present in an environment below their melting temperature. The higher the degree of desired homology between the probe and hybridizable sequence, the higher the relative temperature that can be used. As a result, it follows that higher relative temperatures would tend to make the hybridization conditions more stringent, while lower temperatures less so. For additional details and explanation of stringency of hybridization reactions, see, e.g., Ausubel et al., Current Protocols in Molecular Biology, Wiley Interscience Publishers, (01995, as Supplemented April 2004, Supplement 66) at pages 2.9.1-2.10.8 and 4.9.1-4.9.13.

“Stringent conditions” or “high stringency conditions”, as defined herein, are identified by, but not limited to, those that (1) employ low ionic strength and high temperature for washing, for example 0.015 M sodium chloride/0.0015 M sodium citrate/0.1% sodium dodecyl sulfate at 50° C.; (2) employ, during hybridization, a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. “Moderately stringent conditions” are described by, but not limited to, those in Sambrook et al., Molecular Cloning: A Laboratory Manual, 2^{nd} Ed., New York: Cold Spring Harbor Press, 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

Embodiments of a polynucleotide of the invention include: a polynucleotide encoding a chimeric antigen having a sequence selected from any of the sequences as set forth in SEQ ID NOs: 40 and 52-62, a nucleotide sequence of chimeric antigen selected from any of the sequences as set forth in SEQ ID NOs: 39 and 41-51 but with T nucleotides substituted with U nucleotides. For example, embodiments of chimeric antigen nucleotides comprise, without limitation:
(a) a polynucleotide comprising or consisting of a sequence selected from any of the sequences as set forth in SEQ ID NOs: 39 and 41-51, wherein T can also be U;
(b) a polynucleotide whose sequence is at least 80% homologous to a sequence selected from any of the sequences as set forth in SEQ ID NOs: 39 and 41-51;
(c) a polynucleotide that encodes a chimeric antigen whose sequence is encoded by a DNA contained in any of the plasmids disclosed herein;
(d) a polynucleotide that encodes a chimeric antigen whose sequence is a sequence selected from any of the sequences as set forth in SEQ ID NOs: 40 and 52-62;
(e) a polynucleotide that encodes a chimeric antigen-related protein that is at least 90% identical to an entire amino acid sequence whose sequence is selected from any of the sequences as set forth in SEQ ID NOs: 40 and 52-62;
(f) a polynucleotide that is fully complementary to a polynucleotide of any one of (a)-(e);
(g) a polynucleotide that selectively hybridizes under stringent conditions to a polynucleotide of (a)-(f); and
(h) a polynucleotide comprising or consisting of a sequence selected from any of the sequences as set forth in SEQ ID NOs: 39 and 41-51 but lacking all or some of the sequences other than the IRD (e.g., the HCV proteins listed herein) and the TBD and, optionally containing, for example, one or more alternative linkers and/or an alternative secretory (leader) peptide. In addition, additional sequences (e.g., vector-derived sequences encoding amino acids at the C terminus of the TBD) can be deleted from polynucleotides of the invention. Such additional sequences can be those encoding 1-15 (e.g., 2, 3, 4, 5, 6, 7, 8, 9, 10, 11, 12, 13, or 14) amino acids.

The invention also provides recombinant DNA or transcribed RNA molecules containing a chimeric antigen polynucleotide, an analog or homologue thereof, including but not limited to phages, plasmids, phagemids, cosmids, YACs (yeast artificial chromosomes), BACs (bacterial artificial chromosomes), as well as various viral and non-viral vectors well known in the art, and cells transformed or transfected with such recombinant DNA or RNA molecules. Methods for generating such molecules are well known [see, for example, Sambrook et al., 1989, supra].

The invention further provides a host-vector system comprising a recombinant DNA molecule containing a chimeric antigen polynucleotide, analog or homologue thereof within a suitable prokaryotic or eukaryotic host cell. Examples of suitable eukaryotic host cells include a yeast cell, a plant cell, or an animal cell, such as a mammalian cell or an insect cell (e.g., a baculovirus-infectible cell such as an Sf9, Sf21, ExpresSF⁺®, Drosophila S2 or High Five™ cell). Examples of suitable mammalian cells include various prostate cancer cell lines such as DU145 and TsuPr1, other transfectable or transducible prostate cancer cell lines, primary cells (PrEC), as well as a number of mammalian cells routinely used for the expression of recombinant proteins (e.g., COS, CHO, 293, 293T cells). More particularly, a polynucleotide comprising the coding sequence of chimeric antigen or a fragment, analog or homolog thereof can be used to generate chimeric antigen thereof using any number of host-vector systems routinely used and widely known in the art.

A wide range of host-vector systems suitable for the expression of chimeric antigens thereof are available, see for example, Sambrook et al., 1989, supra; Ausubel, Current Protocols in Molecular Biology, 1995, supra). Preferred vectors for insect cell expression include, but are not limited to, the transfer vector plasmid pFastBac HTa (Invitrogen). Using such transfer vector plasmids, recombinant baculoviruses can be produced in insect cells and these can be used to infect several insect cell lines, including for example Sf9, Sf21, ExpresSF⁺®, Drosophila S2 or High Five™, to express chimeric antigens. An example of this is the Bac to Bac baculovirus expression system (Invitrogen). Alternatively, preferred yeast expression systems include Saccharomyces cerevisiae, Schizosaccharomyces pombe, Pichia pastoris, and Pichia august. The host-vector systems of the invention are useful for the production of a chimeric antigen.

A chimeric antigen or an analog or homolog thereof can also be produced by the stable transfection of cells (e.g., insect cells) with a plasmid construct containing the an appropriate promoter (e.g., an insect cell promoter) and encoding a chimeric antigen. For example, a recombinant plasmid pMIB-V5 (Invitrogen) encoding chimeric antigen or an analog or homolog thereof can be used for stable transfection of SD insect cells. The chimeric antigen or related protein is expressed in the Sf9 cells, and the chimeric antigen is isolated using standard purification methods. Various other expression systems well known in the art can also be employed. Expression constructs encoding a leader peptide joined in frame to the chimeric antigen coding sequence can be used for the generation of a secreted form of chimeric antigen.

As discussed herein, redundancy in the genetic code permits variation in chimeric antigen gene sequences. In particular, it is known in the art that specific host species often have specific codon preferences, and thus one can adapt the disclosed sequence as preferred for a desired host. For example, preferred analog codon sequences typically have rare codons (i.e., codons having a usage frequency of less than about 20% in known sequences of the desired host) replaced with higher frequency codons. Codon preferences for a specific species are calculated, for example, by utilizing codon usage tables available on the INTERNET such as at world wide web URL www.kazusa.or.jp/codon.

Additional sequence modifications are known to enhance protein expression in a cellular host. These include elimination of sequences encoding spurious polyadenylation signals, exon/intron splice site signals, transposon-like repeats, and/or other such well-characterized sequences that are deleterious to gene expression. The GC content of the sequence is adjusted to levels average for a given cellular host, as calculated by reference to known genes expressed in the host cell. Where possible, the sequence is modified to avoid predicted hairpin secondary mRNA structures. Other useful modifications include the addition of a translational initiation consensus sequence at the start of the open reading frame, as described in Kozak [(1989) Mol. Cell. Biol. 9:5073-5080]. Skilled artisans understand that the general rule that eukaryotic ribosomes initiate translation exclusively at the 5′ proximal AUG codon is abrogated only under rare conditions [see, e.g., Kozak (1995) Proc. Natl. Acad. Sci. USA 92:2662-2666; Kozak (1987) Nucl. Acids Res. 15:8125-8148].

Escherichia coli clones, each transformed with one of the plasmids listed below, were deposited on Oct. 11, 2006, under the Budapest Treaty at the International Depository Authority of Canada (IDAC), 1015 Arlington Street Winnipeg, Manitoba, R3E 3R2 Canada (telephone no.: (204) 789-6030; facsimile no.: (204) 789-2018). Each clone is readily identified by the indicated IDAC accession number.

| Plasmid | IDAC accession number |
|---|---|
| pFastBacHTa-gp64 HCV NS3mutS-TBD | 111006-01 |
| pFastBacHTa-gp64 HCV NS3mut-TBD | 111006-02 |
| pFastBacHTa-gp64 NS3-NS5A-TBD | 111006-03 |
| pFastBacHTa-gp64 HCV NS5A-TBD | 111006-04 |
| pFastBacHTa HCV NS3mut-TBD | 111006-05 |
| pFastBacHTa HCV NS3-NS4B-NS5A-TBD | 111006-06 |

The samples deposited with the IDAC are taken from the same deposit maintained by the ViRexx Medical Corporation since prior to the filing date of this application. The deposits will be maintained without restriction in the IDAC depository for a period of 30 years, or 5 years after the most recent request, or for the effective life of the patent, whichever is longer, and will be replaced if the deposit becomes non-viable during that period.

E. Pharmaceutical Compositions of the Invention

One aspect of the invention relates to pharmaceutical compositions comprising a pharmaceutically acceptable excipient and a chimeric antigen comprising an immune response domain and a target binding domain, wherein the target binding domain comprises an antibody fragment. In therapeutic applications, the pharmaceutical compositions can be administered to a subject in an amount sufficient to elicit an effective B cell, cytotoxic T lymphocyte (CTL) and/or helper T lymphocyte (Th) response to the antigen and to prevent infection or to cure or at least partially arrest or slow symptoms and/or complications of infection. Amounts effective for this use will depend on, e.g., the particular composition administered, the manner of administration, the stage and severity of the disease being treated, the weight and general state of health of the subject, and the judgment of the prescribing physician.

The dosage for an initial therapeutic immunization (with chimeric antigen) generally occurs in a unit dosage range where the lower value is about 1, 5, 50, 500, or 1,000 ng and the higher value is about 10,000; 20,000; 30,000; or 50,000 μg. Dosage values for a human typically range from about 500 ng to about 50,000 μg per 70 kilogram subject. Boosting dosages of between about 1.0 ng to about 50,000 μg of chimeric antigen pursuant to a boosting regimen over days to months may be administered depending upon the subject's response and condition. Administration should continue until at least clinical symptoms or laboratory tests indicate that the condition has been prevented, arrested, slowed or eliminated and for a period thereafter. The dosages, routes of administration, and dose schedules are adjusted in accordance with methodologies known in the art.

A human unit dose form of a chimeric antigen is typically included in a pharmaceutical composition that comprises a human unit dose of an acceptable carrier, in one embodiment an aqueous carrier, and is administered in a volume/quantity that is known by those of skill in the art to be useful for administration of such polypeptides to humans (see, e.g., Remington: The Science and Practice of Pharmacy, 20^{th} Edition, A. Gennaro, Editor, Lippincott Williams & Wilkins, Baltimore, Md., 2000). As appreciated by those of skill in the art, various factors can influence the ideal dose in a particular case. Such factors include, for example, half life of the chimeric antigen, the binding affinity of the chimeric antigen, the immunogenicity of the composition, the desired steady-state concentration level, route of administration, frequency of treatment, and the influence of other agents used in combination with the treatment method of the invention, as well as the health status of a particular subject.

Generally, sufficient chimeric antigen to elicit an immune response to the chimeric antigen is administered to a subject. The TBD targets the chimeric antigen to specific receptors on APCs, such as DCs. The chimeric antigen is internalized, processed through antigen presentation pathways to elicit both humoral as well as cellular immune responses.

In certain embodiments, the compositions of the present invention are employed in serious disease states, that is, life-threatening or potentially life-threatening situations. In such cases, as a result of the relative nontoxic nature of the chimeric antigen in preferred compositions of the invention, it is possible and may be felt desirable by the treating physician to administer substantial excesses of these chimeric antigens relative to these stated dosage amounts.

The concentration of chimeric antigen of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The pharmaceutical compositions can be delivered via any route known in the art, such as parenterally, intrathecally, intravascularly, intravenously, intramuscularly, transdermally, intradermally, subcutaneously, intranasally, topically, orally, rectally, vaginally, pulmonarily or intraperitoneally. Preferably, the composition is delivered by parenteral routes, such as subcutaneous or intradermal administration.

The pharmaceutical compositions can be prepared by mixing the desired chimeric antigens with an appropriate vehicle suitable for the intended route of administration. In making the pharmaceutical compositions of this invention, the chimeric antigen is usually mixed with an excipient, diluted by an excipient or enclosed within a carrier that can be in the form of a capsule, sachet, paper or other container. When the pharmaceutically acceptable excipient serves as a diluent, it can be a solid, semi-solid, or liquid material, which acts as a vehicle, carrier or medium for the therapeutic agent. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the chimeric antigen, soft and hard gelatin capsules, suppositories, sterile injectable solutions, and sterile packaged powders.

Some examples of suitable excipients include, but are not limited to, dextrose, sucrose, glycerol, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the chimeric antigen after administration to the subject by employing procedures known in the art. See, e.g., Remington, supra, at pages 903-92 and pages 1015-1050.

For preparing solid compositions such as tablets, the chimeric antigen is mixed with a pharmaceutical excipient to form a solid preformulation composition containing a homogeneous mixture of a chimeric antigen of the present invention. When referring to these preformulation compositions as homogeneous, it is meant that the chimeric antigen is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

In preparing a composition for parenteral administration strict attention must be paid to tonicity adjustment to reduce irritation. A reconstitutable composition is a sterile solid packaged in a dry form. A reconstitutable composition is preferred because it is more stable when stored as a dry solid rather than in a solution ready for immediate administration. The dry solid is usually packaged in a sterile container with a butyl rubber closure to ensure the solid is kept at an optimal moisture range. A reconstitutable dry solid is formed by dry fill, spray drying, or freeze-drying methods. Descriptions of these methods may be found, e.g., in Remington, supra, at pages 681-685 and 802-803.

Compositions for parenteral injection are generally dilute, and the component present in the higher proportion is the vehicle. The vehicle normally has no therapeutic activity and is nontoxic, but presents the chimeric antigen to the body tissues in a form appropriate for absorption. Absorption normally will occur most rapidly and completely when the chimeric antigen is presented as an aqueous solution. However, modification of the vehicle with water-miscible liquids or substitution with water-immiscible liquids can affect the rate of absorption. Preferably, the vehicle of greatest value for this composition is isotonic saline. In preparing the compositions that are suitable for injection, one can use aqueous vehicles, water-miscible vehicles, and nonaqueous vehicles

Additional substances may be included in the injectable compositions of this invention to improve or safeguard the quality of the composition. Thus, an added substance may affect solubility, provide for subject comfort, enhance the chemical stability, or protect the preparation against the growth of microorganisms. Thus, the composition may include an appropriate solubilizer, substances to act as antioxidants, and substances that act as a preservative to prevent the growth of microorganisms. These substances will be present in an amount that is appropriate for their function, but will not adversely affect the action of the composition. Examples of appropriate antimicrobial agents include thimerosal, benzethonium chloride, benzalkonium chloride, phenol, methyl p-hydroxybenzoate, and propyl p-hyrodxybenzoate. Appropriate antioxidants may be found in Remington, supra, at p. 1015-1017.

In certain embodiments, liposomes, nanocapsules, microparticles, lipid particles, vesicles, and the like, are used for the administration of the chimeric antigens of the present invention. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

Compositions administered via liposomes may also serve: 1) to target the chimeric antigen to a particular tissue, such as lymphoid tissue; 2) to target selectively to APCs; 3) to carrier additional stimulatory or regulatory molecules; or 4) to increase the half-life of the chimeric antigen composition. Liposomes include emulsions, foams, micelles, insoluble monolayers, liquid crystals, phospholipid dispersions, lamellar layers and the like. In these preparations, the chimeric antigen to be delivered is incorporated as part of a liposome, alone or in conjunction with a molecule that binds to a receptor prevalent among lymphoid cells, such as monoclonal antibodies that bind to the CD45 antigen, or with other therapeutic or immunogenic compositions. Thus, liposomes either filled or decorated with a desired chimeric antigen of the invention can be directed to the site of lymphoid cells, where the liposomes then deliver the chimeric antigens.

Liposomes for use in accordance with the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes for the desired route of administration, e.g., in the blood stream. A variety of methods are available for preparing liposomes [as described in, e.g., Szoka et al. (1980) Ann. Rev. Biophys. Bioeng. 9:467-508; and U.S. Pat. Nos. 4,235,871, 4,501,728, 4,837,028, and 5,019,369]. A liposome suspension containing a chimeric antigen may be administered intravenously, locally, topically, etc. in a dose which varies according to, inter alia, the manner of administration, the chimeric antigen being delivered, and the stage of the disease being treated.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described herein. The compositions can be administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

Another formulation employed in the methods of the present invention employs transdermal delivery devices (“patches”). Such transdermal patches may be used to provide continuous or discontinuous infusion of the chimeric antigen of the present invention in controlled amounts. The construction and use of transdermal patches for the delivery of pharmaceutical agents is well known in the art. See, for example, U.S. Pat. No. 5,023,252, herein incorporated by reference. Such patches may be constructed for continuous, pulsatile, or on demand delivery of pharmaceutical agents.

Additionally, it may be advantageous to include at least one antiviral therapeutic or chemotherapeutic in addition to the chimeric antigen and pharmaceutical excipient. These include, but are not limited to, interferon-α 2a/b, and antiviral agents such as ribavirin.

In some embodiments it may be desirable to include in the pharmaceutical compositions of the invention at least one component which primes B-lymphocytes or T lymphocytes. Lipids have been identified as agents capable of priming CTL in vivo. For example, palmitic acid residues can be attached to the ε- and α-amino groups of a lysine residue and then linked, e.g., via one or more linking residues such as Gly, Gly-Gly-, Ser, Ser-Ser, or the like, to an immunogenic peptide. The lipidated peptide can then be administered either directly in a micelle or particle, incorporated into a liposome, or emulsified in an adjuvant, e.g., incomplete Freund's adjuvant. In a preferred embodiment, a particularly effective immunogenic composition comprises palmitic acid attached to e- and α-amino groups of Lys, which is attached via linkage, e.g., Ser-Ser, to the amino terminus of the immunogenic peptide.

As another example of lipid priming of CTL responses, E. coli lipoproteins, such as tripalmitoyl-S-glycerylcysteinlyseryl-serine (P₃CSS) can be used to prime virus specific CTL when covalently attached to an appropriate peptide [see, e.g., Deres et al. (1989) Nature 342:561]. Chimeric antigens of the invention can be coupled to P₃CSS, for example, and the lipopeptide administered to an individual to specifically prime an immune response to the target antigen.

While the compositions of the present invention should not require the use of adjuvants, adjuvant can be used. Various adjuvants may be used to increase the immunological response, depending on the host species, and including but not limited to Freund's (complete and incomplete), mineral gels such as aluminum hydroxide, surface active substances such as lysolecithin, detergents, pluronic polyols, polyanions, peptides, oil emulsions, keyhole limpet hemocyanins, dinitrophenol, immunostimulatory polynucleotide sequences, and potentially useful human adjuvants such as BCG (bacille Calmette-Guerin) and corynebacterium parvum. Additional adjuvants are also well known in the art.

F. Methods of Using Chimeric Antigens

Another aspect of the invention provides methods of enhancing antigen presentation by APCs, said method comprising administering, to the APCs, a chimeric antigen that comprises an immune response domain and a target binding domain, wherein the target binding domain comprises an antibody fragment (e.g., a xenotypic antibody fragment). In a preferred embodiment, the APCs are dendritic cells.

An aspect of the invention relates to methods of activating APCs comprising contacting the APC with a chimeric antigen that comprises an immune response domain and a target binding domain, wherein the target binding domain comprises an antibody fragment (e.g., a xenotypic antibody fragment). In a preferred embodiment, the APC is contacted with the chimeric antigen in vivo. In another preferred embodiment, the contacting takes place in a human.

Yet another aspect of the invention provides methods of eliciting an immune response, said method comprising administering to an animal a chimeric antigen that comprises an immune response domain and a target binding domain, wherein the target binding domain comprises an antibody fragment (e.g., a xenotypic antibody fragment). The immune response can be a humoral and/or cellular immune response. In a preferred embodiment, the cellular immune response is a Th 1, a Th2, and/or a CTL response.

Another aspect of the invention provides methods of treating immune-treatable conditions comprising administering, to an animal in need thereof, a chimeric antigen that comprises an immune response domain and a target binding domain, wherein the target binding domain comprises a xenotypic antibody fragment. Preferably, the immune-treatable condition is a chronic hepatitis C viral infection. For the treatment of HCV, preferably the immune response domain comprises a protein selected from the group consisting of a HCV Core (1-191) protein, a HCV Core (1-177) protein, a HCV E1 protein, a HCV E2 protein, a HCV E1-E2 protein, a HCV NS2 protein, a HCV NS3 protein, a HCV NS4A protein, a HCV NS4B protein, a HCV NS5A protein, a HCV NS5B protein, a HCV p7 protein, and combinations thereof.

Another aspect of the invention provides methods of vaccinating an animal against a viral infection comprising administering to the animal a chimeric antigen that comprises an immune response domain and a target binding domain, wherein the target binding domain comprises an antibody fragment. The method of the invention can prophylactically or therapeutically vaccinate the animal against the viral infection.

The present invention also comprises methods of using the compositions of the present invention to bind and activate APCs, such as DCs. The present invention also comprises methods of using the compositions of the present invention to activate T cells. The present invention also comprises a method of delivering an antigen to an immune system cell, such as an APC. The present invention also comprises compositions and methods for activating a humoral and/or cellular immune response in an animal or human, said method comprising administering one or more chimeric antigens of the present invention.

Following cloning and expression, the chimeric antigen is evaluated for its efficacy in generating an immune response. Evaluation involves presenting the chimeric antigen to DCs ex vivo or in vivo. The DCs are evaluated for the binding and internalization of the chimeric antigens. The naïve DCs loaded with the chimeric antigen are presented to T-lymphocytes and evaluated for the production of interferon-γ as a marker of a T cell response. Specifically, in the ex vivo situation, monocytes are isolated from peripheral blood and differentiated to DCs. DCs bind, internalize, process and present antigen to naive autologous T-lymphocytes. The T cells, which recognize the processed antigens presented by DCs, are activated into effector cells, e.g. helper T cells or cytotoxic T-lymphocytes. Activation of the T cells by the dendritic cells is then evaluated by measuring markers, e.g. interferon-γ levels, by a known procedure [e.g., Berlyn, et al. (2001) Clin. Immunol. 101(3):276-283]. An increase in the percentage of T cells that produce interferon-γ by at least 50% over background predicts efficacy in vivo. In preferred embodiments, the percentage increase is at least 55%, 60%, 65%, 70%, 75%, 80%, 90% or 100%. In the case of the in vivo situation, the chimeric antigen is directly introduced parenterally in the host, where available dendritic and other antigen-processing cells have the capacity to interact with all antigens and process them accordingly.

G. Combination Therapy

Another aspect of the invention provides compositions for treating viral infections comprising a chimeric antigen and an antiviral agent. The invention also provides methods of treating viral infections comprising administering a chimeric antigen and an antiviral agent, either concurrently or sequentially.

The use of a chimeric antigen in combination with an antiviral agent, such as a nucleoside analogue, may prove to be highly efficacious in inducing sustained responses in the treatment of subjects suffering from chronic hepatitis C. The mechanisms of action of the two agents used in combination may produce synergistic effects in treatment of hepatitis C subjects. For example, a combination of an HCV antiviral such as ribavirin along with the HCV chimeric antigens described herein will produce antigen-specific cellular as well as humoral immune response and thus clear HCV infection in chronically infected subjects.

H. Methods of Preparing Chimeric Antigens

One aspect of the invention provides methods for producing a chimeric antigen comprising (a) providing a microorganism or cell line (or cell), preferably a eukaryotic, more preferably, a non-mammalian microorganism or cell line (or cell), that comprises a polynucleotide encoding a chimeric antigen; and (b) culturing said microorganism or cell line (or cell) under conditions whereby the chimeric antigen is expressed. Preferably, the microorganism or cell line (or cell) is a yeast, a plant cell line (or cell) or an insect cell line (or cell). More preferably, the cell line (or cell) is an insect cell line (or cell) selected from the group consisting of Sf9, Sf21, ExpresSF⁺®, Drosophila S2, and High Five™ cell lines or cells.

The present invention uses established recombinant DNA technology for producing the fusion proteins of selected antigen(s) and the TBD that are necessary in the practice of the invention. Fusion protein constructs are generated at the DNA level incorporating specific restriction enzyme sites, which are exploited in incorporating the desired DNA fragment into expression vectors, and used to express the desired fusion proteins in a heterologous expression system. As used herein, the term “vector” denotes plasmids that are capable of carrying the DNA, which encode the desired protein(s). The plasmid vectors used in the present invention include, but are not limited to, pFastBac HTa and the corresponding recombinant “BACMIDS” (bacterial artificial chromosomes) generated in DH10Bac™ E. coli (Invitrogen). It is possible to mobilize the ORF of the desired proteins and produce other recombinant plasmids for expression of the proteins in other systems, (bacterial or mammalian), in addition to the Bac-to-Bac® baculovirus expression system (Invitrogen), employed in the present invention. The term “expression” is used to mean the transcription of the DNA sequence into mRNA, the translation of the mRNA transcript into the fusion protein.

This is achieved by the transposition of the gene of interest into bacmids, transfected into Sf9 insect cells and producing recombinant baculovirus. These are used to infect Sf9 or High Five™ insect cells, which produce the protein of interest. The recombinant proteins produced may have an N-terminal 6×His tag, which is exploited in the purification of the proteins by using Ni-NTA Agarose (Qiagen, Hilden, Germany). The proteins may also have an N-terminal rTEV protease or other cleavage site cloned in. The Ni-purified protein is subjected to digestion with, for example, rTEV protease (Invitrogen), which also has an N-terminal 6×His tag. Following the protease digestion, the mixture can be loaded on to a Ni-NTA agarose column and the pure protein can be washed out, while the 6×His tagged fragments will be bound to the column. This method of purification is standard procedure and one skilled in the art would be able to understand the methodology without further explanation.

Cloning and expression of the DNA sequences, which encode the viral antigen and the Fc fragment of the murine monoclonal antibody to generate the chimeric antigen, can be achieved through two approaches. The first approach involves cloning the two proteins as a fusion protein, while the second approach involves incorporating specific “bio-linkers” such as biotin or streptavidin in either of the molecules, purifying them separately and generating the chimeric antigen.

In an exemplary embodiment, the hybridoma 2C12, which produces a monoclonal antibody against the Hepatitis B virus surface antigen, was used as a source of the total RNA for the murine immunoglobulin G. Total RNA was isolated and used to clone the murine Fc fragment. Specifically, the total RNA from a hybridoma cell that expresses murine IgG is isolated using Trizol® reagent (Invitrogen/Gibco BRL, product catalog number 10551-018, 10298-016; a monophasic solution of phenol and guanidine isothiocyante, as described in U.S. Pat. No. 5,346,994). The mRNA was purified from total RNA by affinity chromatography on an oligo-dT column (Invitrogen/Gibco BRL, product catalog number 15939-010). A complementary DNA (cDNA) was produced using reverse transcriptase in a polymerase chain reaction. The oligonucleotide primers were designed to add unique restriction enzyme recognition sites to facilitate cloning. This cDNA was cloned using the Bac-to-Bac® baculovirus expression system (Invitrogen/Gibco BRL, product catalog number 15939-010).

The baculovirus system, preferentially, is used because not only are large amounts of heterologous proteins produced, but also because post-translational modifications, such as phosphorylation and glycosylation, of proteins occur within the infected insect cell. In this expression system, the DNA can be cloned into vectors called pFastBac™ (Invitrogen/Gibco BRL, product catalog number 15939-010). In the Bac-to-Bac® system, the generation of recombinants is based on site-specific transposition with the bacterial transposon Tn7. The gene of interest is cloned into pFastBac®, which has mini-Tn7 elements flanking the cloning sites. The plasmid is transformed into Escherichia coli strain DH10Bac™ (Invitrogen/Gibco BRL, product catalog number 10361-012), which has a baculovirus shuttle plasmid (bacmid) containing the attachment site of Tn7 within a LacZ gene, Transposition disrupts the LacZ gene so that only recombinants produce white colonies and are easily selected for. The advantage of using transposition in E. coli is that single colonies contain only recombinants so that plaque purification and screening are not required. The recombinant bacmids are transfected in insect cells to generate baculoviruses that express recombinant proteins.

The Bac-to-Bac® baculovirus expression system is commercially available from Invitrogen and the procedures used are as described in the company protocols, available, for example, at www.invitrogen.com. The gene of interest is cloned into, for example, pFastBac HTa donor plasmid and the production of recombinant proteins is based upon the Bac-To-Bac™ baculovirus expression system (Invitrogen).

In the next step, the pFastBac HTa donor plasmid containing the gene of interest is used in a site-specific transposition in order to transfer the cloned gene into a baculovirus shuttle vector (bacmid). This is accomplished in E. coli strain DH10Bac™. The recombinant pFastBac HTa plasmids with the gene of interest are transformed into DH10Bac™ cells for the transposition to generate recombinant bacmids.

Recombinant bacmids are isolated by standard protocols (Sambrook, supra); the DNA sample was used for transfections.

In order to produce baculoviruses, the bacmid is transfected into Sf9 insect cells. Following transfection, the cells are incubated under appropriate conditions and the medium containing baculovirus is collected and stored.

Once production of baculovirus and the expression of protein have been confirmed, the virus stock is amplified to produce a concentrated stock of the baculovirus that carry the gene of interest. It is standard practice in the art to amplify the baculovirus at least two times, and in all protocols described herein this standard practice was adhered to. After the second round of amplification, the concentration of the generated baculovirus can be quantified using a plaque assay according to the protocols described by the manufacturer of the kit (Invitrogen). The most appropriate concentration of the virus to infect insect cells and the optimum time point for the production of the desired protein is generally also established.

DNA encoding proteins of interest are generated by PCR with oligonucleotide primers bearing unique restriction enzyme sites from plasmids that contain a copy of the entire viral genome and cloned with the Fc DNA as a fusion protein. This chimeric protein is purified by Ni-NTA, lectin, protein A or protein G affinity chromatography or other standard purification methods known to those skilled in the art.

The second approach for linking the IRD and TBD involves incorporating specific “bio-linkers” such as biotin or avidin (e.g., streptavidin) in either of the molecules, purifying them separately and generating the chimeric antigen. The viral antigens of interest are cloned into plasmids that control the expression of proteins by the bacteriophage T7 promoter. The recombinant plasmid is then transformed into an E. coli strain, e.g. BL21 (DE3) Codon Plus™ RIL cells (Stratagene, product catalog number 230245), which has production of T7 RNA polymerase regulated by the lac repressor. The T7 RNA polymerase is highly specific for T7 promoters and is much more processive (−8 fold faster) than the E. coli host's RNA polymerase. When production of T7 RNA polymerase is induced by isopropylthio-β-D-galactoside (IPTG), the specificity and processivity of T7 RNA polymerase results in a high level of transcription of genes under control of the T7 promoter. In order to couple two proteins together, the tight binding between biotin and avidin (e.g., streptavidin) is exploited. In E. coli, the BirA enzyme catalyzes the covalent linkage of biotin to a defined lysine residue in a specific recognition sequence. The murine Fc fragment is expressed in the baculovirus system, as described above, as a fusion protein with avidin. These two proteins can be mixed to form a dimeric protein complex by biotin-streptavidin binding.

The practice of the present invention employs, unless otherwise indicated, conventional techniques of molecular biology, microbiology, recombinant DNA, and immunology, which are within the skill of the art. Such techniques are explained fully in the literature. See e.g., Sambrook, supra; and Ausubel, supra.

I. Articles of Manufacture and Kits

Another aspect of this invention provides an article of manufacture that comprises a container holding a composition, comprising a chimeric antigen, that is suitable for injection or reconstitution for injection in combination with printed labeling instructions providing a discussion of how to administer the composition parenterally, e.g. subcutaneously, intramuscularly, intradermally, nasally or intravascularly. The composition can be contained in any suitable container that will not significantly interact with the composition and can be labeled with the appropriate labeling that indicates it will be for parenteral use. Associated with the container can be the labeling instructions consistent with the method of treatment as described hereinbefore. The container that holds the composition of this invention can be a container having a liquid composition suitable for injection. The container can be adapted for access by a syringe needle. The article of manufacture can include an appropriate needle and a syringe for injection so that a patient, doctor, nurse, or other practitioner can administer the chimeric antigen. Alternatively, the composition can be a dry or concentrated composition containing a soluble version of the chimeric antigen, to be combined or diluted with an aqueous or nonaqueous vehicle to dissolve or suspend the composition. Alternatively, the container may have a suspension in a liquid or may be an insoluble version of the salt for combination with a vehicle in which the insoluble version will be suspended. Appropriate containers are discussed in Remington, supra, pages 788-789, 805, 850-851 and 1005-1014

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and can also indicate directions for either in vivo or ex vivo use, such as those described above. Directions and or other information can also be included on an insert which is included with the kit.

### V. EXAMPLES

The following non-limiting examples provide further illustration of the invention.

### Example 1

### Materials and Methods

### Materials

The TBD used in the Chimigen3 molecules described in these examples (and, for convenience, referred to in the examples as “TBD”) is derived from the Hybridoma 2C12, which produces a murine HBsAg-specific mAb and which was licensed from the Tyrrell laboratory through the University of Alberta, Edmonton, Alberta, Canada. The plasmid pCV-H77C containing the DNA encoding the HCV antigens was obtained from the Tyrrell laboratory at the University of Alberta.

The pFastBac-HTa cloning vector, insect cell line Sf9, Cellfectin® reagent, phosphate buffered saline (PBS), Platinum pa DNA polymerase, TRizol reagent, Superscript First-Strand Synthesis reverse transcriptase, X-gal, isopropyl-β-D-thiogalactopyranoside (IPTG) and fetal bovine serum (FBS) were purchased from Invitrogen (Carlsbad, Calif., USA).

Insect cell growth and expression medium ESF 921 was purchased from Expression Systems (Woodland, Calif., USA). Restriction enzymes EcoR I, Spe I, Hind III, Rsr II, Ava II and Not I were purchased from New England Biolabs (Ipswich, Mass., USA).

Viral stocks were titered using the Expression Systems Baculovirus Titering Assay. IgG_{2A}-PE (BD Biosciences, San Diego, Calif., USA) was diluted 1:10 and used as an isotype control. Baculovirus titer was determined using FACS acquisition and analysis. A Becton Dickinson Biosciences FACSCalibur3 (four-color, dual-laser) acquired cells and CELLQuest Pro3 software (BD Biosciences) was used to analyze the data. A Microsoft Excel spreadsheet was provided by Expression Systems to input data and determine the viral titer based on a standard curve. Purifications were performed with Ni-NTA Superflow3 (Qiagen, Hilden, Germany) and Toyopearl Super Q3 650C (Tosoh Biosciences, Grove City, Ohio, USA).

The 30% acrylamide solution for making sodium dodecyl sulfate polyacrylamide gel electrophoresis (SDS PAGE) gels was purchased from Bio-Rad (Hercules, Calif., USA). PageBlue3 stain, 5× loading buffer, PageRuler3 pre-stained protein ladder and 20× reducing agent were purchased from Fermentas (Burlington, ON, Canada).

Hybond3 ECL nitrocellulose and the ECL Western Detection kit (GE Healthcare) was used for Western Blotting.

Tween 20, hexadecyltrimethylammonium bromide (CTAB), anti-mouse IgG (Fc specific) horseradish peroxidase conjugated antibody, anti-mouse (Fab specific) horseradish peroxidase conjugated secondary antibody, goat-anti-rabbit horseradish peroxidase conjugated secondary antibody and antibiotics kanamycin, ampicillin and gentamicin were purchased from Sigma (St. Louis, Mo., USA).

The rabbit anti-NS5A, goat anti-NS3, and goat anti-NS4 polyclonal antibodies and mouse anti-NS5A monoclonal antibody were obtained from Abcam (Cambridge, Mass., USA). The 6×His horseradish peroxidase conjugated monoclonal antibody was purchased from Clontech (Palo Alto, Calif., USA).

Slide-a-lyzer3 cassettes and Micro BCA3 assay kit were purchased from Pierce (Rockford, Ill., USA).

Pro-Q® Emerald 300 Glycoprotein Gel and Blot Stain Kit was purchased from Molecular Probes (Carlsbad, Calif., USA).

Leukapheresis samples from healthy donors were purchased from SeraCare Life Sciences (Oceanside, Calif., USA). Dynal Dynabeads3 for T cell negative isolation were purchased from Invitrogen (Carlsbad, Calif., USA). AIM V® medium containing L-glutamine, streptomycin sulfate (50 μg/mL), and gentamycin sulfate (10 μg/mL) was obtained from Invitrogen. Matched donor sera were obtained from the serum fraction after centrifugation of Ficoll-Hypaque blood preparations. Serum, at 50% in AIM V® medium was heat inactivated, aliquoted, and stored at −20° C. Dulbecco's phosphate buffered saline (PBS) was obtained from Invitrogen.

Conjugated monoclonal antibodies (mAbs) with the following specificities were obtained from BD Biosciences (San Diego, Calif.): CD64-fluorescein isothiocyanate (FITC), CD32-R-phycoerythrin (PE), CD 16-PE, CD206-PE-Cy5, CD80-PE, CD86-FITC, CD83-PE, CD40-FITC, CD11c-PE, CD14-FITC, CD19-FITC, CD3-FITC, CD3-PE, CD3-allophycocyanin (APC), CD8-PE-Cy5, CD4-APC, CD69-FITC, CD69-APC, HLA-ABC-FITC, HLA-DR-PE, INF-γ-PE, TNF-α-PE, grB-FITC, pfn-FITC and mouse IgG1-biotin. Biotinylated anti-6×His was obtained from Qiagen (Mississauga, Ontario, Canada). Goat anti-rabbit IgG-biotin antibody was from Jackson ImmunoResearch Laboratories (West Grove, Pa.). Murine isotype mAbs and SA-PE-Cy5 were obtained from BD Biosciences. Mixed isomer 5-(and-6)-carboxyfluoresceindiacetate, succinimidyl ester (5(6)-CFDA, SE; CFSE) was obtained from Invitrogen.

Specificity of T cells to antigens was measured with the use of specific PE-conjugated tetramers (Beckman Coulter, Mississauga, Ontario, Canada) or pentamers (ProImmune, Springfield, Va.). Pentamers used'included the HCV NS5A peptide VLSDFKTWL (SEQ ID NO:3)/HLA-A2 and the HCV NS3 peptide CINGVCWTV (SEQ ID NO:4)/HLA-A2, Tetramers used included the EBV peptide GLCFLVAML (SEQ ID NO:5)/HLA-A2, the HCV NS3 peptide KLVALGINAV (SEQ ID NO. 6)/HLA-A2 and a negative control tetramer (multiallelic).

The following cytokines were purchased from R&D Systems (Minneapolis; Minn.): interleukin-1θ (IL-1β), interleukin-4 (IL-4), interleukin-6 (IL-6), granulocyte macrophage-colony stimulating factor (GM-CSF), tumor necrosis factor-I (TNF-α), interferon-K (LPN-γ), and interferon-I (IFN-α). Cytokines were reconstituted according to the manufacturers' directions, aliquoted, and stored at −70° C. Poly IC was obtained from Sigma.

The Wave Bioreactor System23/10EH and Cellbag 10L/O were purchased from Wave Biotech (Somerset, N.J., USA)

### Methods

### Expression Plasmid Construction

pFastBacHTa-TBD, the Parent Plasmid Construct

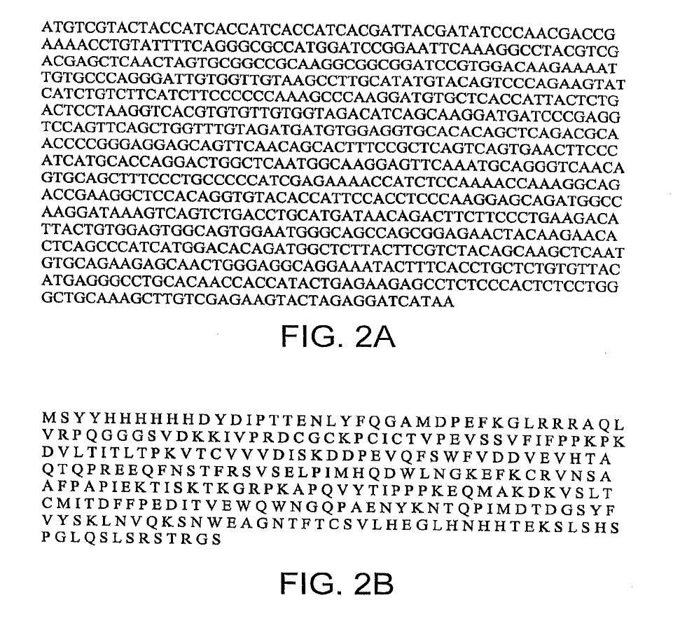

The mouse IgG1 DNA sequences encoding amino acids of C_{H}1-Hinge-C_{H}2-C_{H}³ region were generated from mRNA isolated from the hybridoma 2C12 that produces a mAb against HBV surface antigen (sAg). Total mRNA was isolated using TRizol reagent and the cDNA of the TBD was generated by RT-PCR using Superscript First-Strand Synthesis. The PCR primers contained linker sequences encoding the linker peptide —SRPQGGGS— (SEQ 11) NO:1) at the 5′ terminus, a unique Not I site at the 5′ end and\ a unique Hind III restriction site at the 3′ end. The resulting cDNA contains (5′ Not I)-linker sequence-part of C_{H}1 (VDKKI; SEQ ID NO:2)-C_{H}2-C_{H}3 (3′ Hind III). Following digestion with the respective enzymes, the fragment was ligated with pFastBac-HTa expression vector plasmid using the same restriction enzyme sites. The 5′ primer used for PCR amplification was (Sense) 5′-TGTCATTCTGCGGCCGCAAGGCGGCGGGATCCGTGGACAAGAAAATTGTGCC AGG-3′ (SEQ ID NO:7) and the 3′ primer was (antisense) 5′-ACGAATCAAGCTTTGCAGCCCAGGAGAGTGGGAGAG-3′ (SEQ ID NO:8), which contained the Not I and Hind III sites, respectively. The amplified DNA was digested with Not I and Hind III, the fragment purified by agarose gels and ligated with pFastBac-HTa expression vector plasmid digested with the same restriction enzymes to produce the expression plasmid pFastBacHTa-TBD. This product was used for the expression of the fusion protein 6×His tag-rTEV protease cleavage site-TBD. The DNA sequence and the accuracy of the open reading frame (ORF) were verified by standard sequencing methods. The nucleotide sequence (SEQ ID NO:9) of the ORF in pFastBacHTa-TBD and the amino acid sequence (SEQ ID NO:10) encoded by the ORF are shown in FIG. 2.

Construction of pFastBacHTa-gp64

For secretion, the signal sequence from the Autographa californica nuclear polyhedrosis virus (AcNPV) gp64 protein was cloned into pFastBac-HTa. Two oligonucleotides were synthesized and annealed together. The oligonucleotide sequences are 5′-GCATGGTCCATGGTAAGCGCTATTGTTTTATATGTGCTTTTGGCGGCGGCGGC GCATTCTGCCTTTGCGGATCTGCAGGTACGGTCCGATGC-3′ (SEQ ID NO:11) and 5′-GCATCGGACCGTACCTGCAGATCCGCAAAGGCAGAATGCGCCGCCGCCGCCA AAAGCACATATAAAACAATAGCGCTTACCATGGACCATGC-3′ (SEQ ID NO:12). The oligonucleotides contain a 5′ Ava II site and 3′ Rsr II site. After digestion with Ava II and Rsr II, the fragment was cloned into the Rsr II digested pFastBac-HTa, which places the gp64 signal sequence immediately upstream of the 6×His tag, to generate pFastBacHTa-gp64.

Construction of pFastBacHTa HCV NS5A Chimigen™ Vaccine Fusion Protein Expression Vector Plasmid

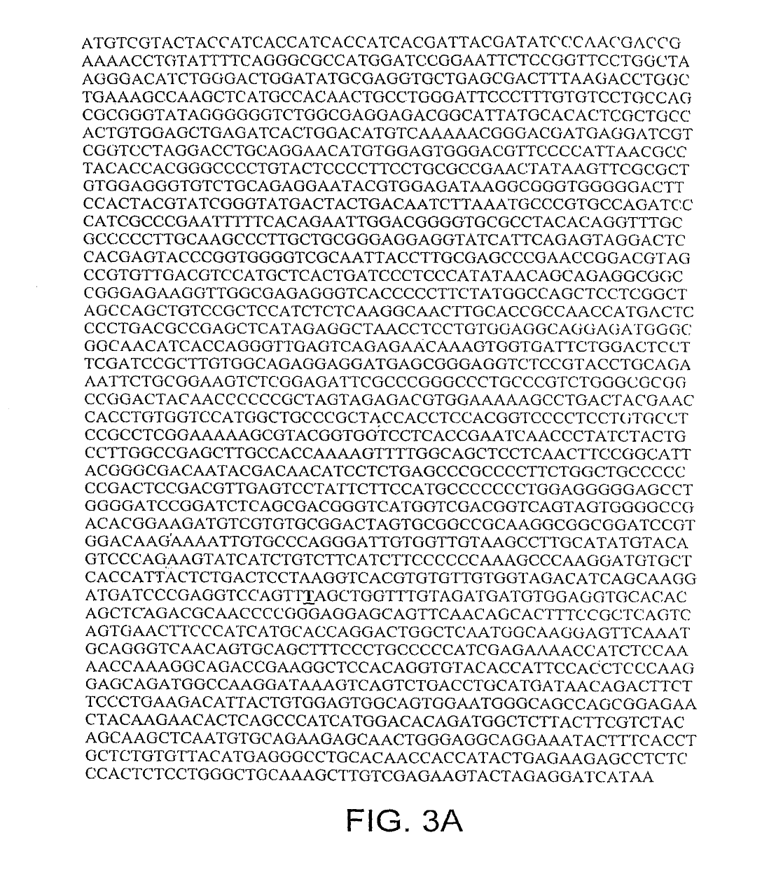

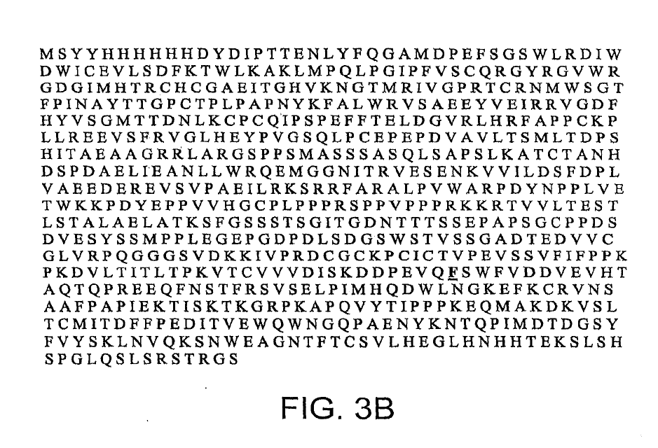

DNA encoding the HCV NS5A fragment was generated from the plasmid pCV-H77C template using PCR methodology. The 5′ primer used for the PCR was (sense) 5′-CCGGAATTCTCCGGTTCCTGGCTAAGG-3′ (SEQ ID NO:13) containing the restriction enzyme EcoR I site. The PCR primer for 3′ terminus was (antisense) 5′-GGACTAGTCCGCACACGACATCTTCCGT-3′ (SEQ ID NO:14) and contains the restriction enzyme Spe I site. Amplified DNA was digested with the respective enzymes and ligated to pFastBacHTa-TBD to generate the expression plasmid pFastBacHTa HCV NS5A Chimigen™ Vaccine (or pFastBacHTa-NS5A-TBD). The nucleotide sequence (SEQ ID NO:39) and the amino acid sequence (SEQ ID NO:40) encoded by the ORF in pFastBacHTa-NS5A-TBD are presented in FIG. 3. For NS5A alone, the NS5A fragment was ligated into EcoR I/Spe I digested pFastBac-HTa to generate pFastBacHTa-NS5A.

Construction of pFastBacHTa-gp64 NS5A Chimigen™ Vaccine Expression Plasmid for Secretion

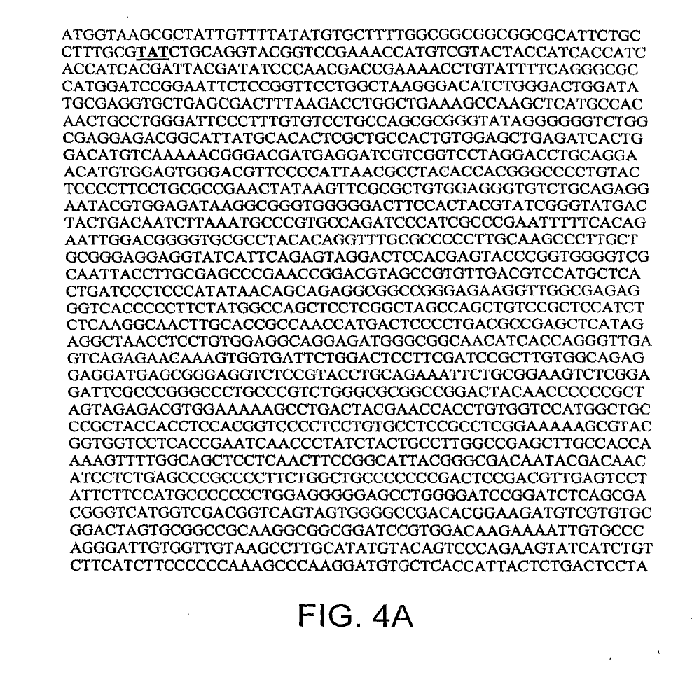

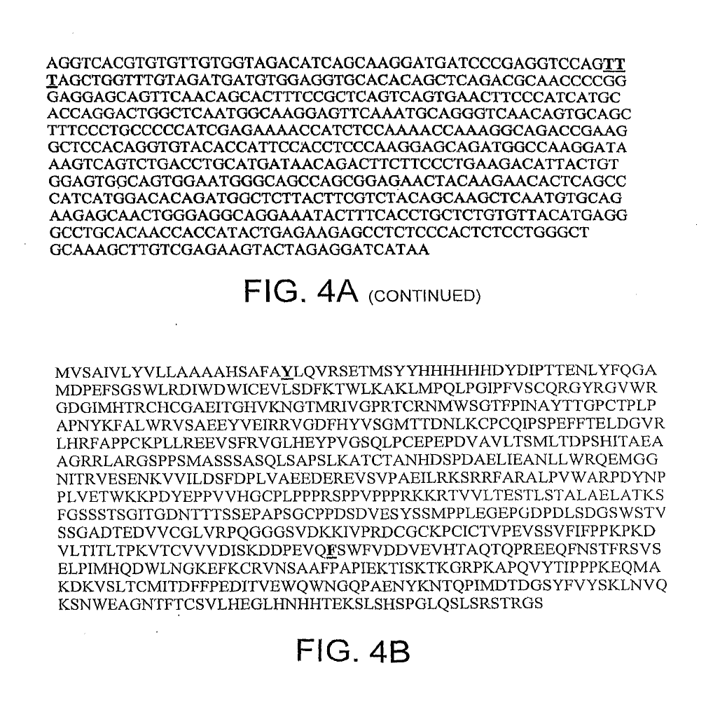

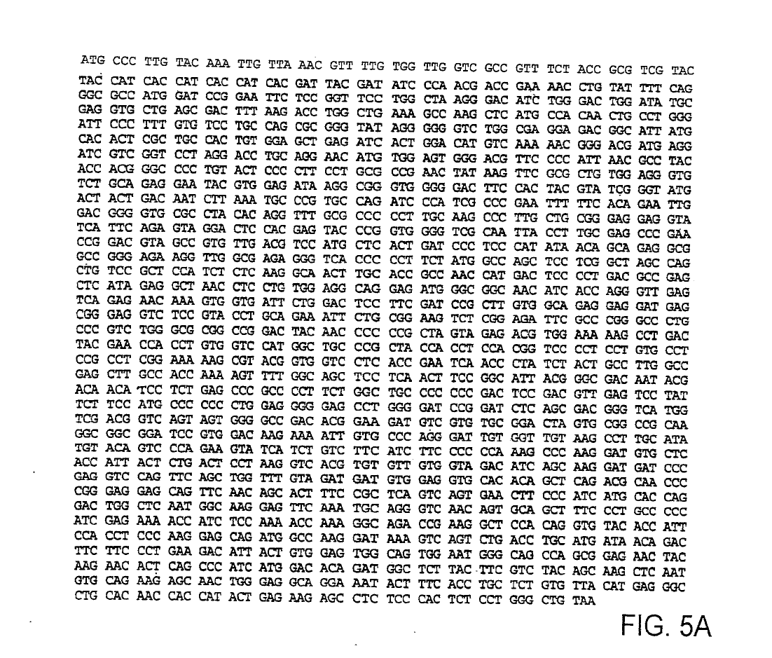

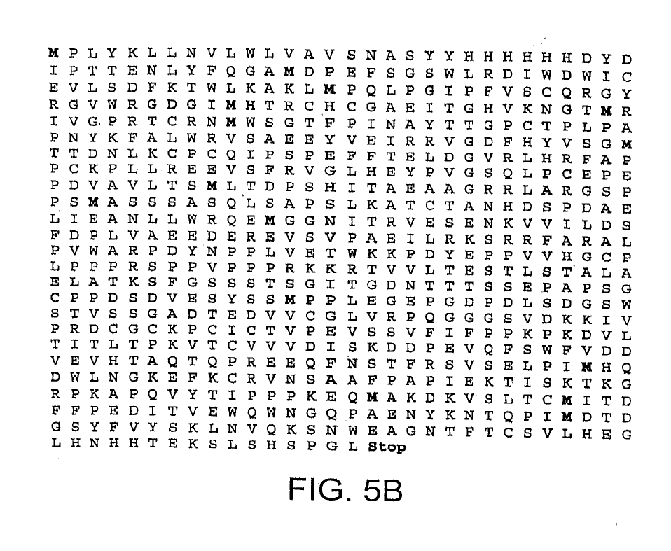

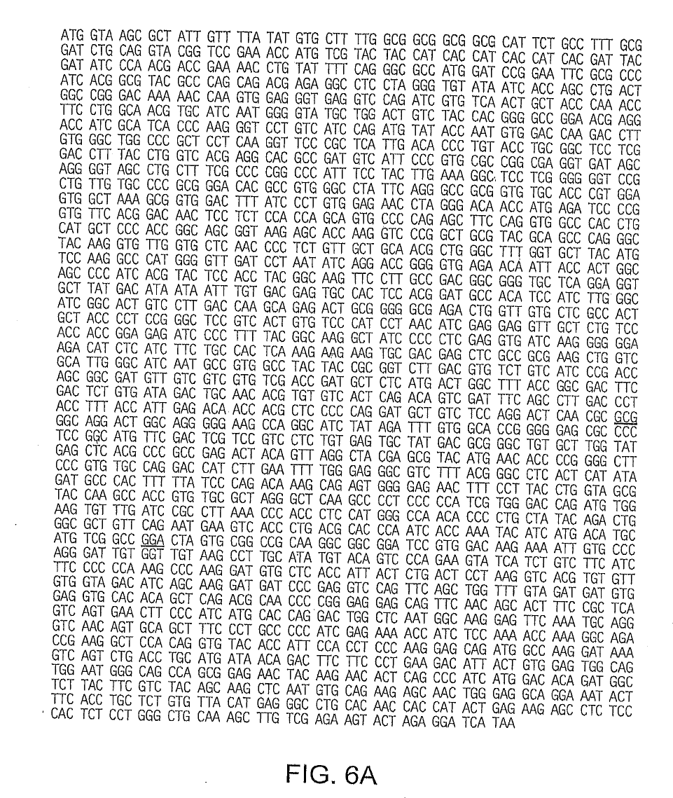

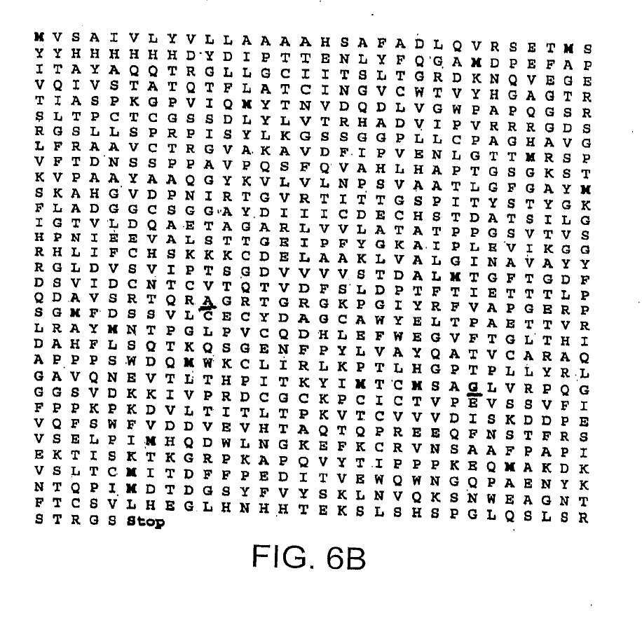

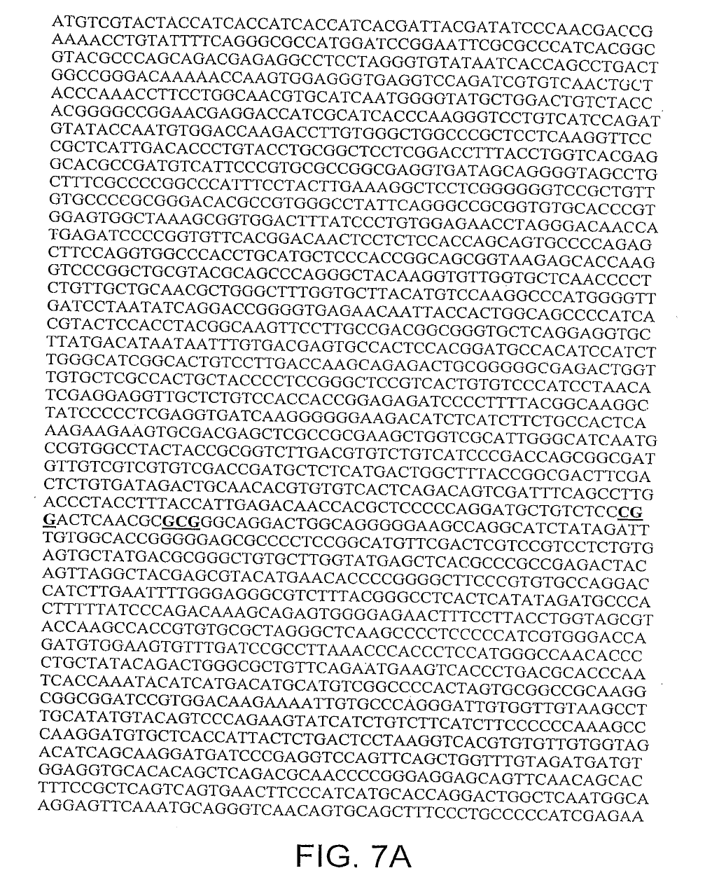

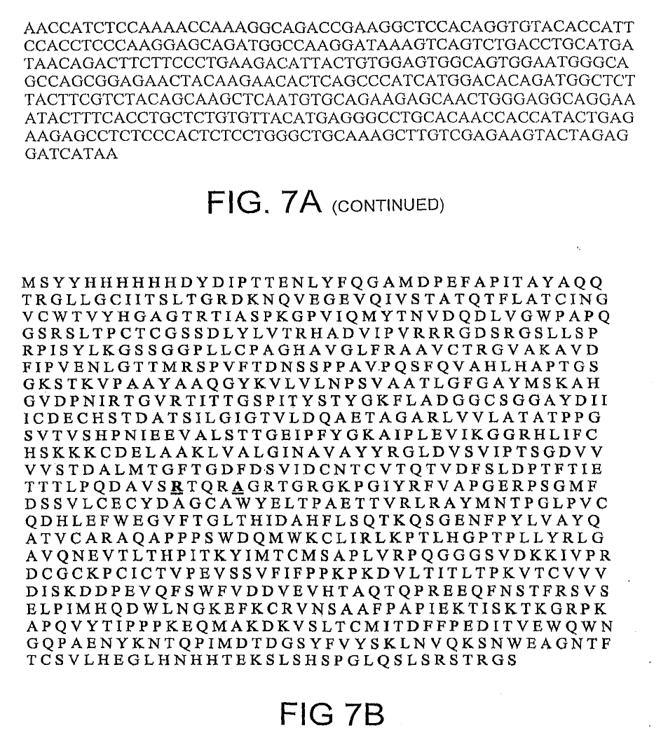

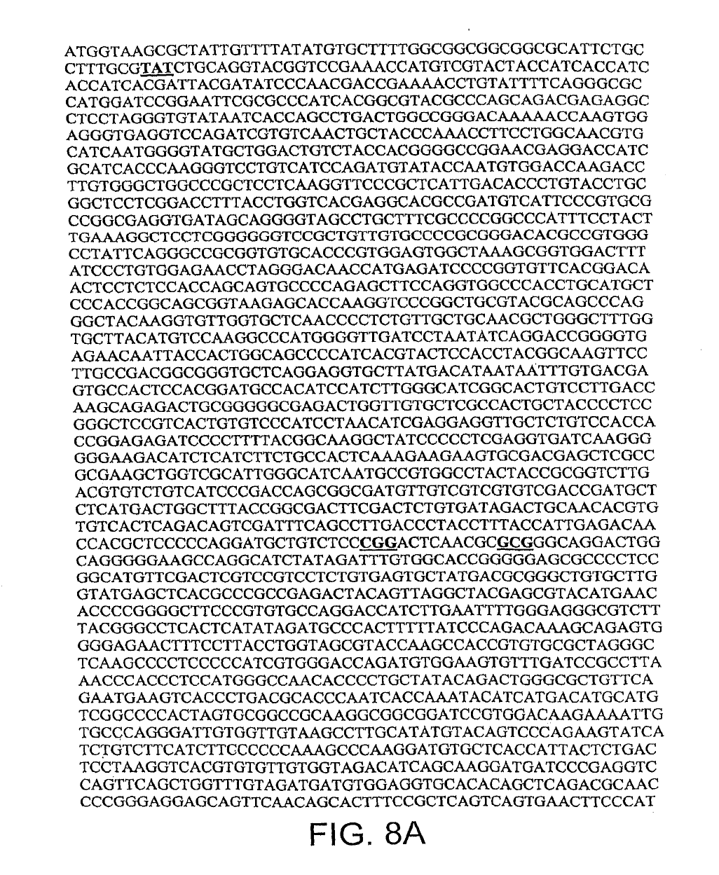

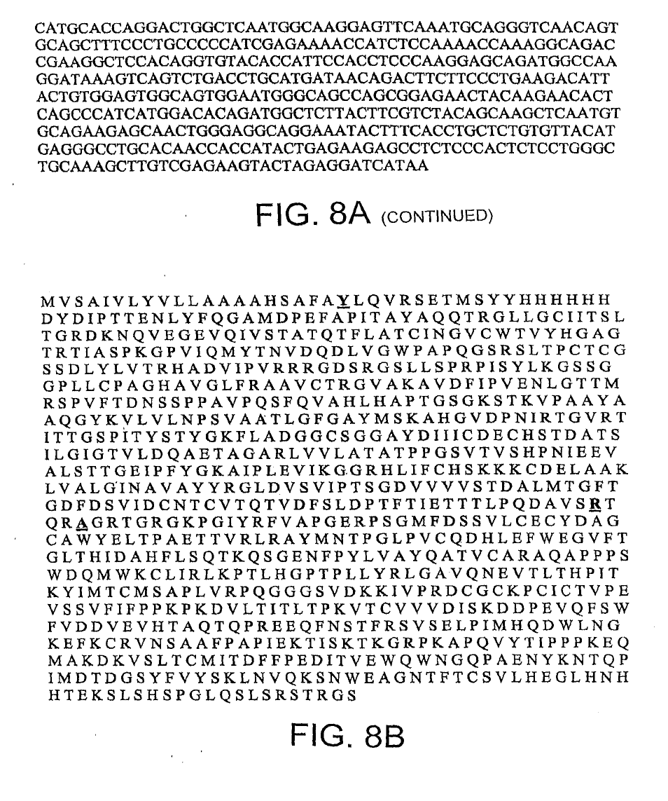

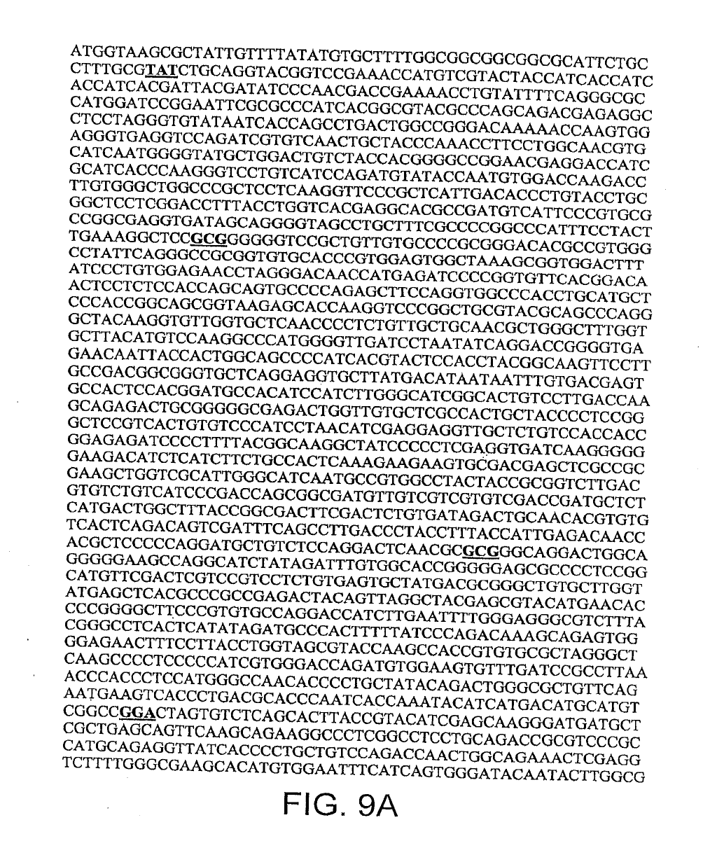

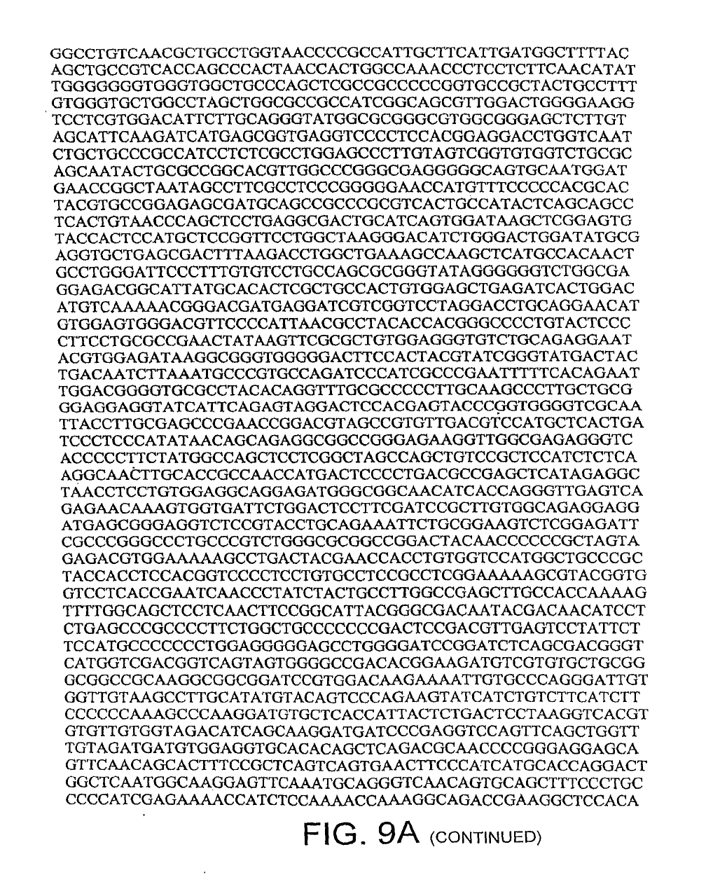

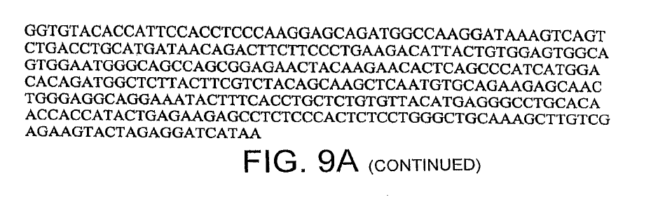

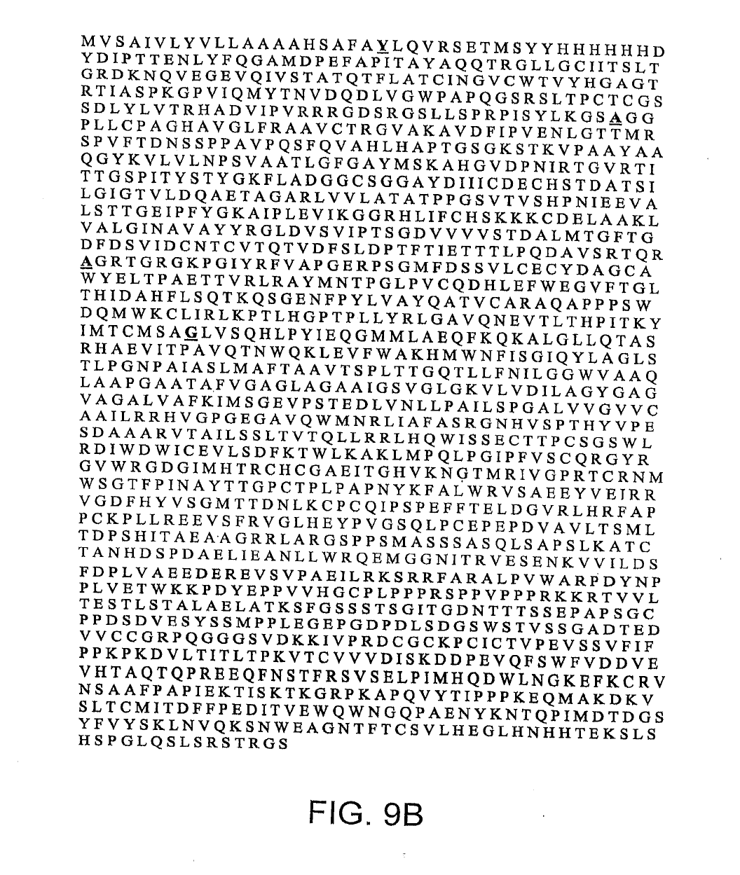

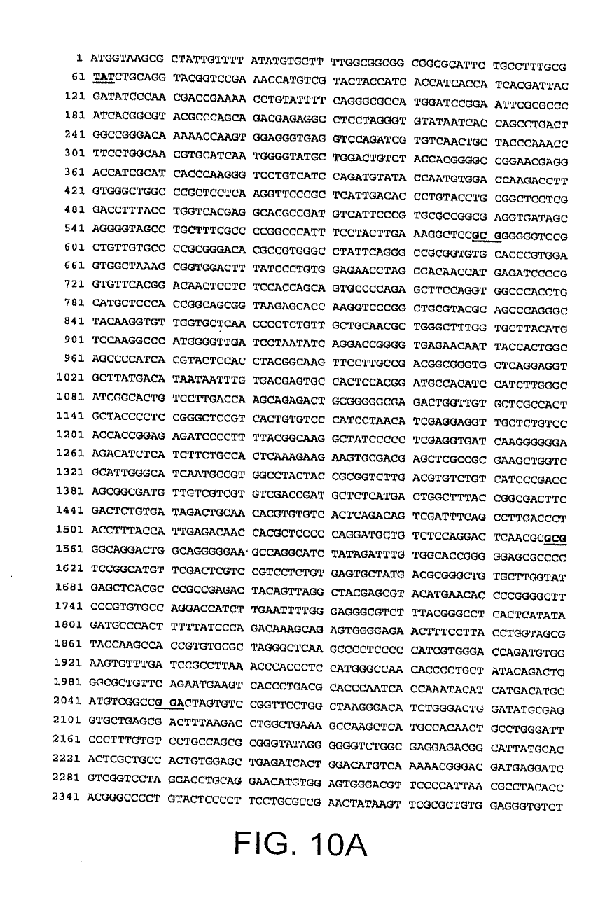

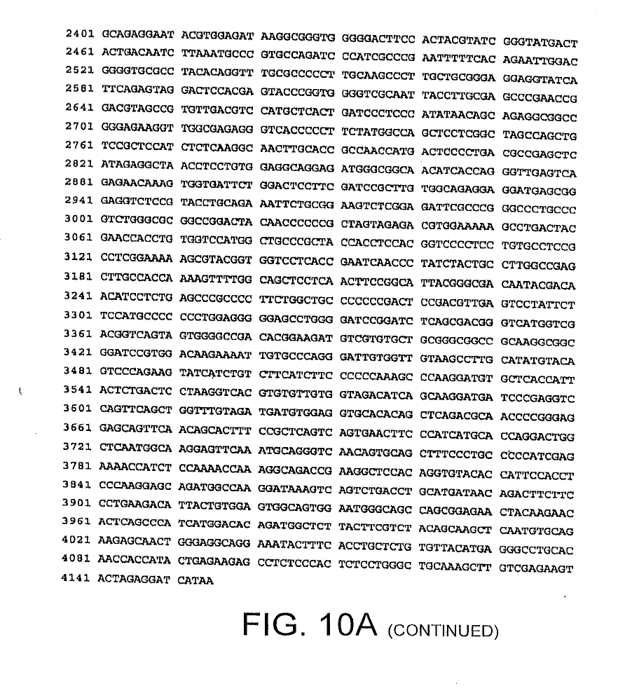

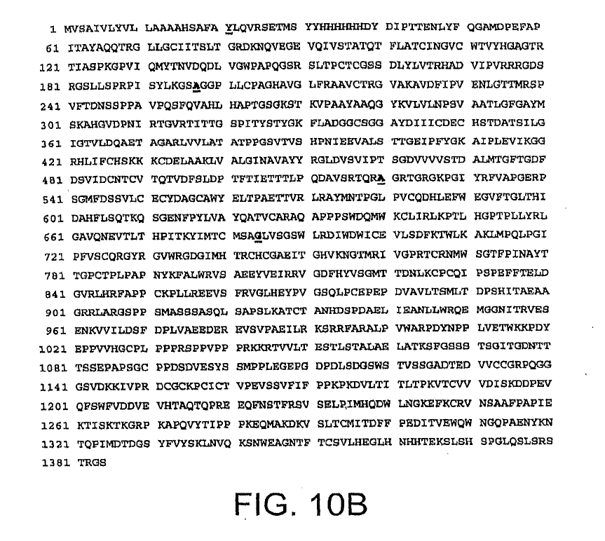

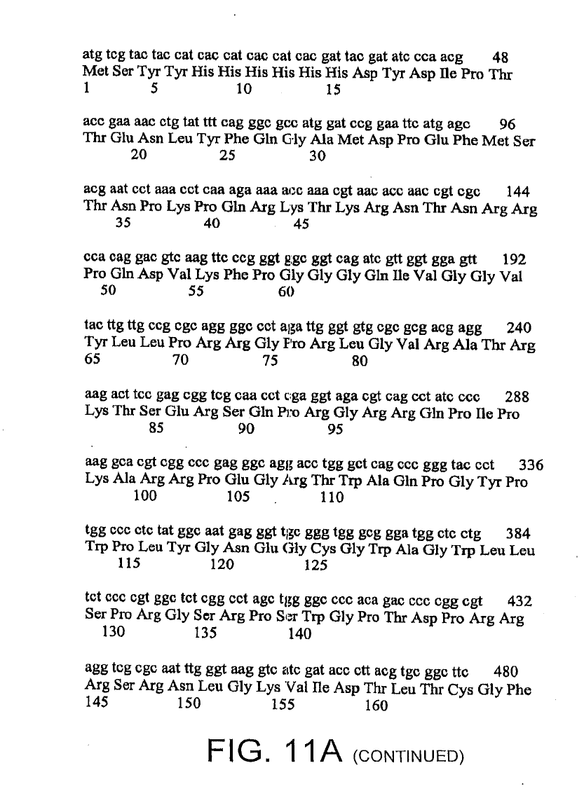

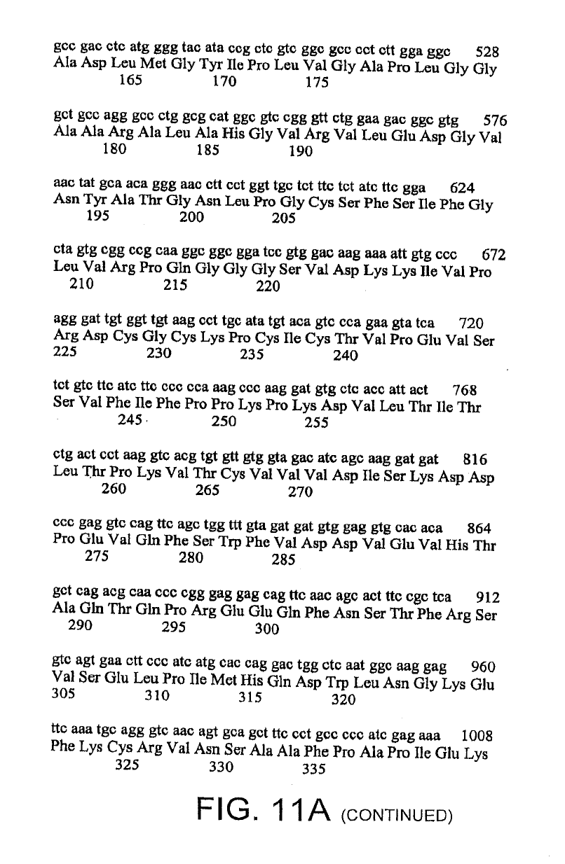

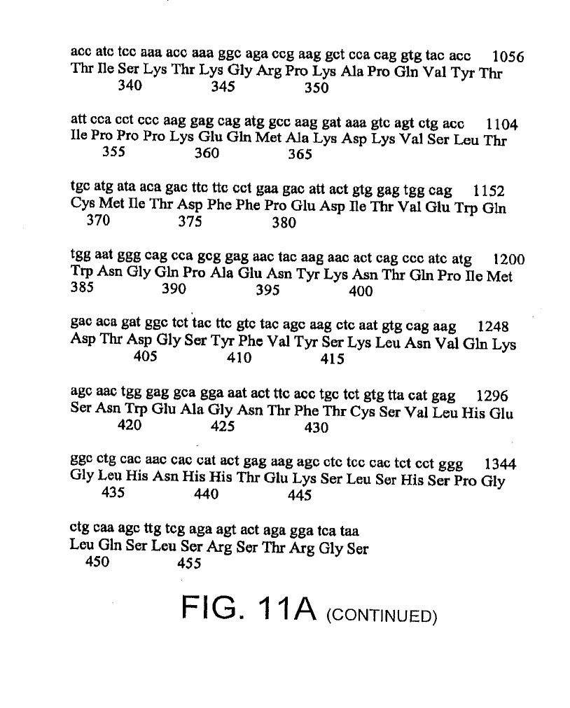

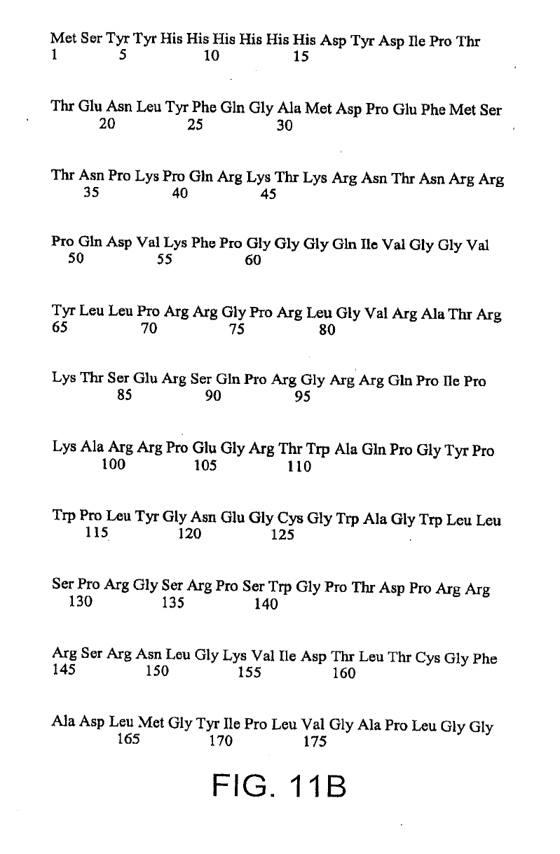

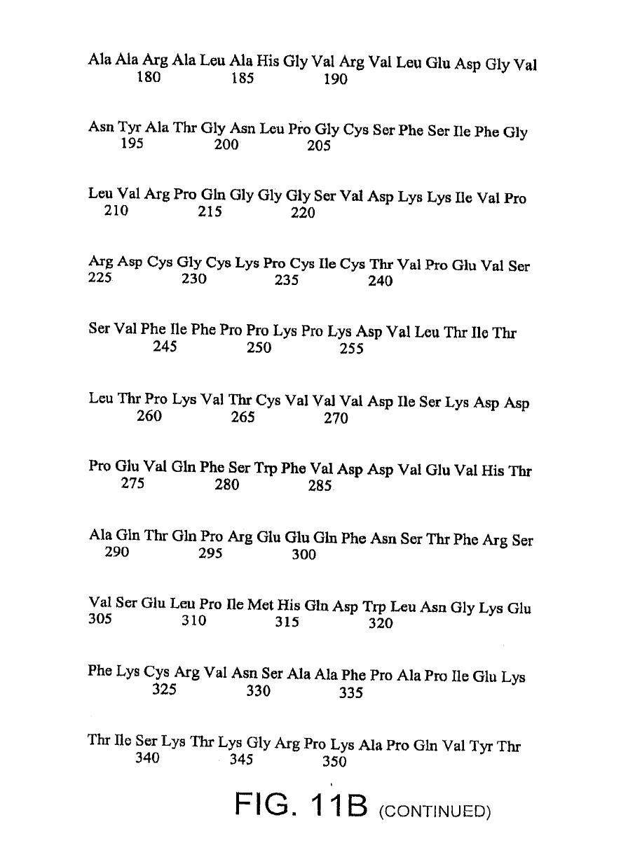

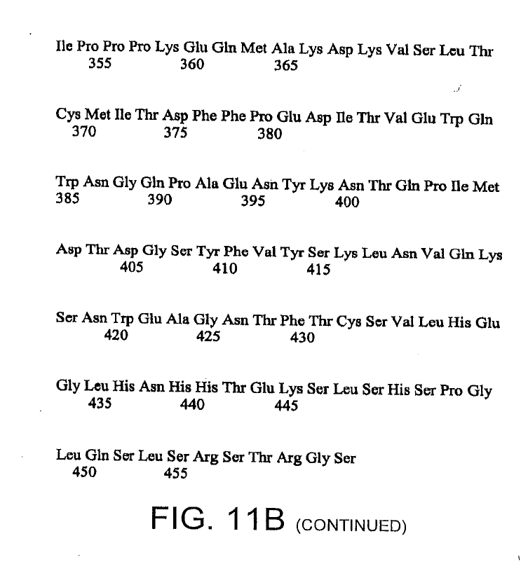

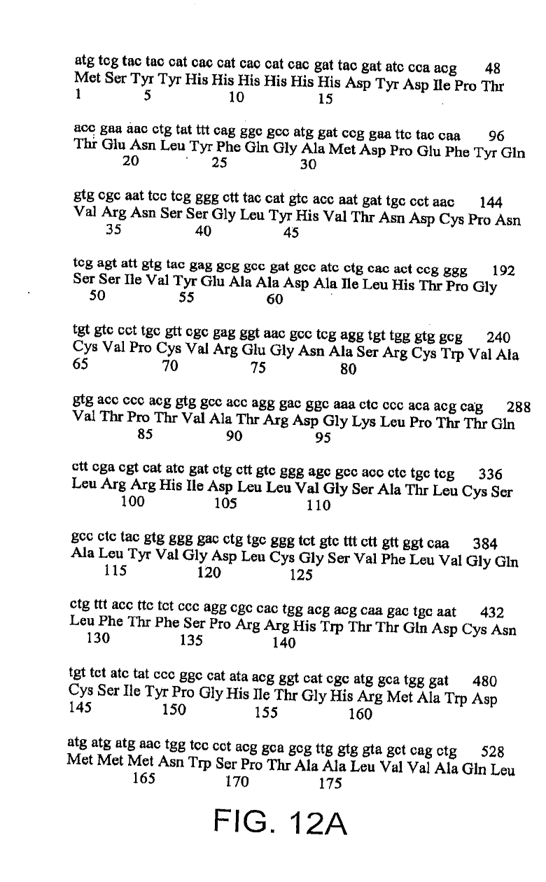

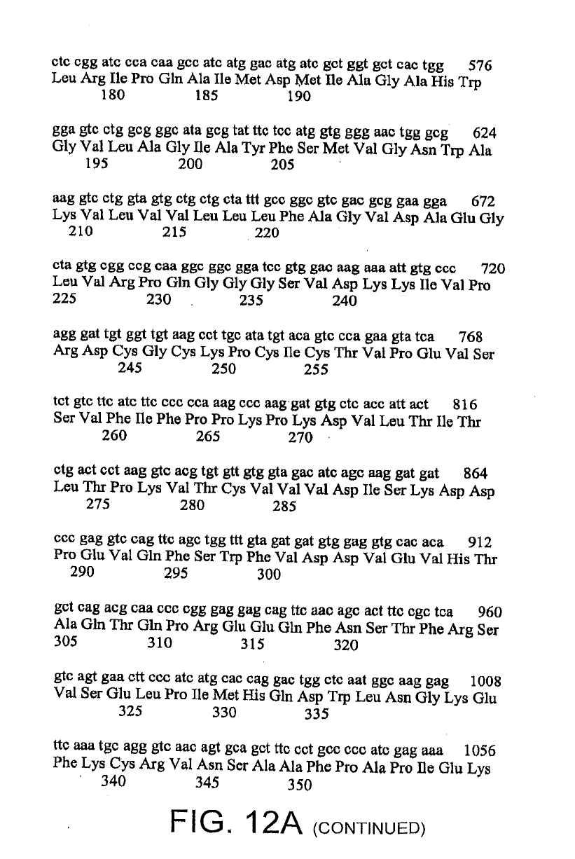

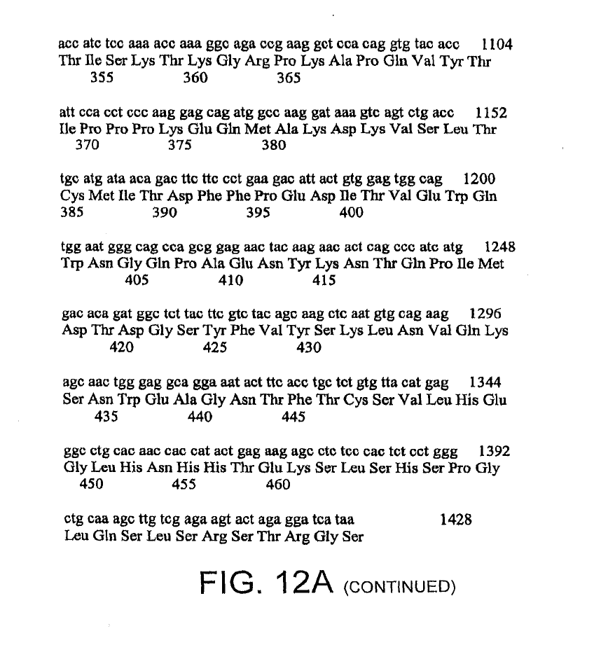

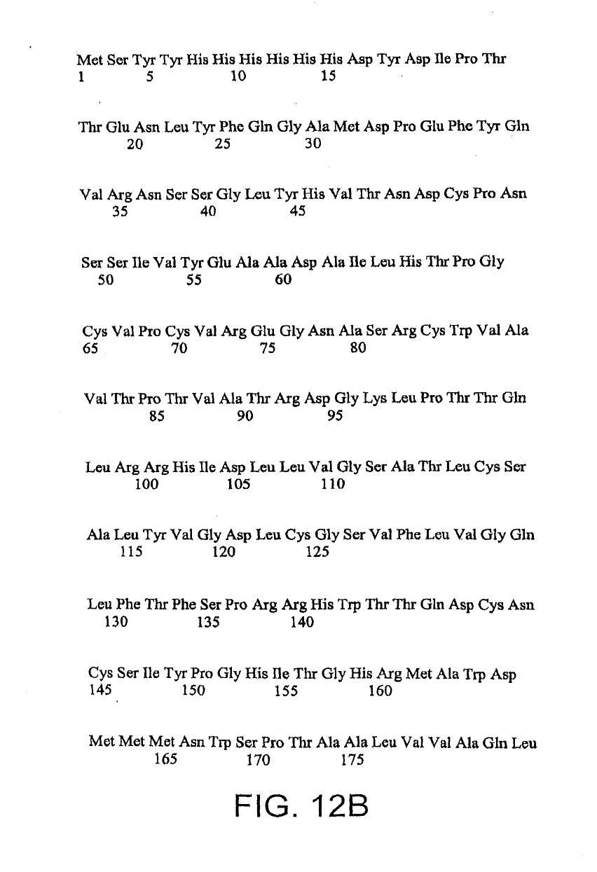

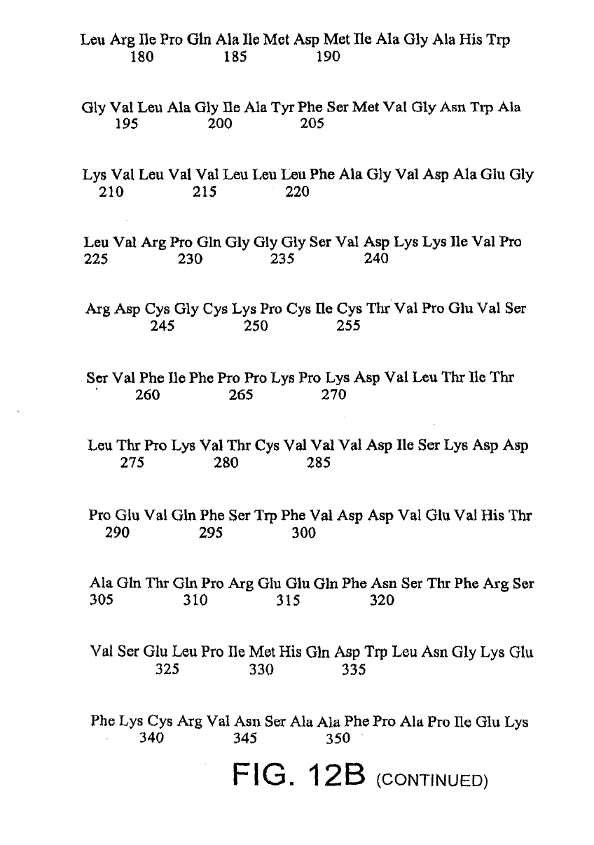

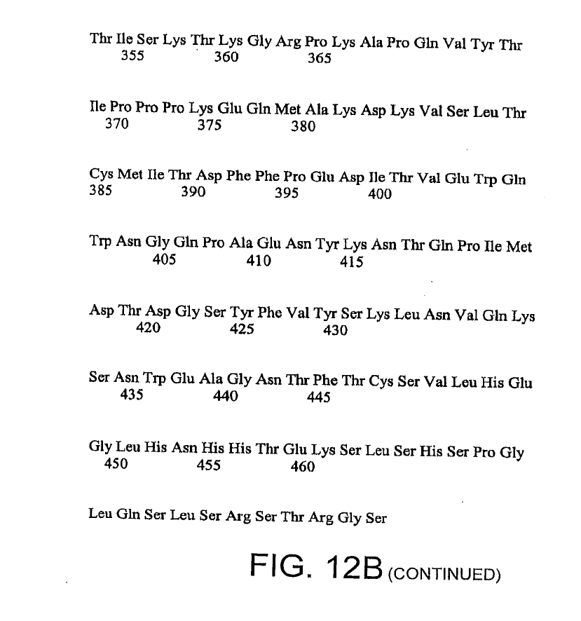

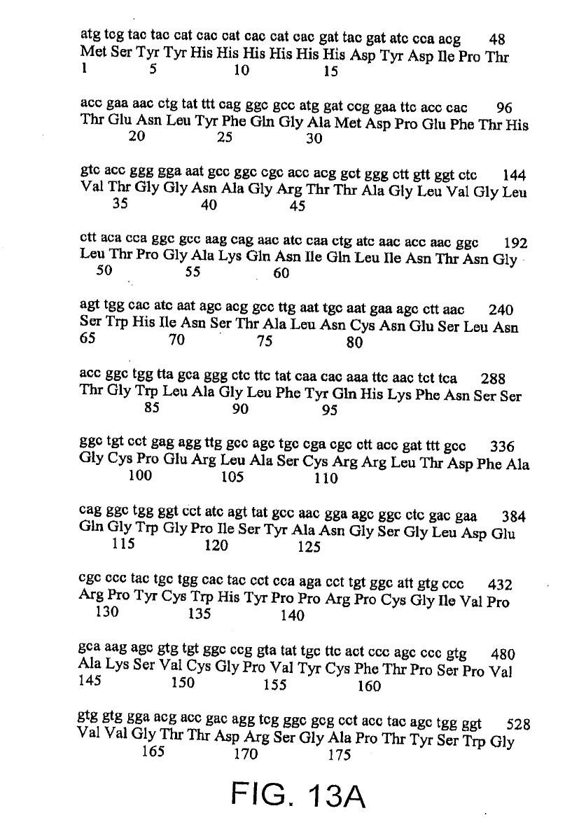

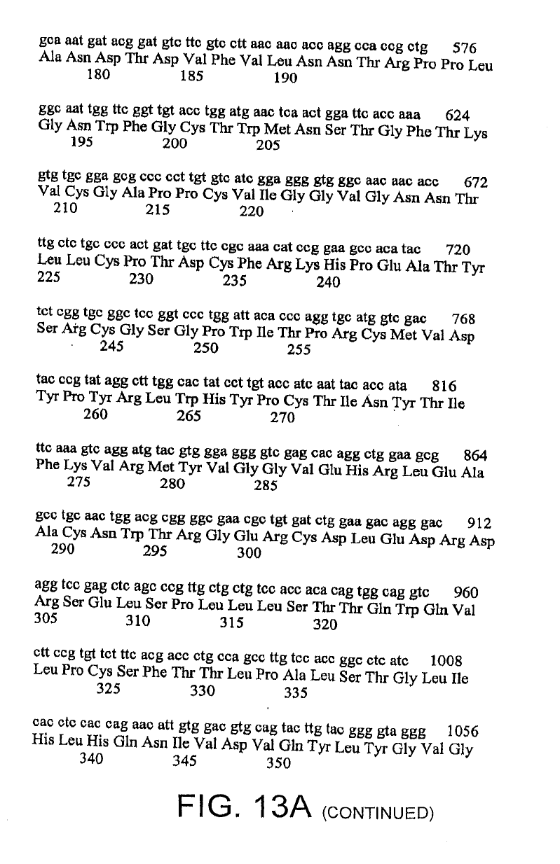

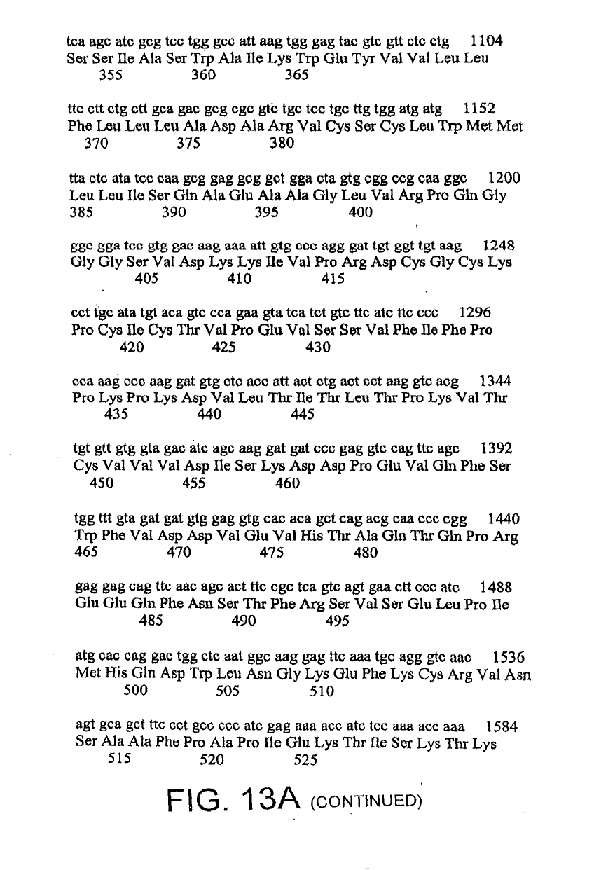

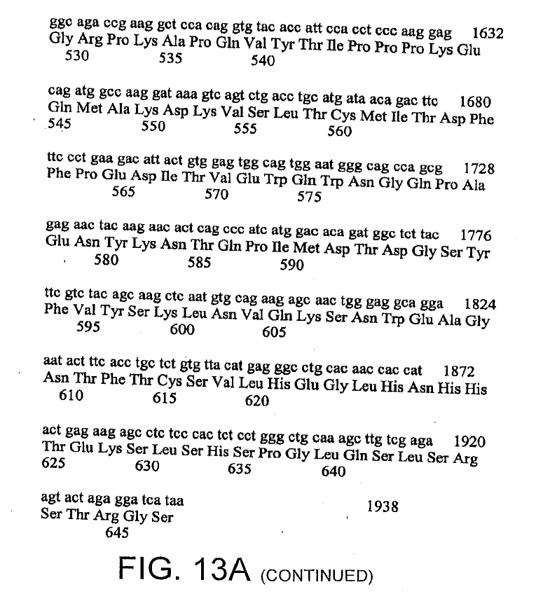

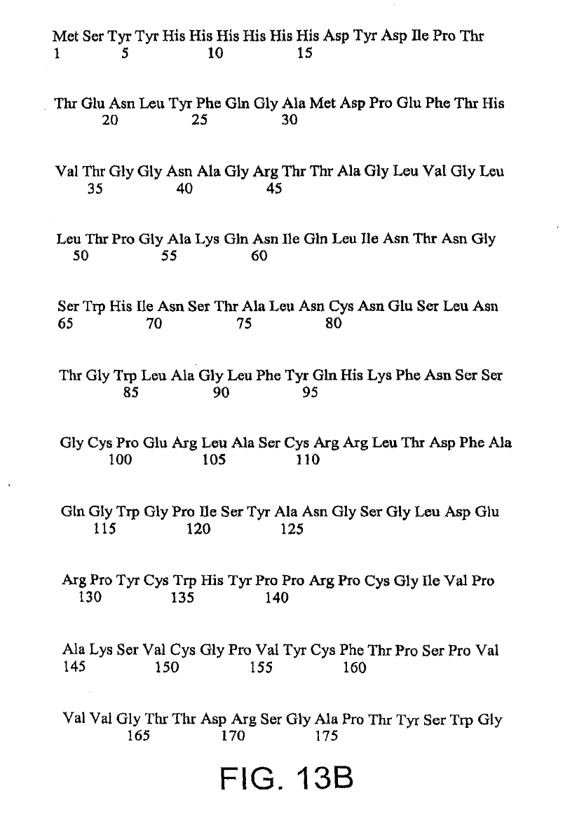

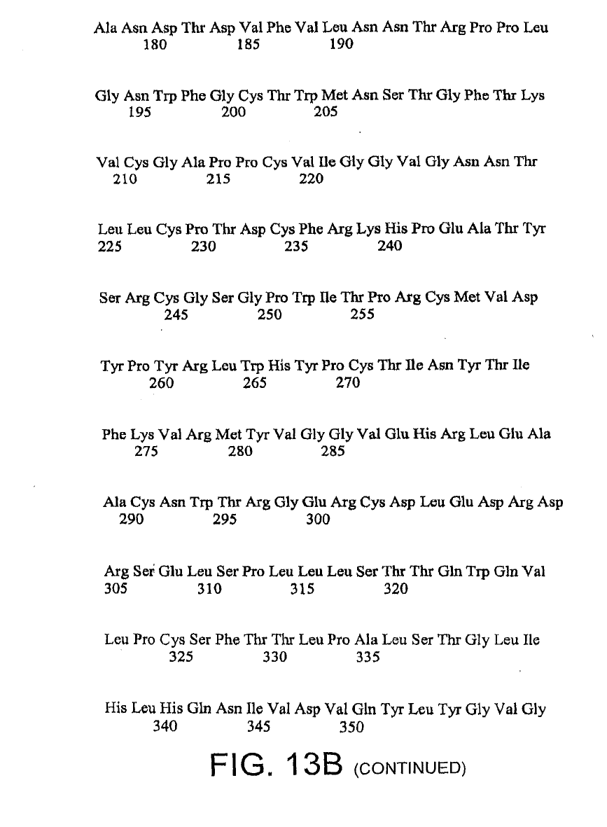

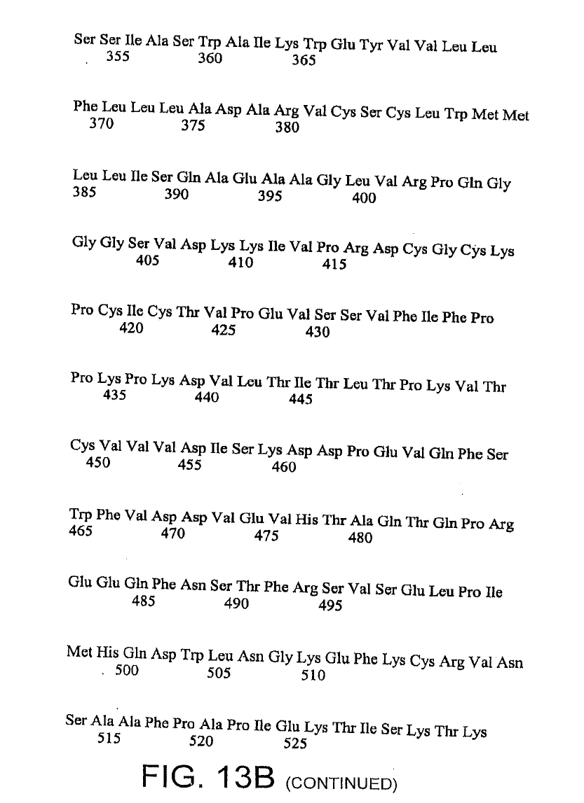

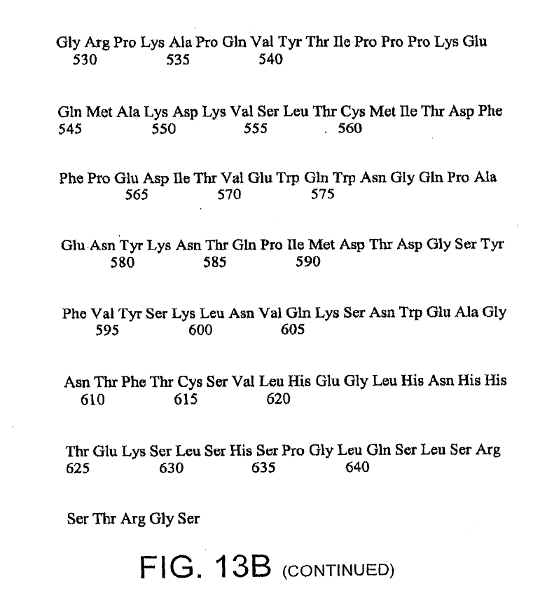

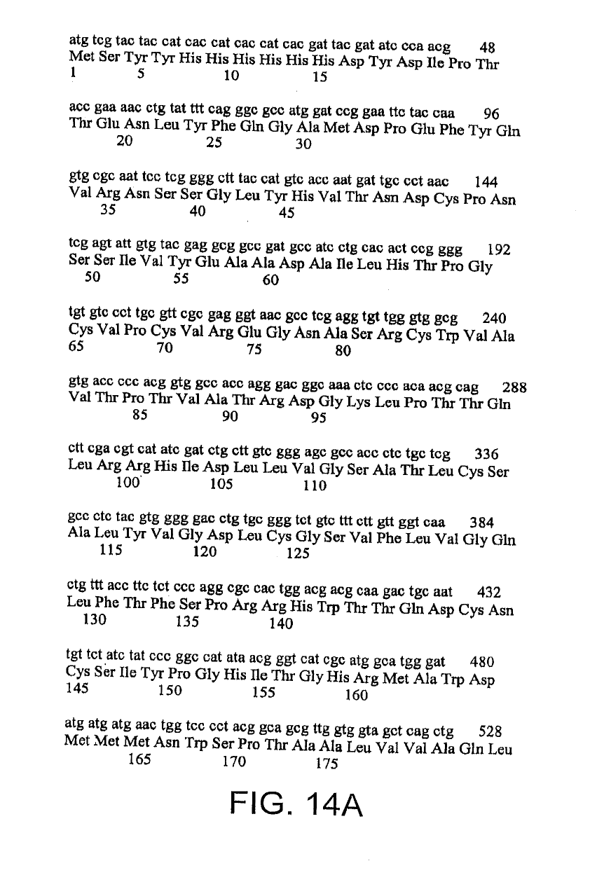

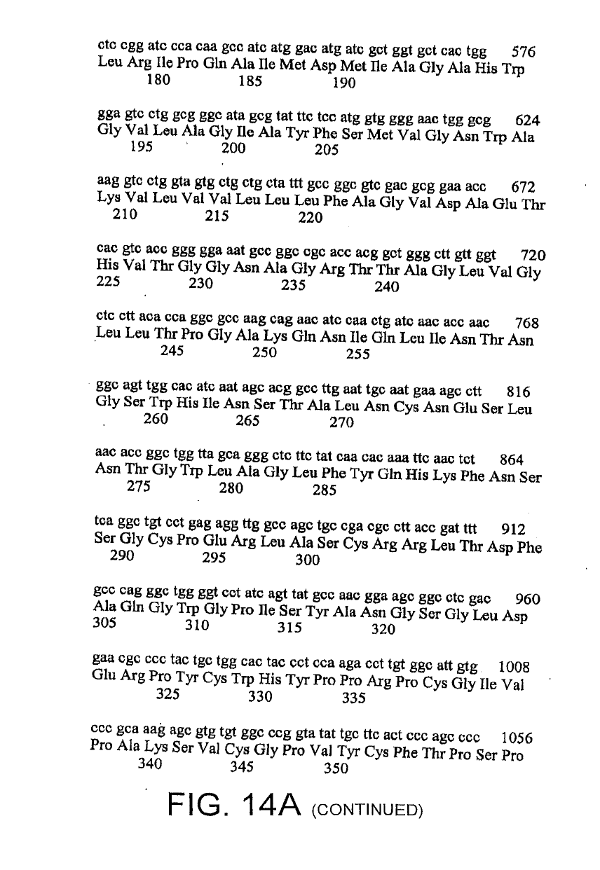

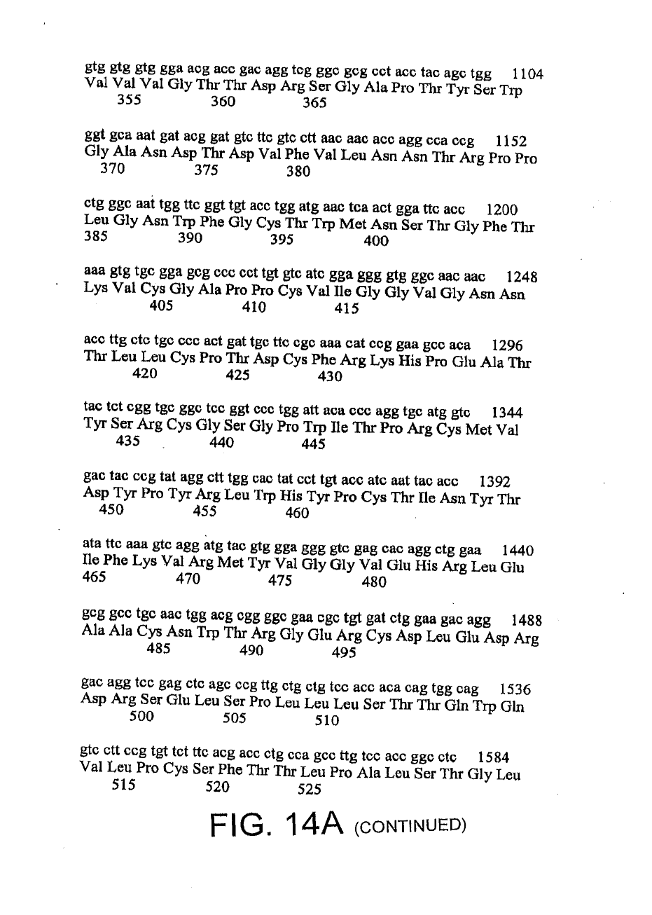

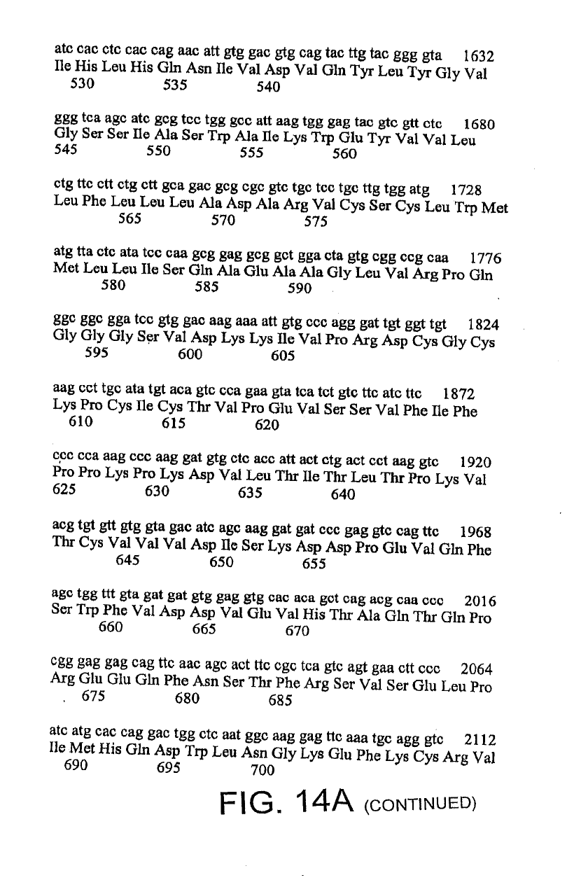

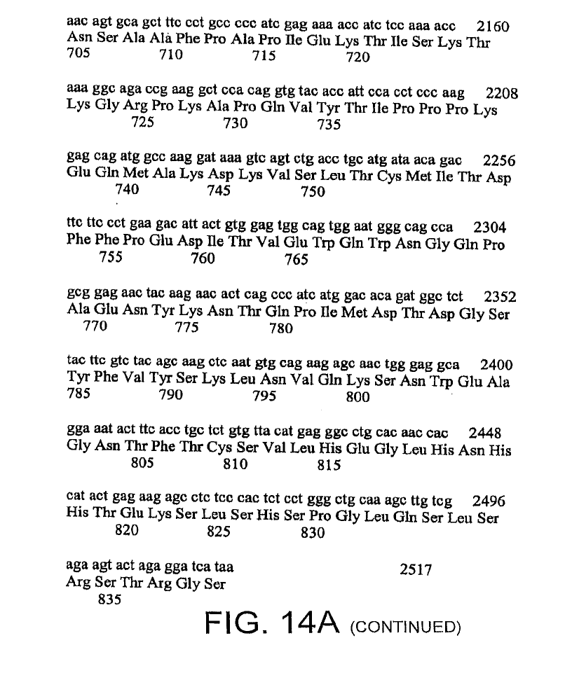

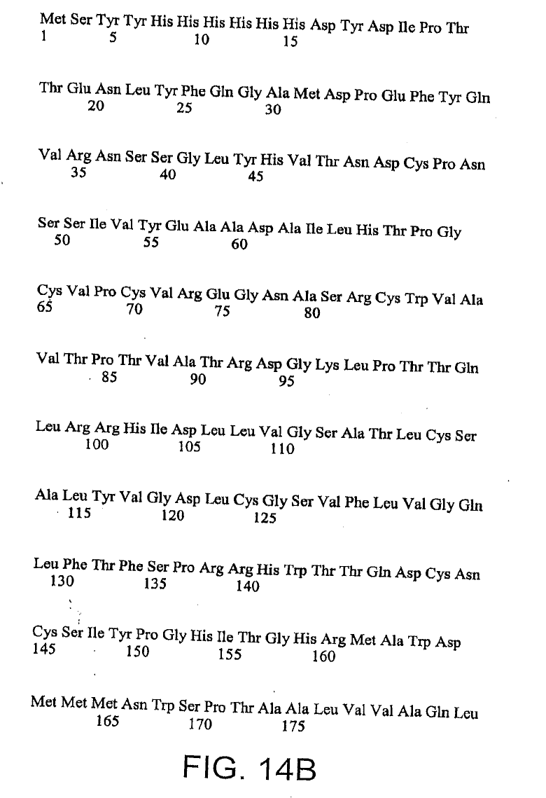

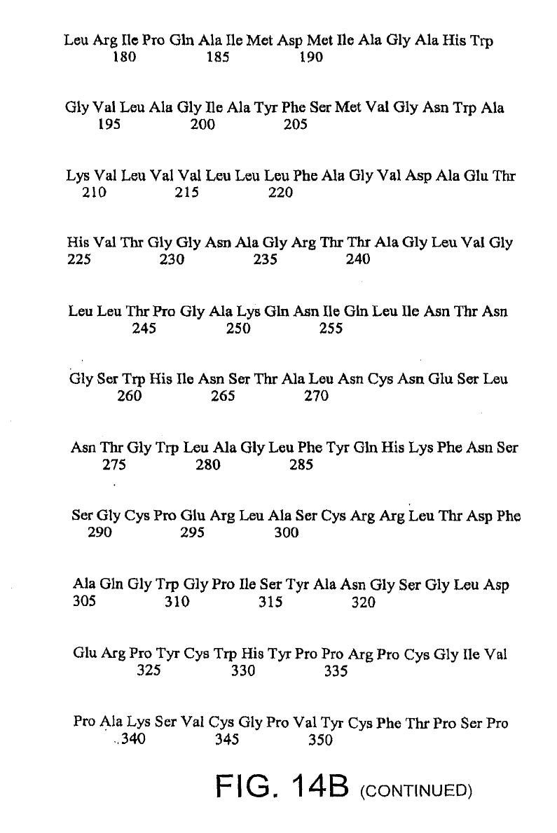

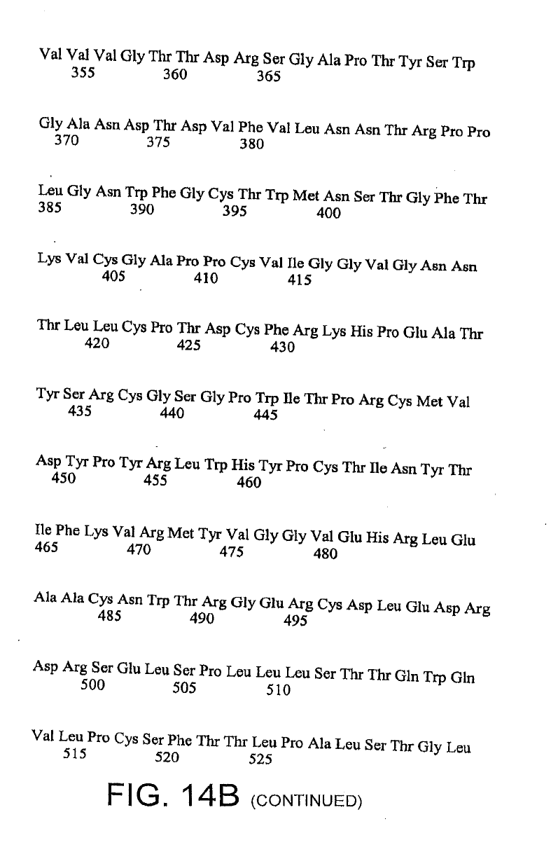

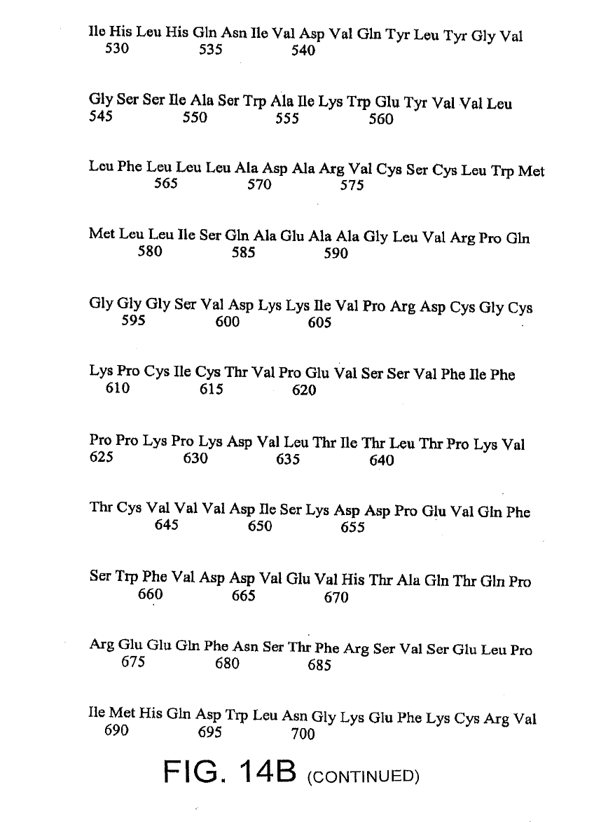

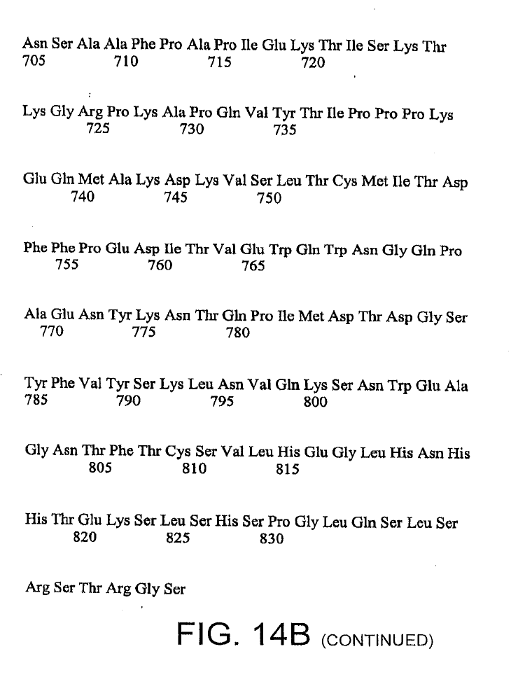

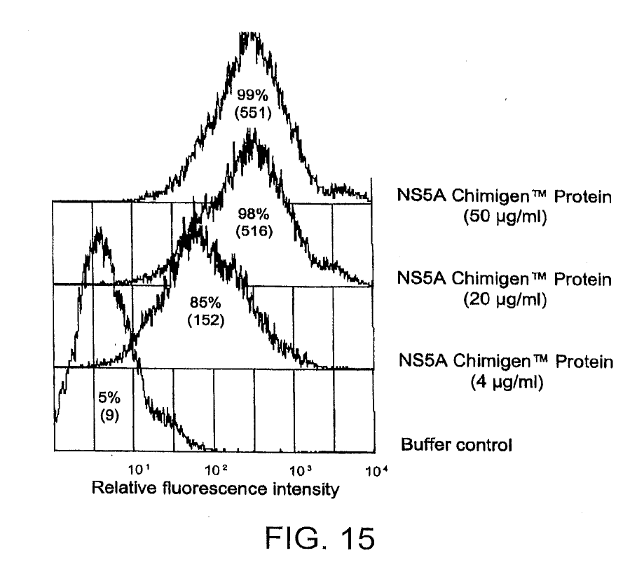

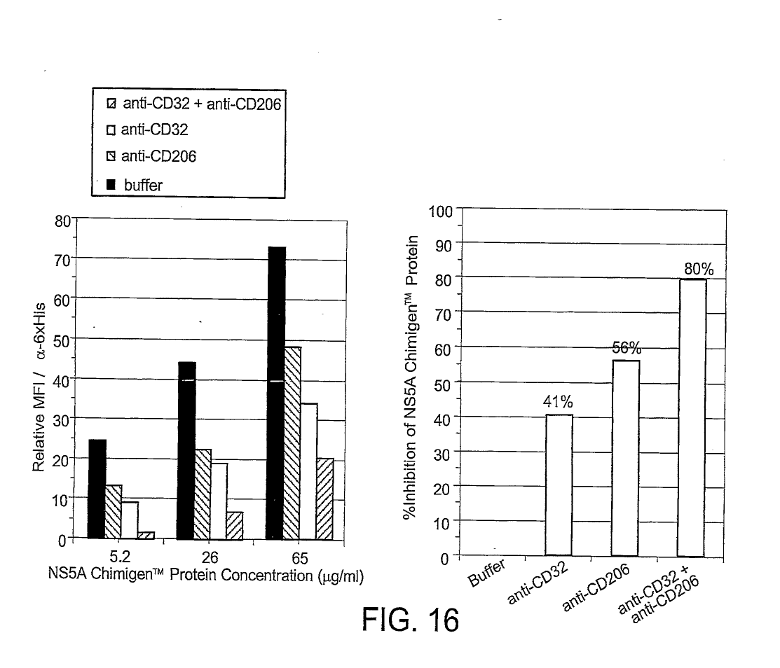

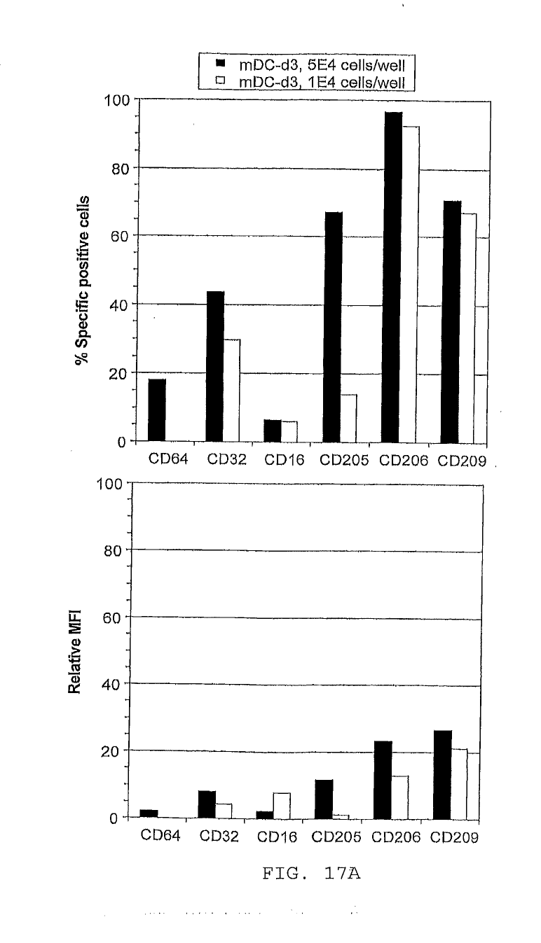

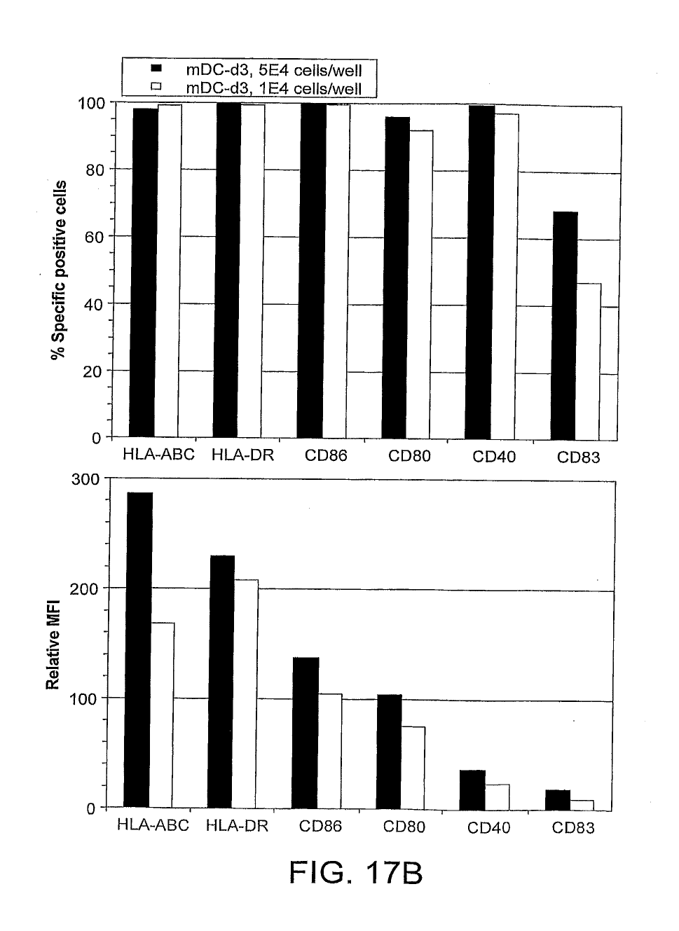

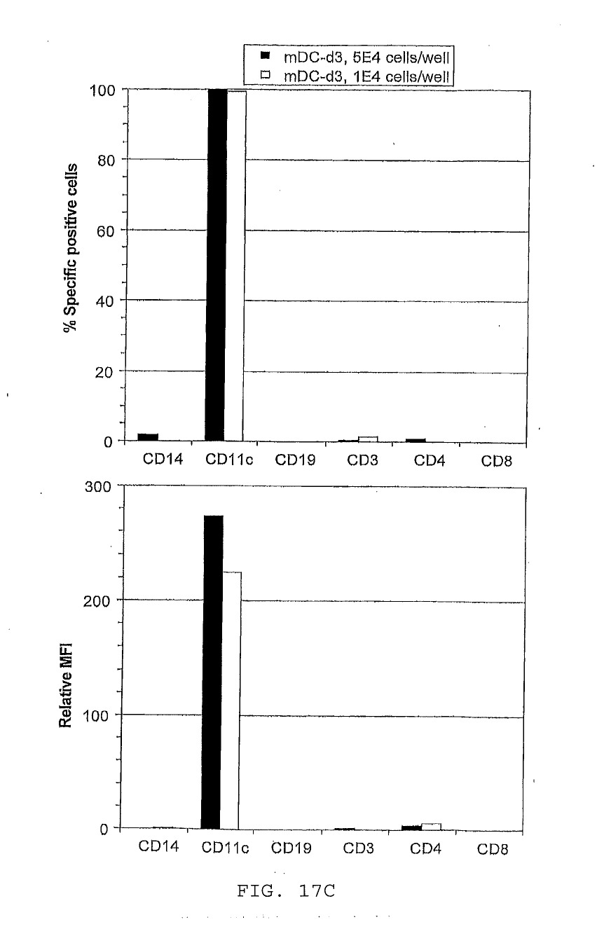

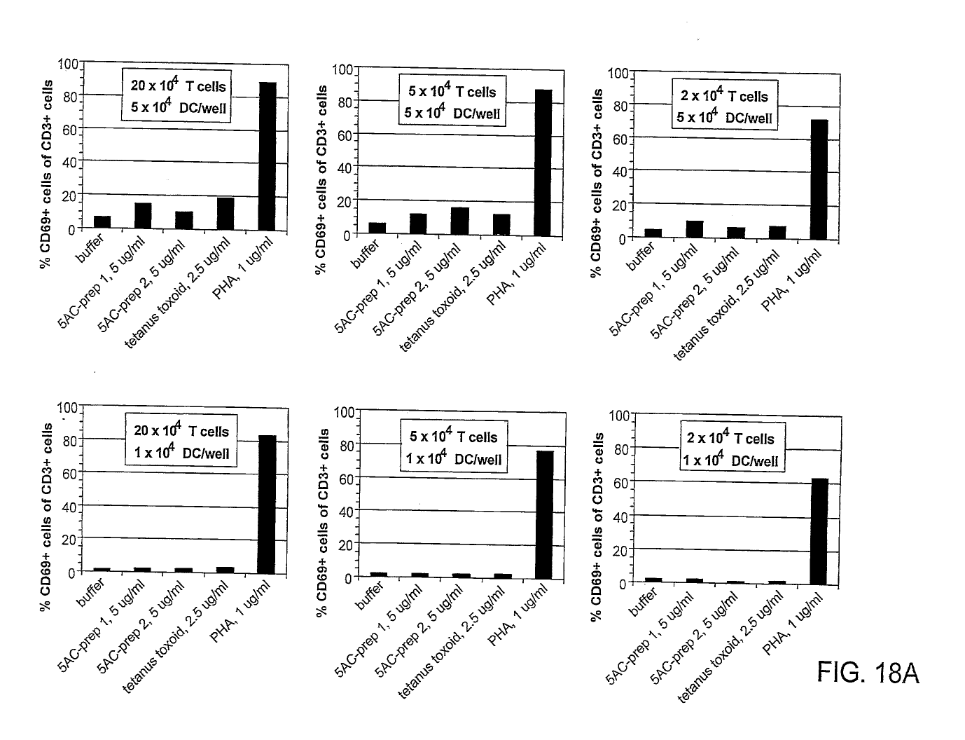

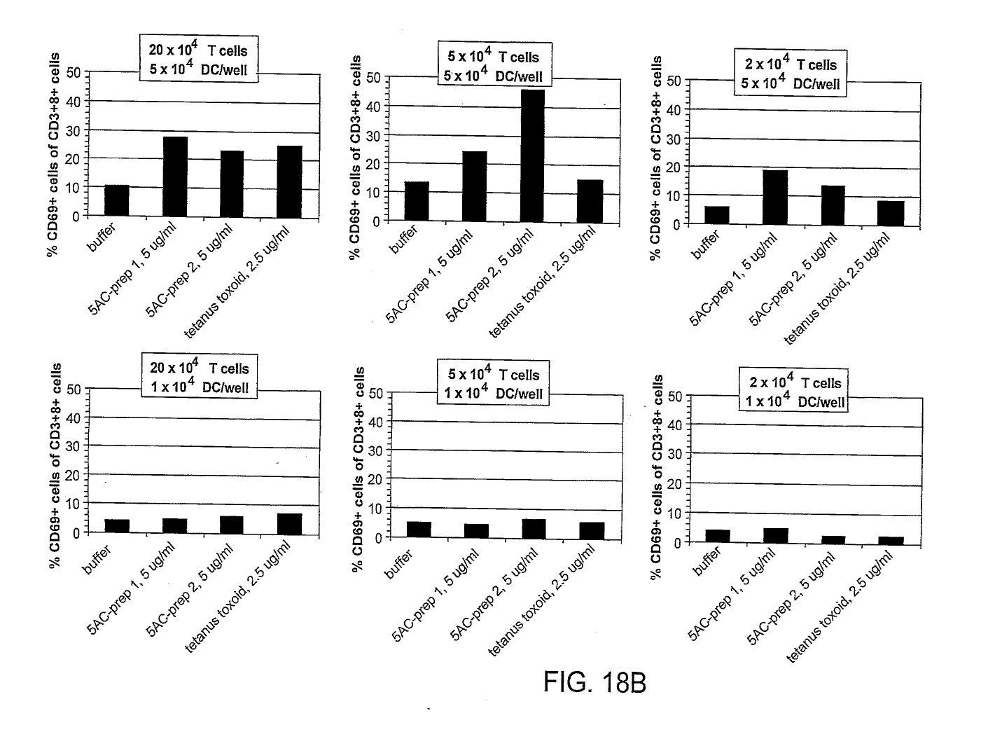

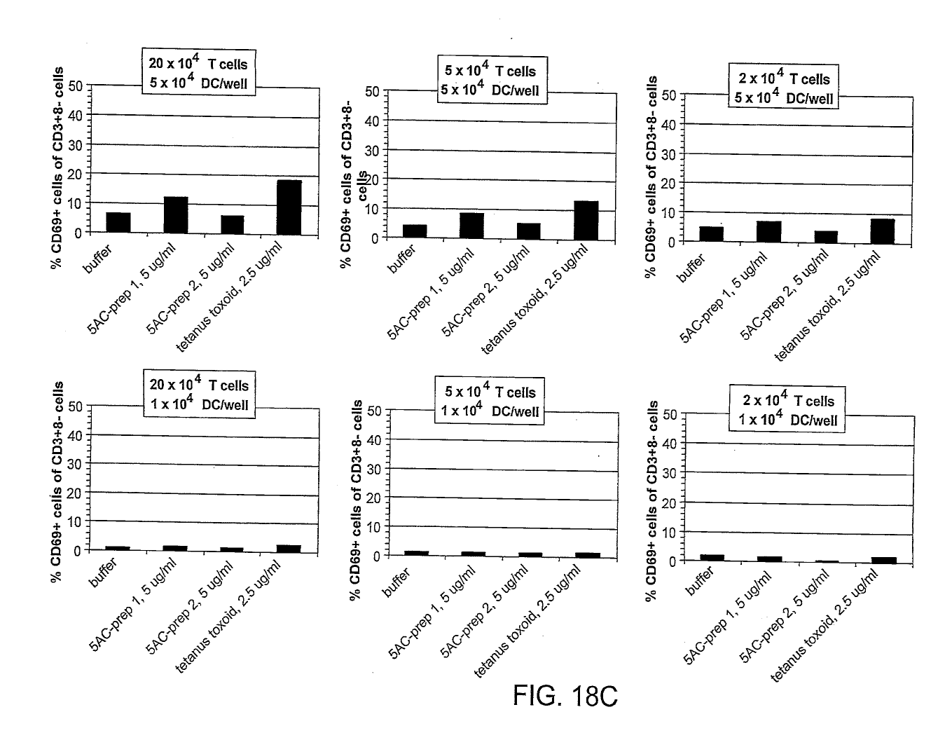

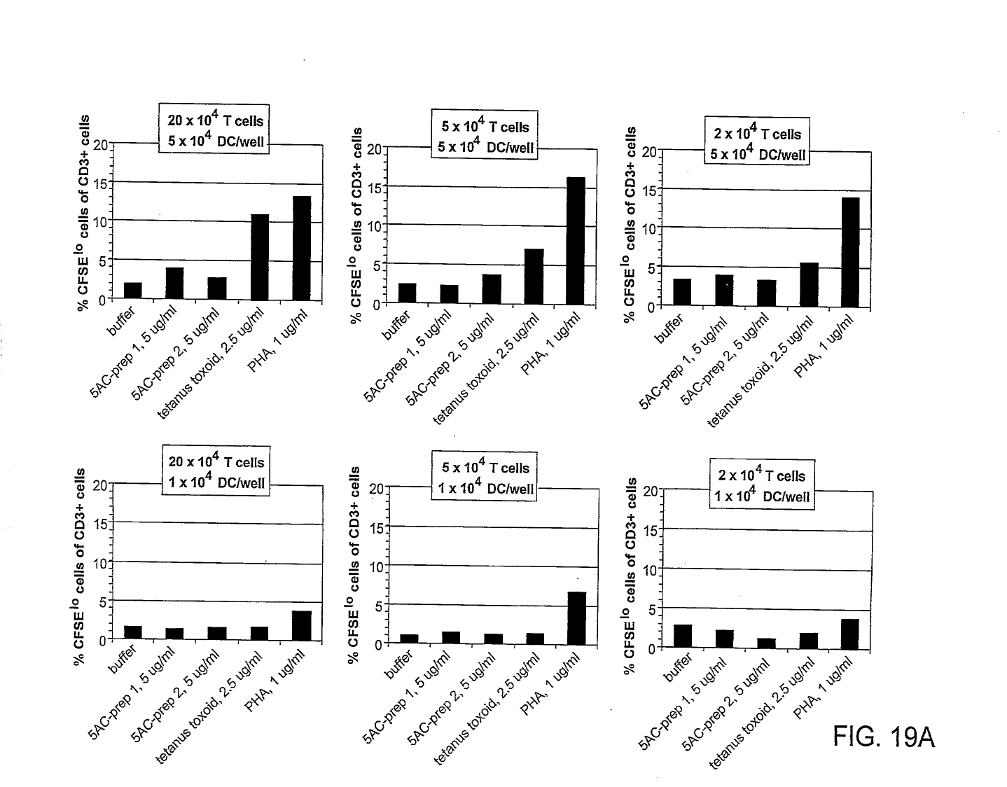

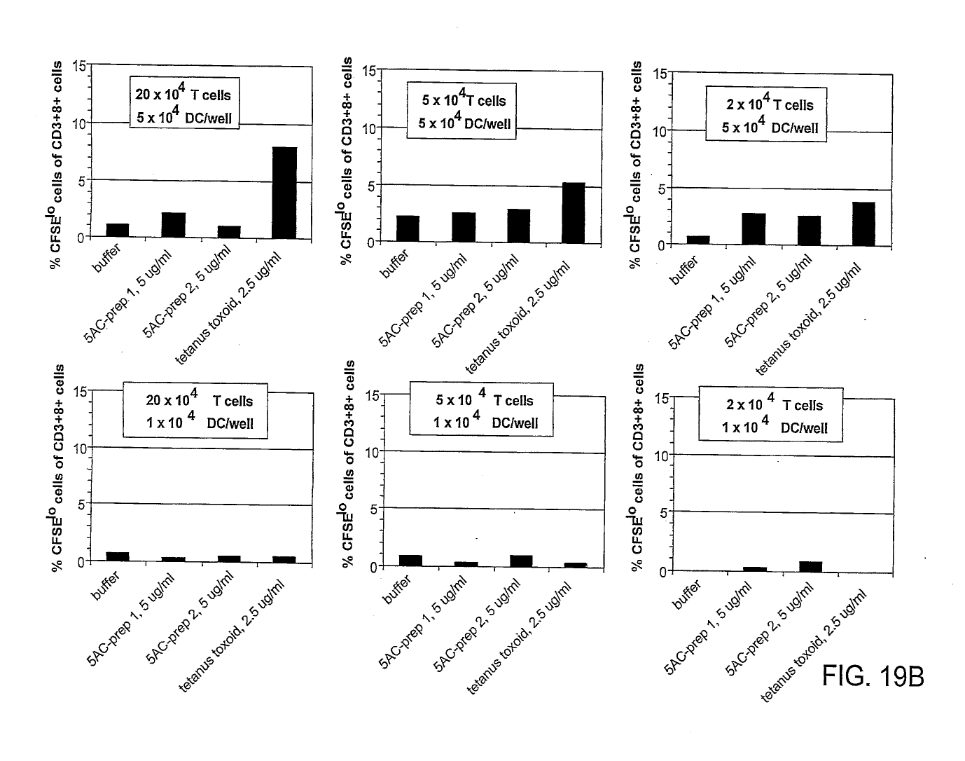

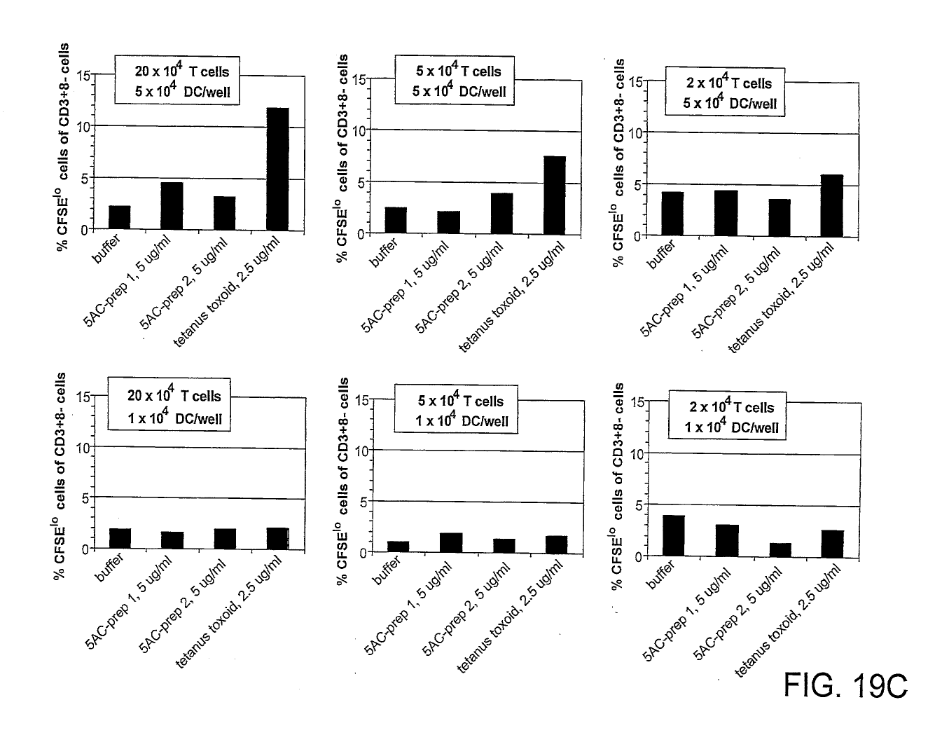

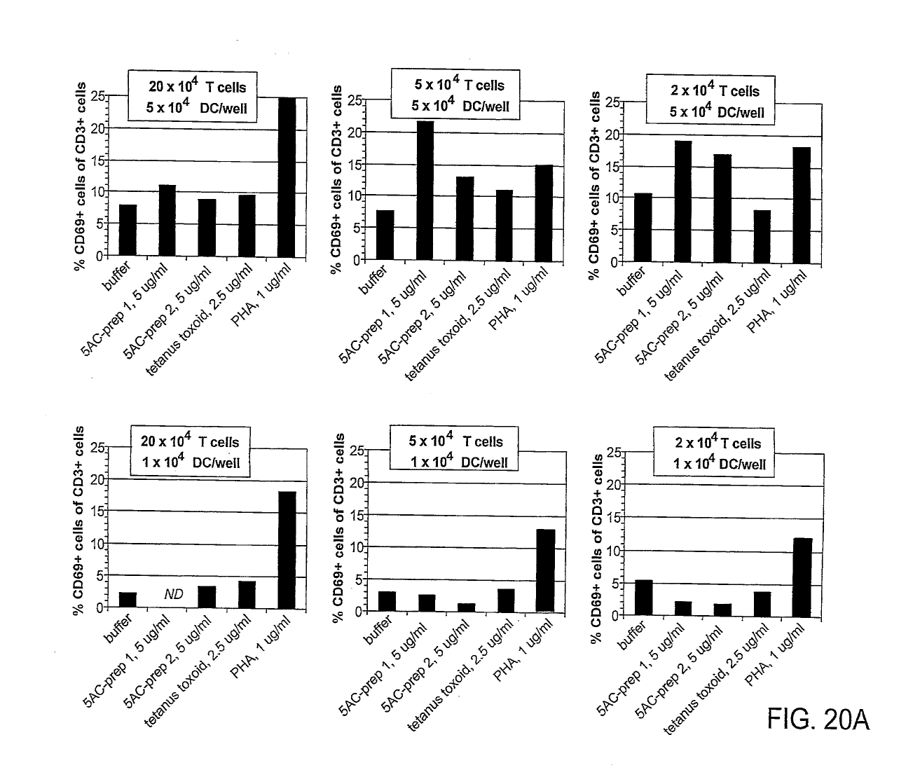

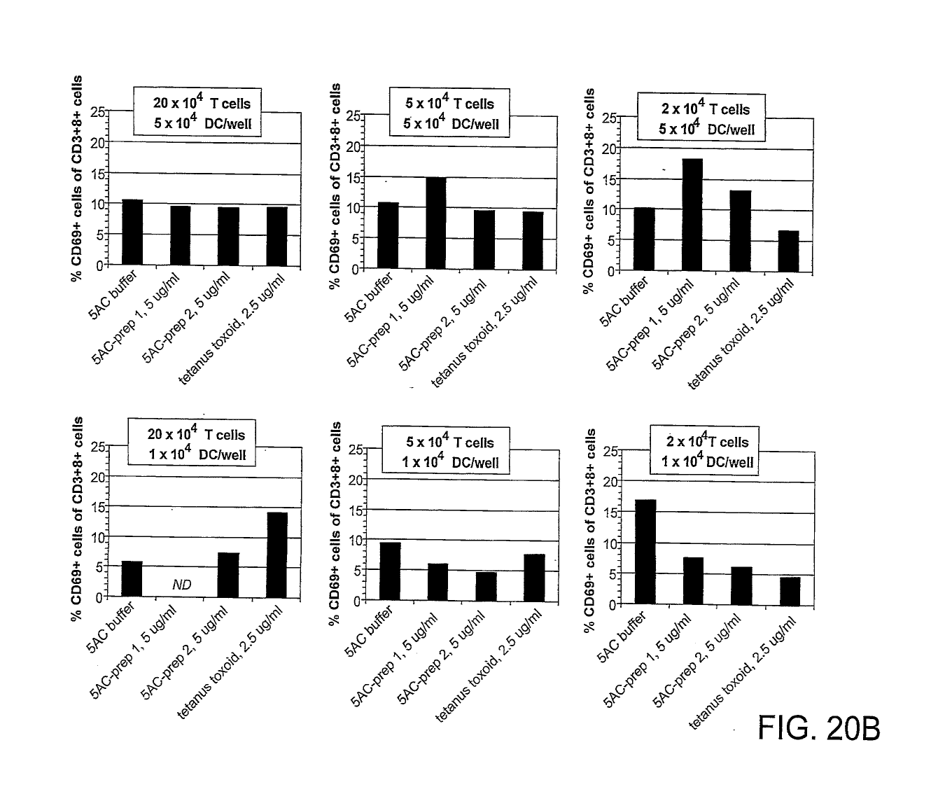

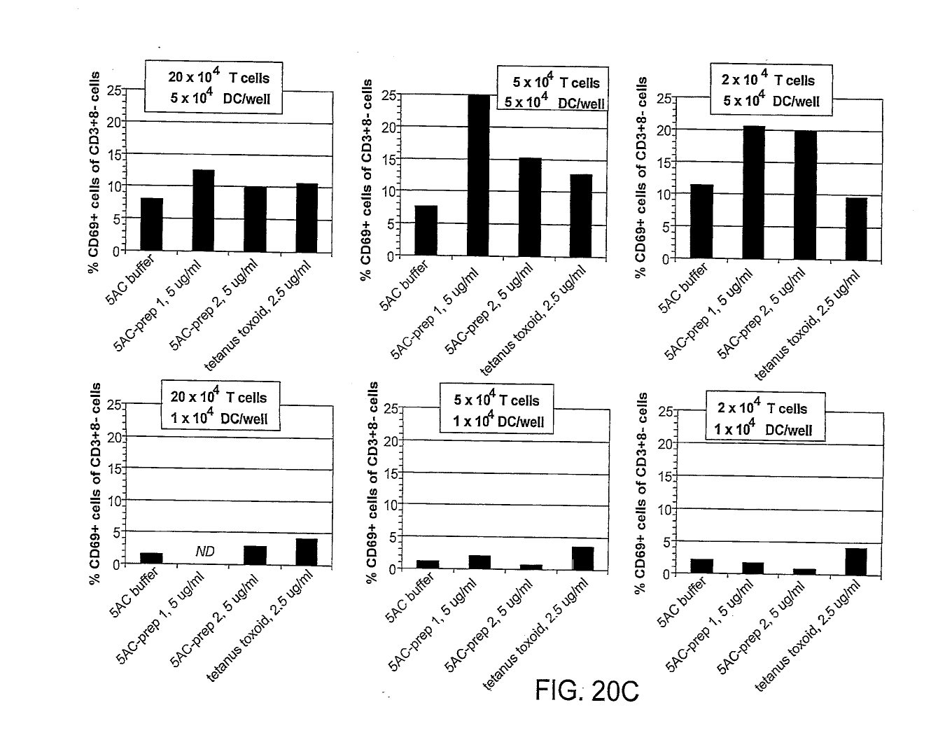

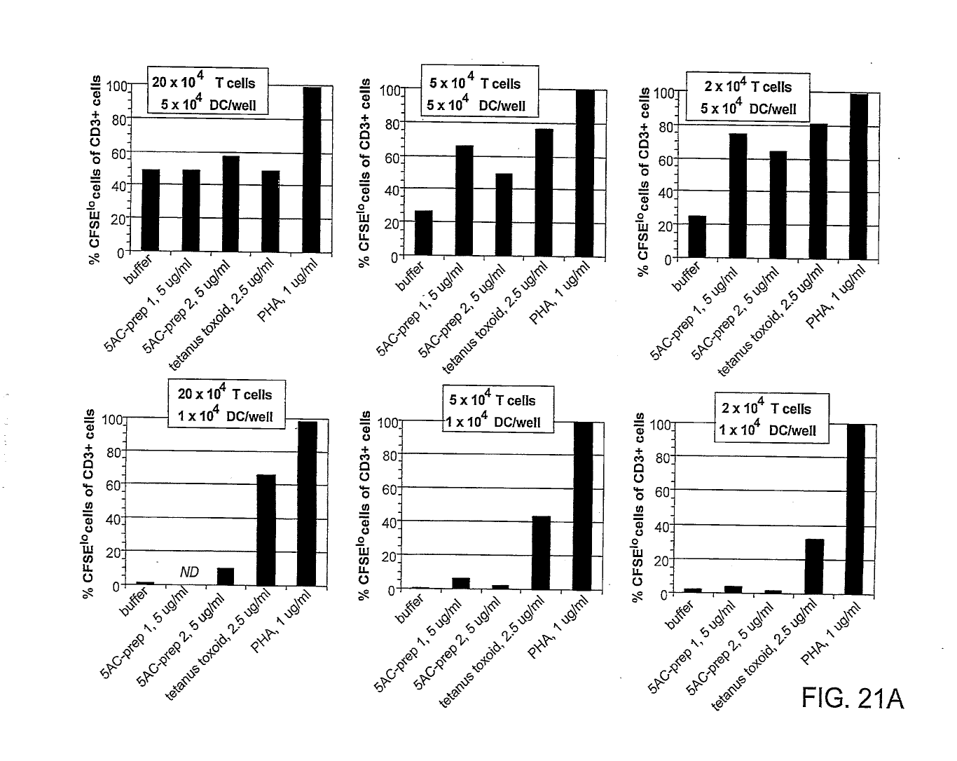

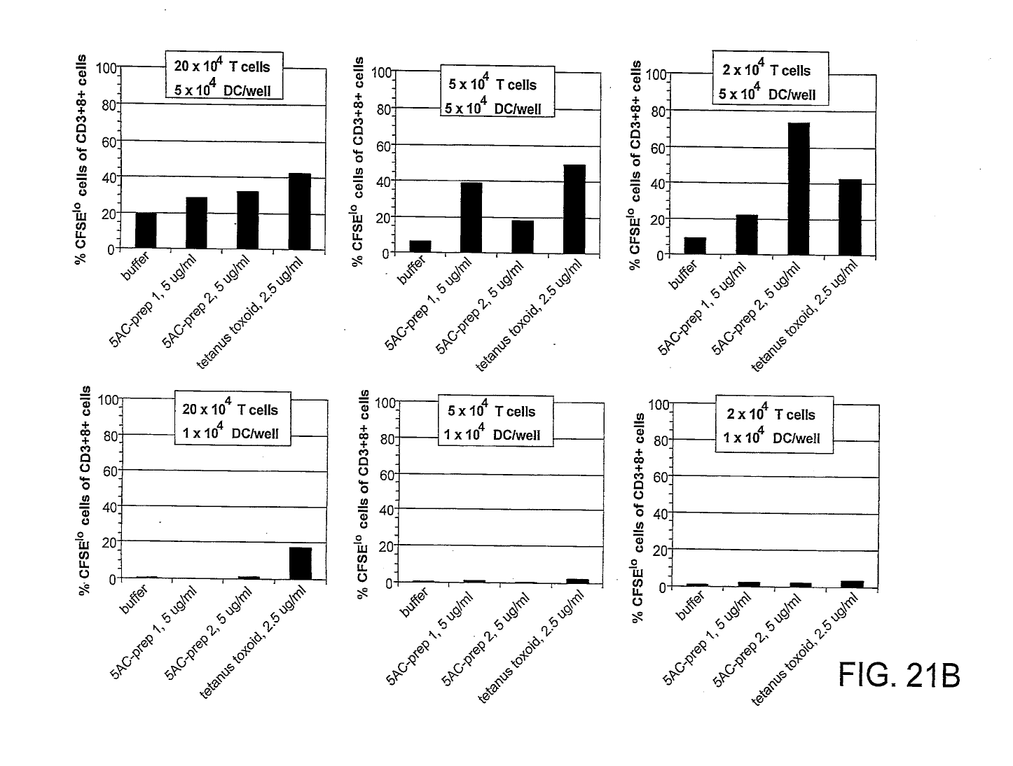

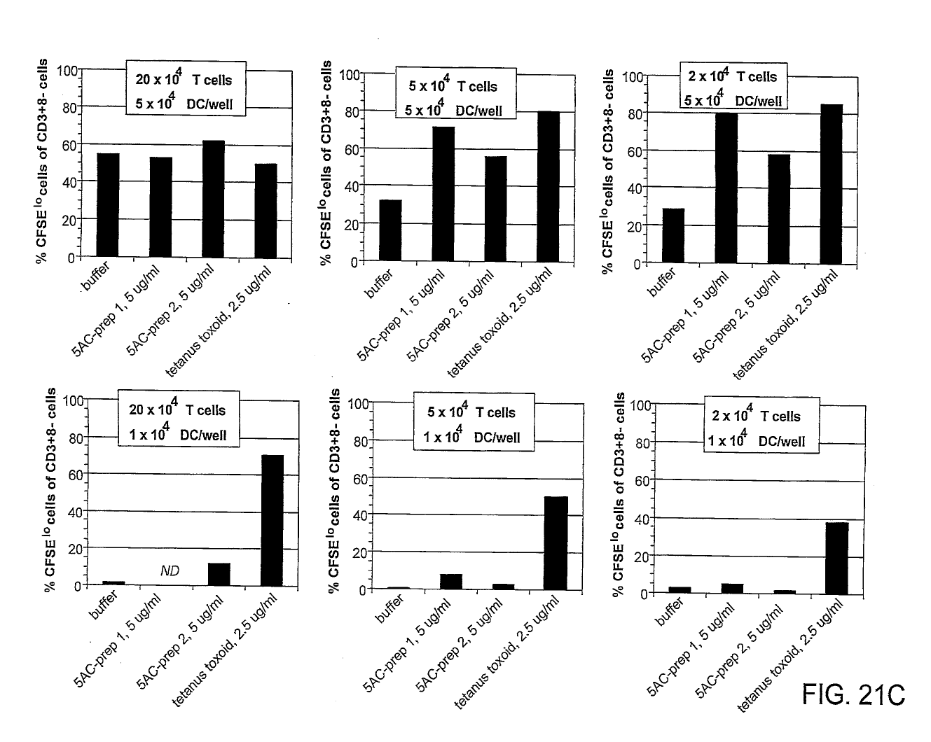

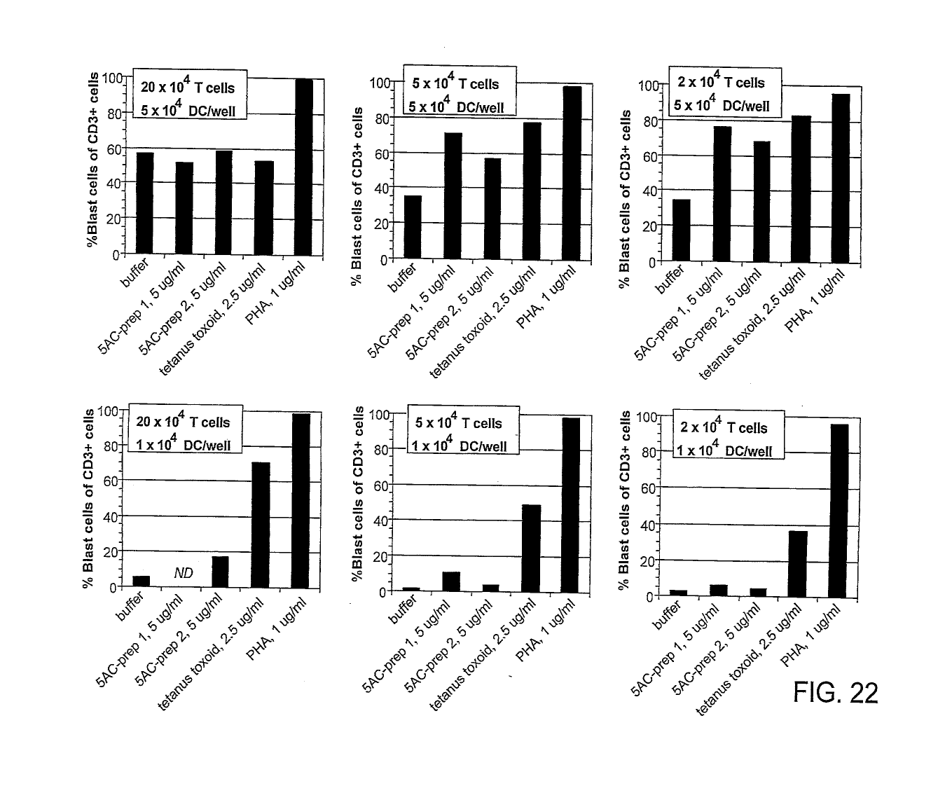

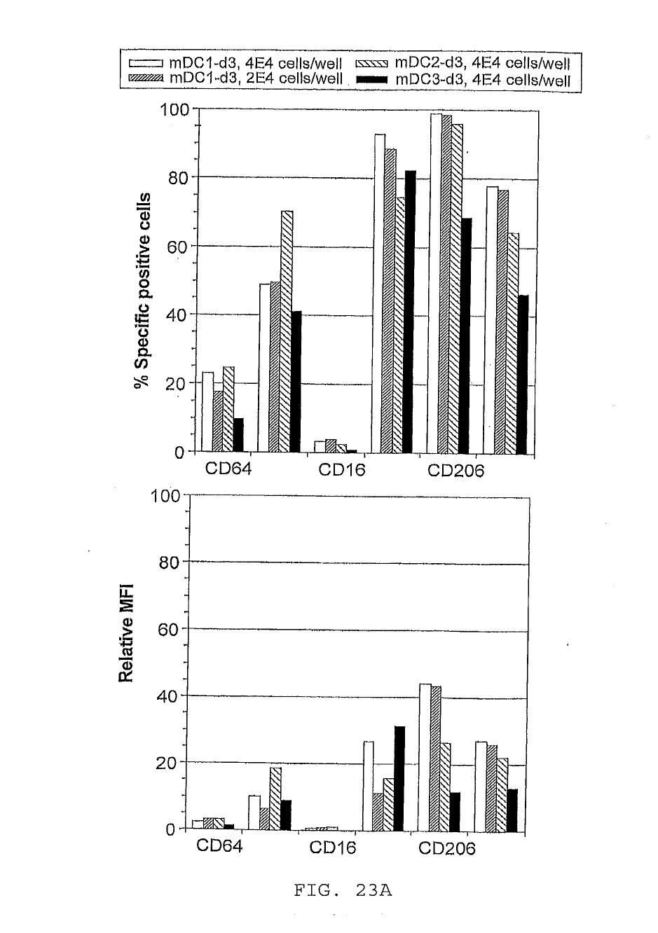

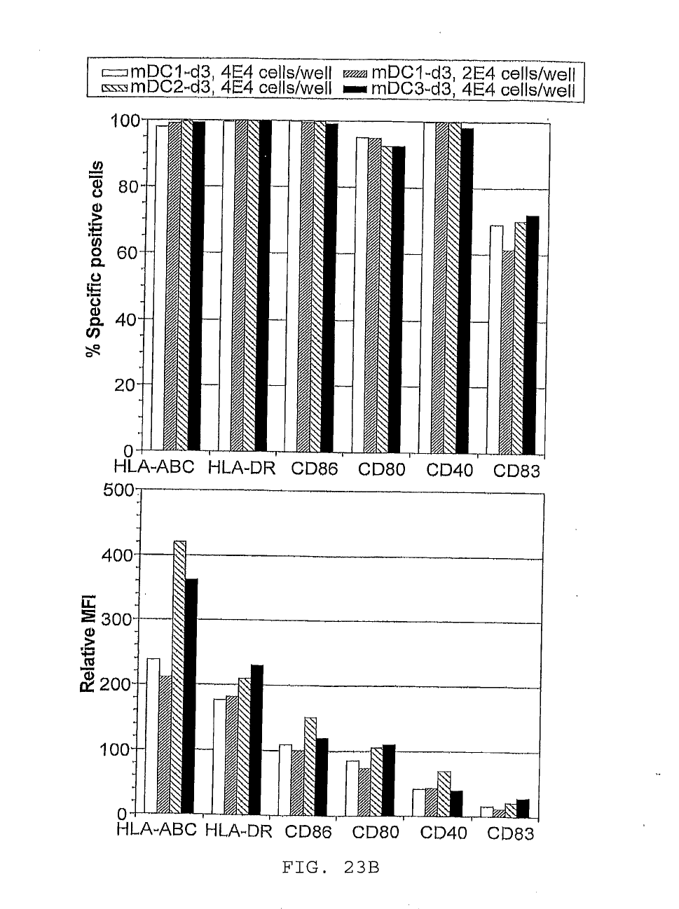

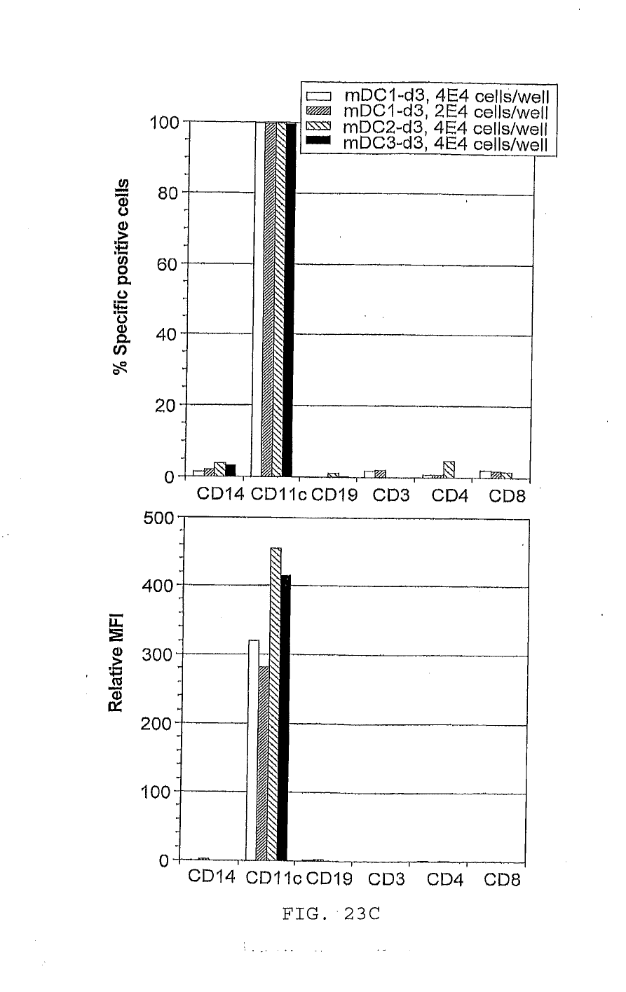

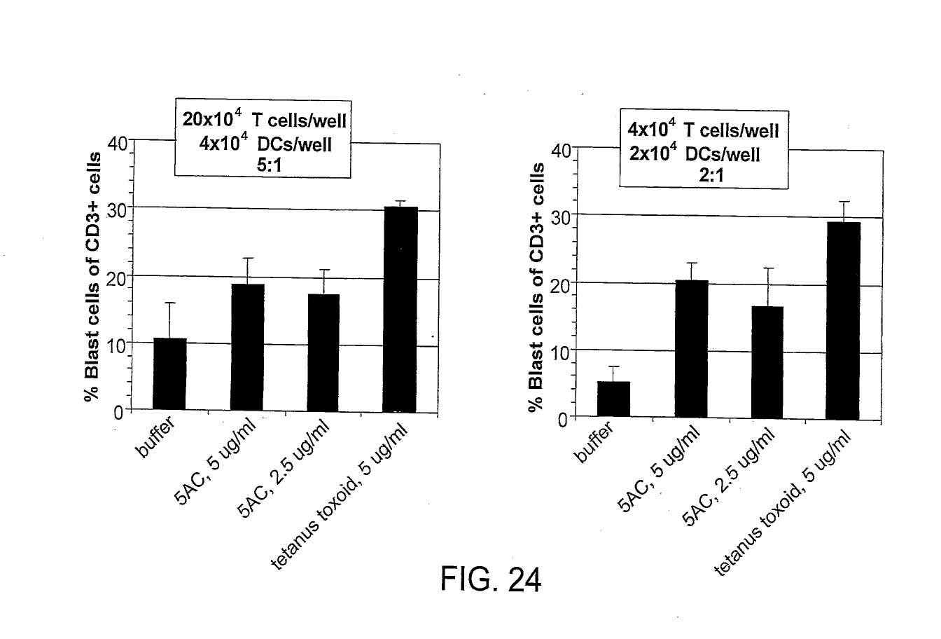

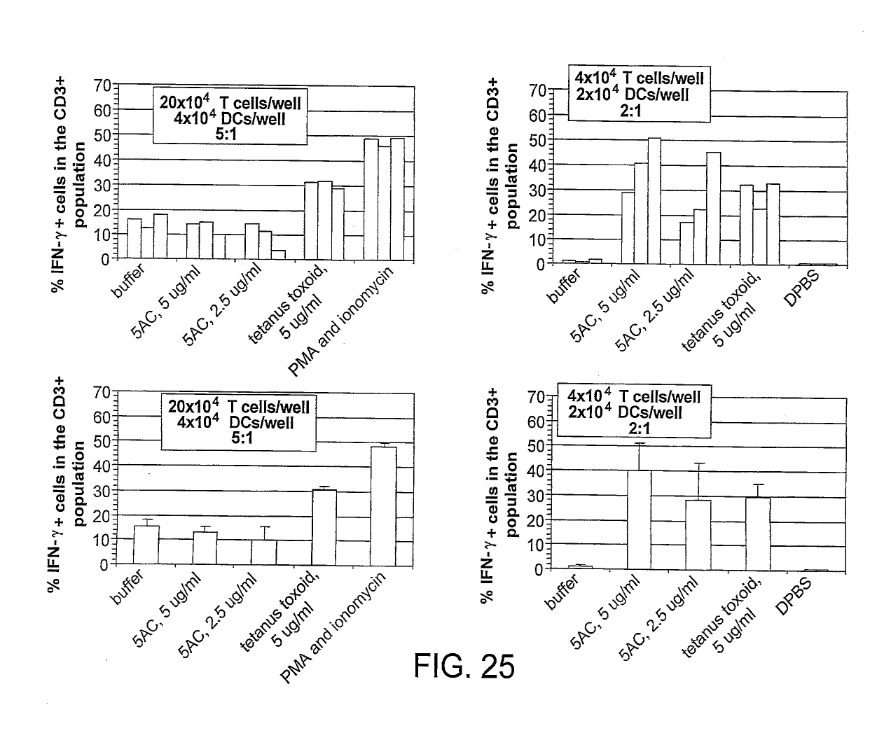

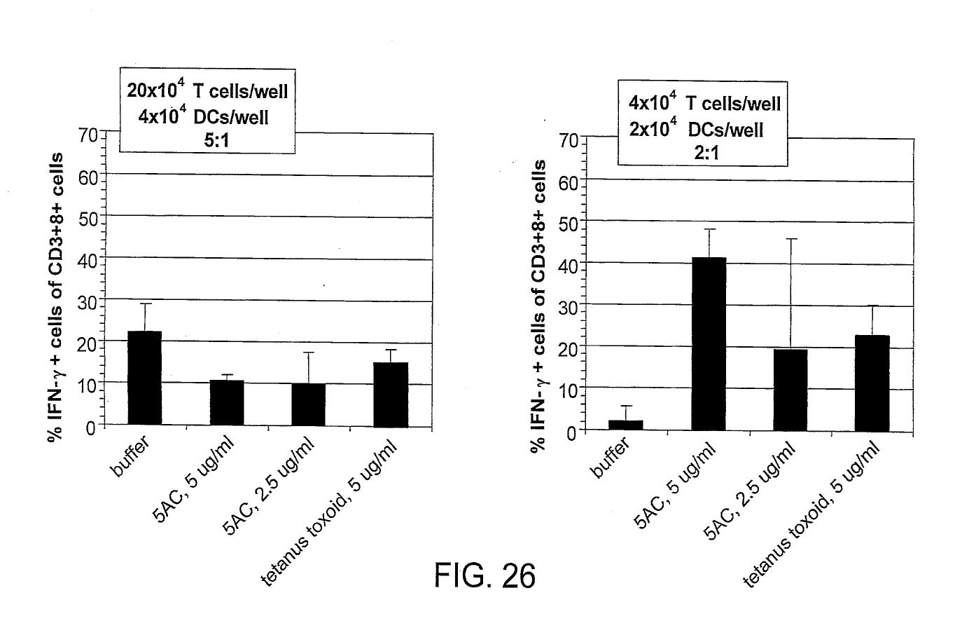

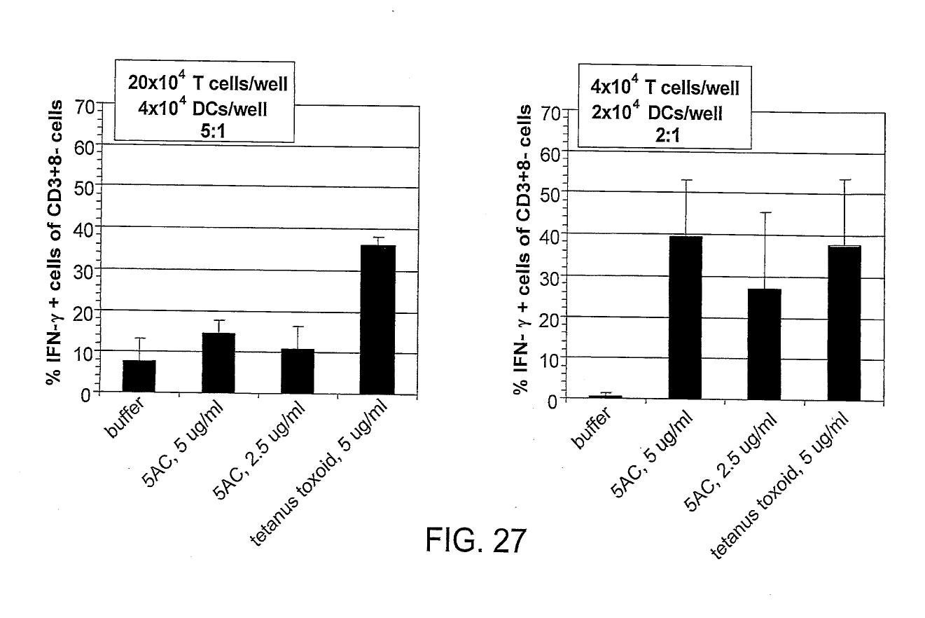

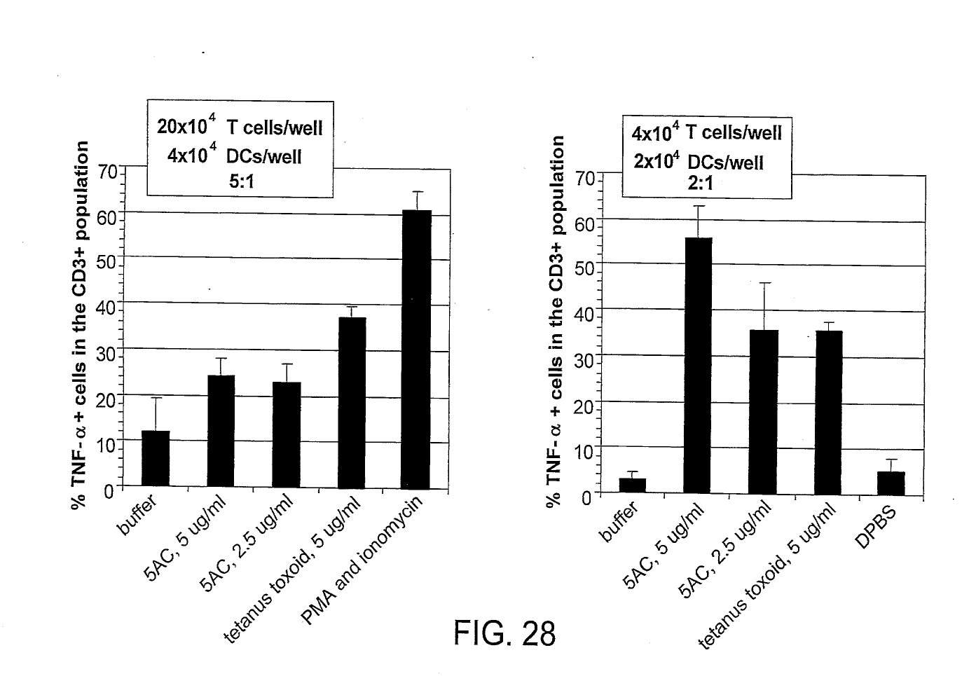

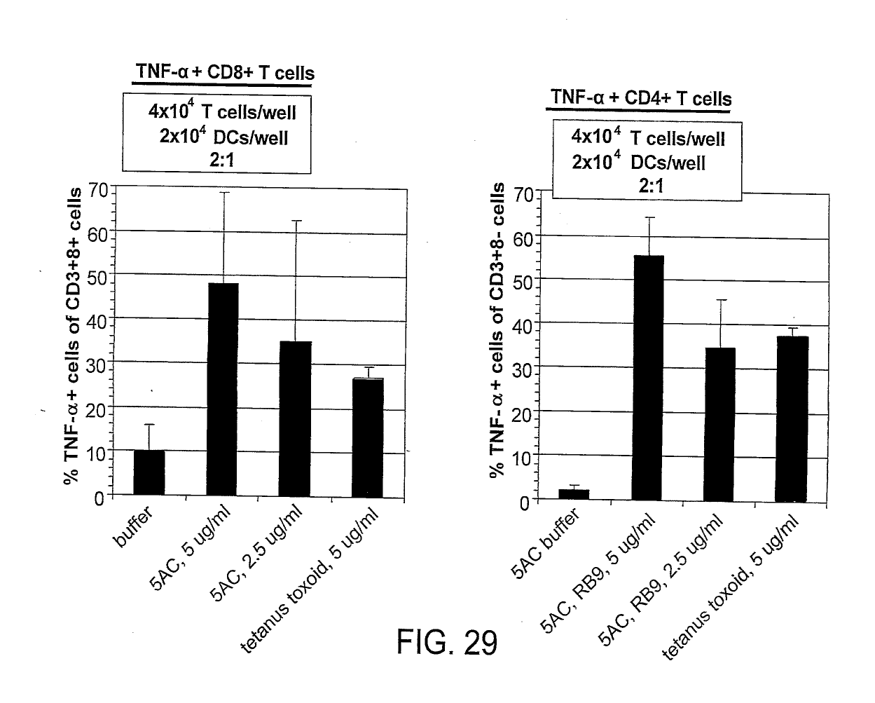

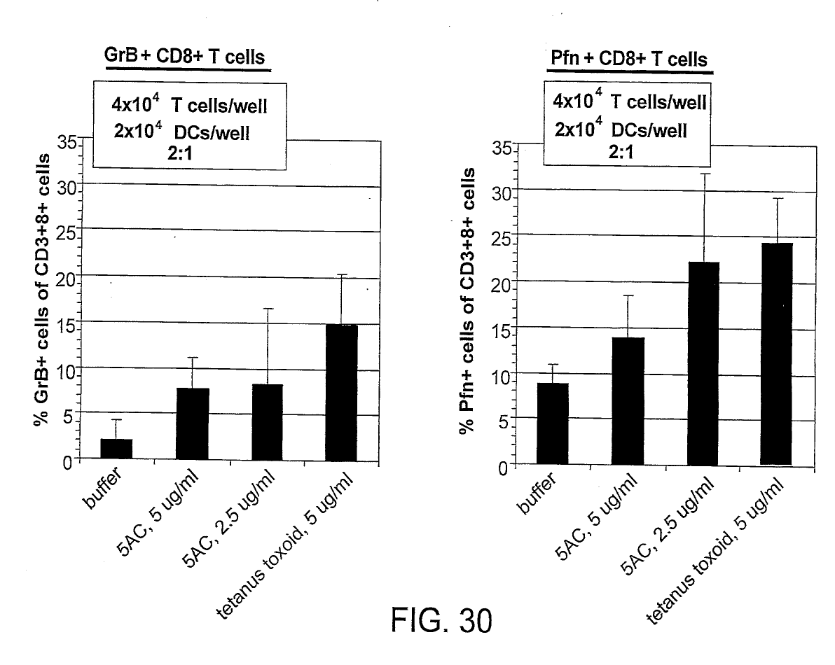

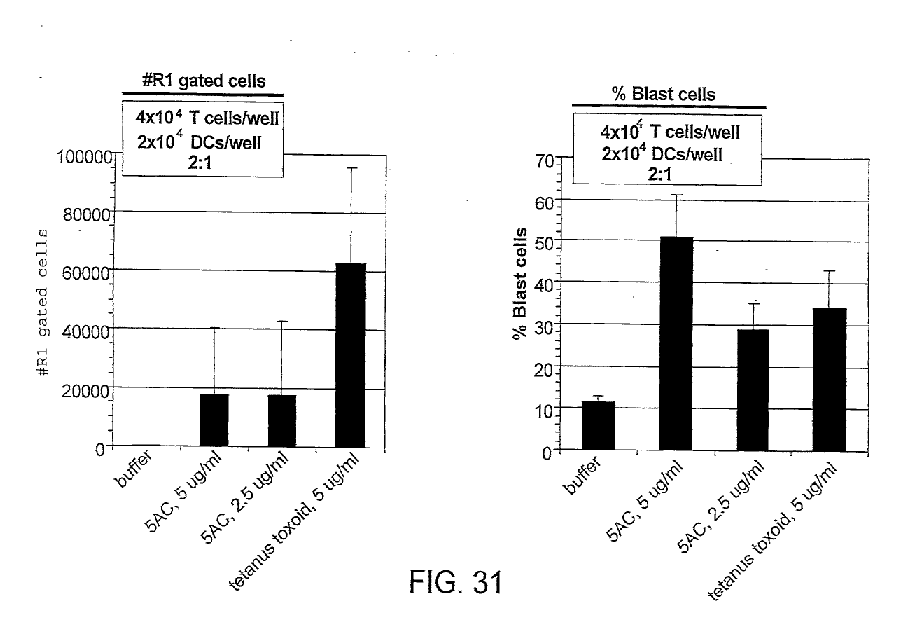

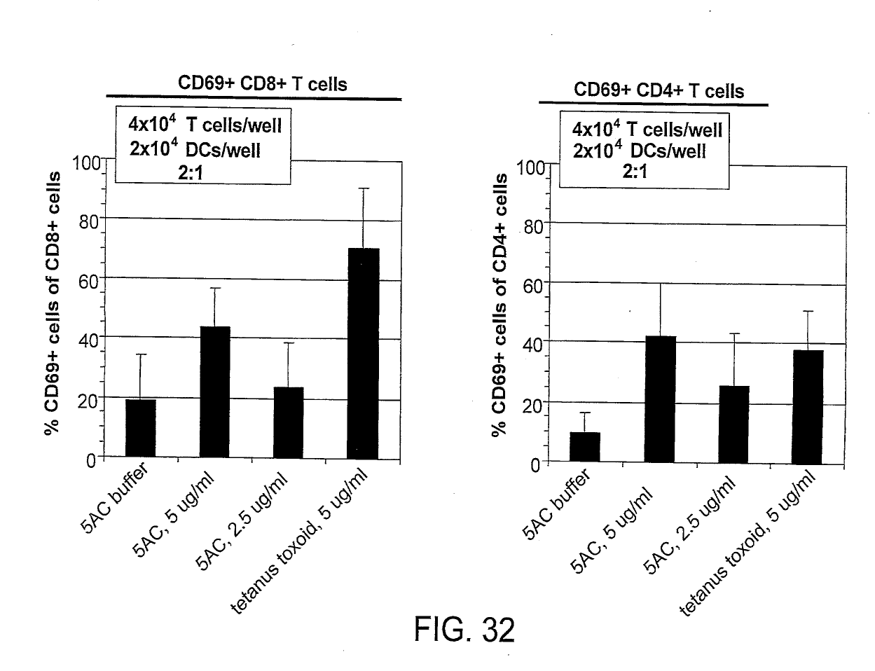

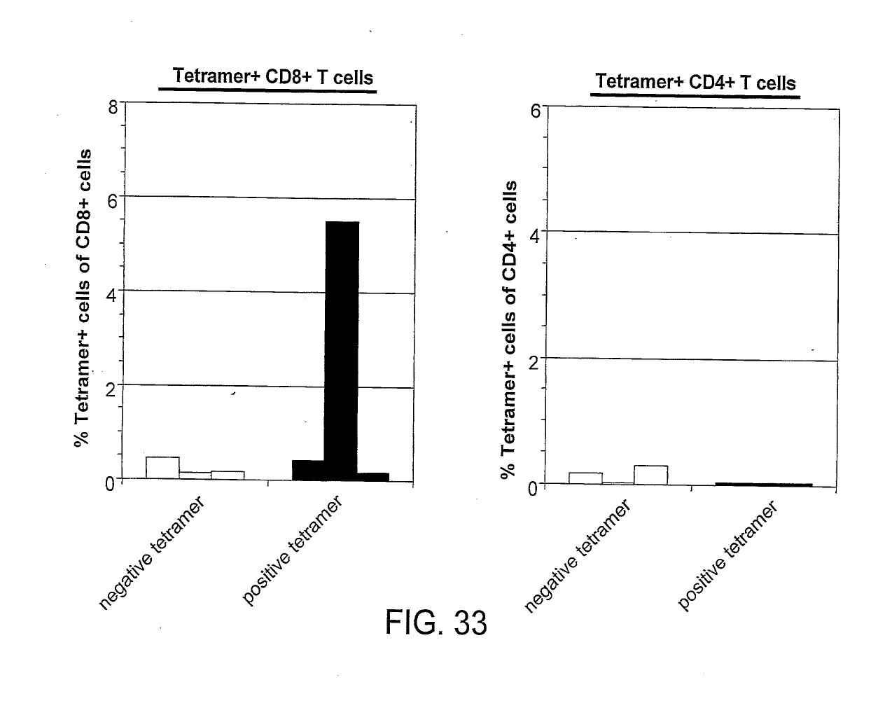

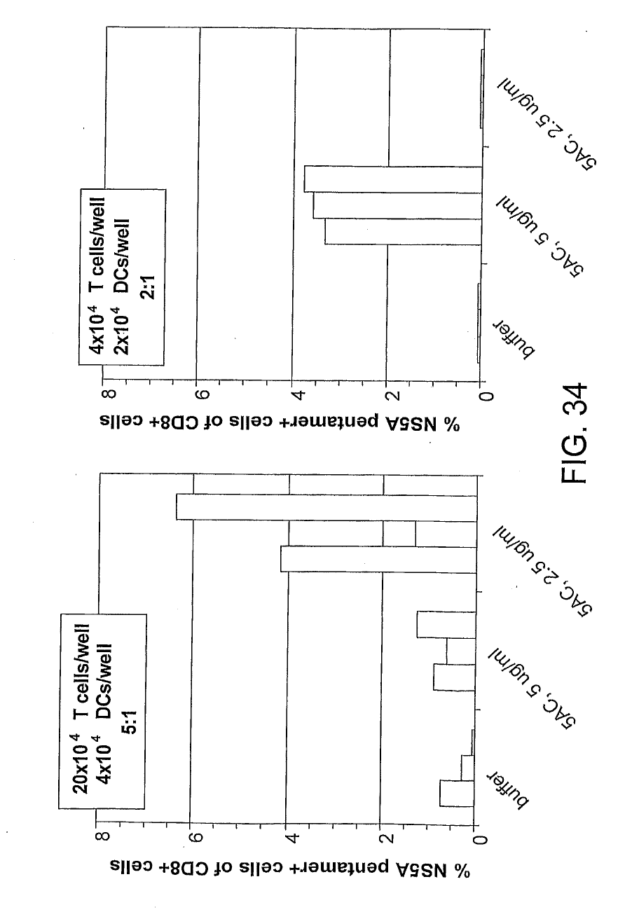

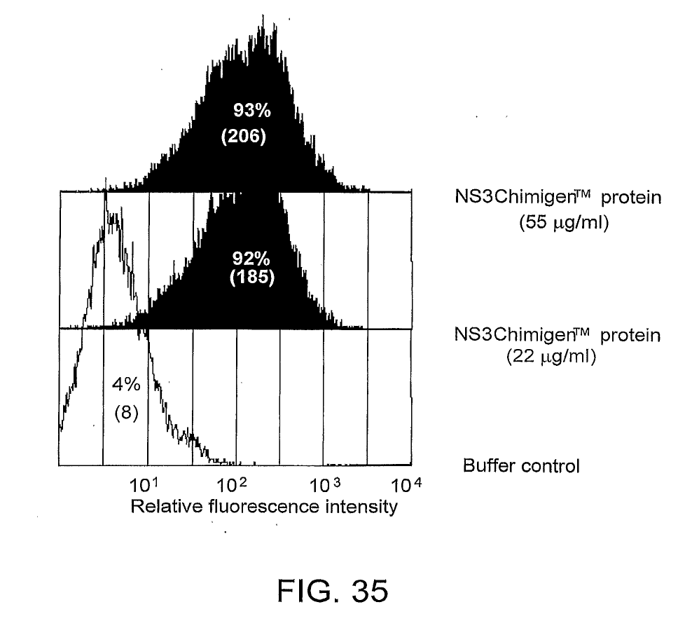

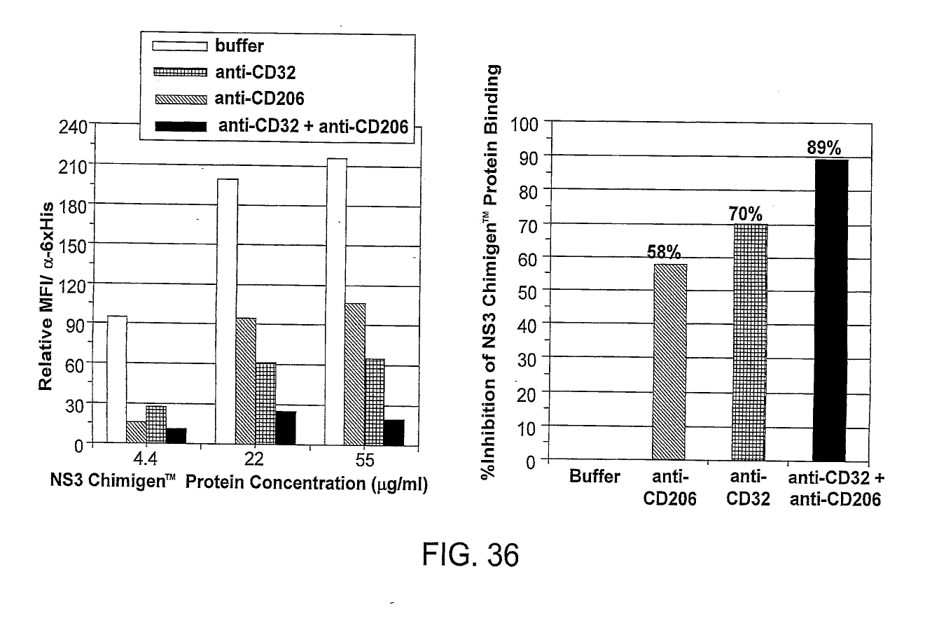

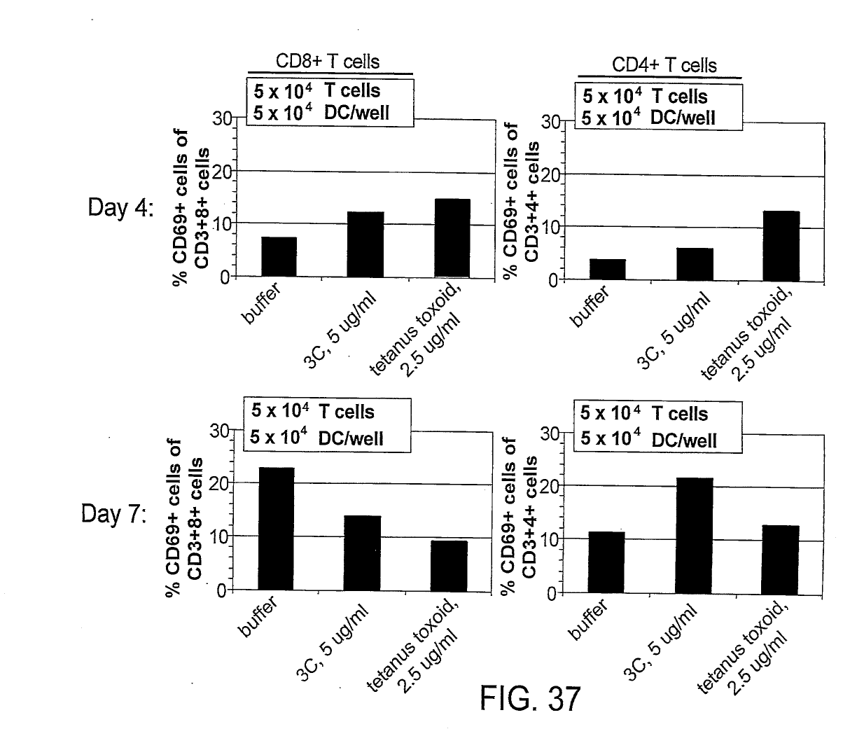

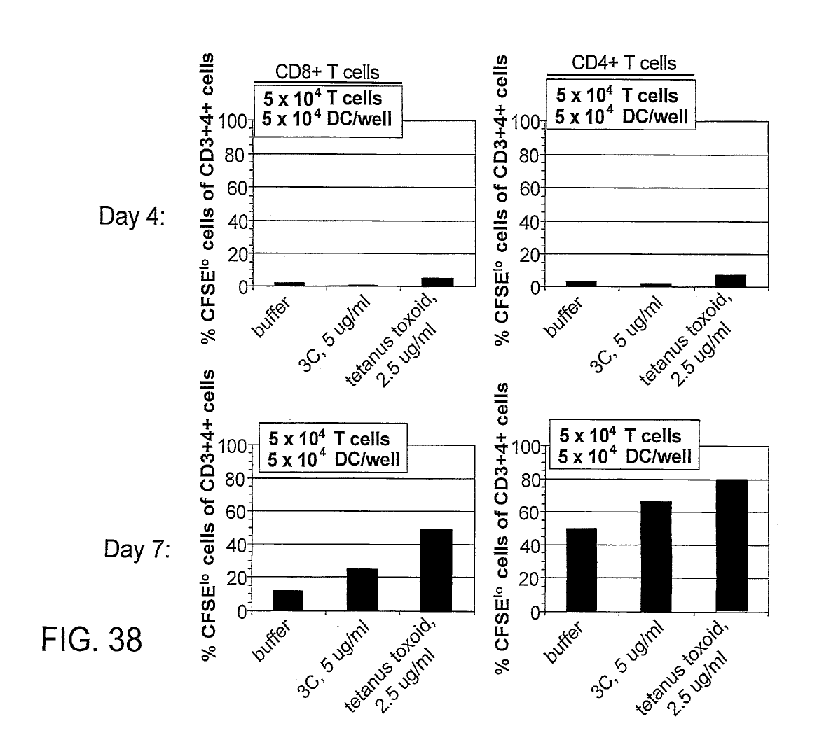

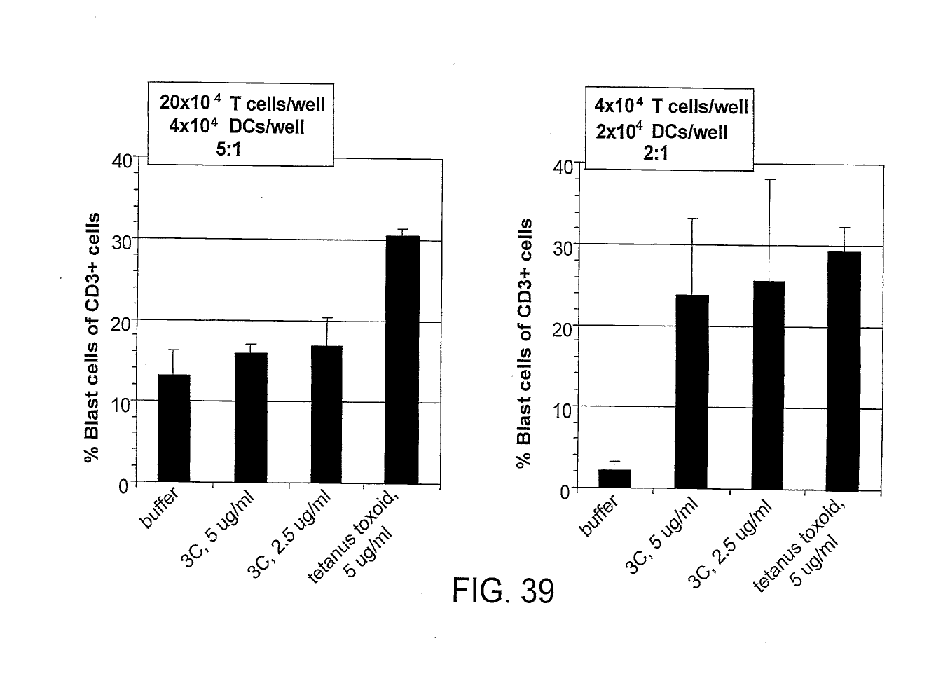

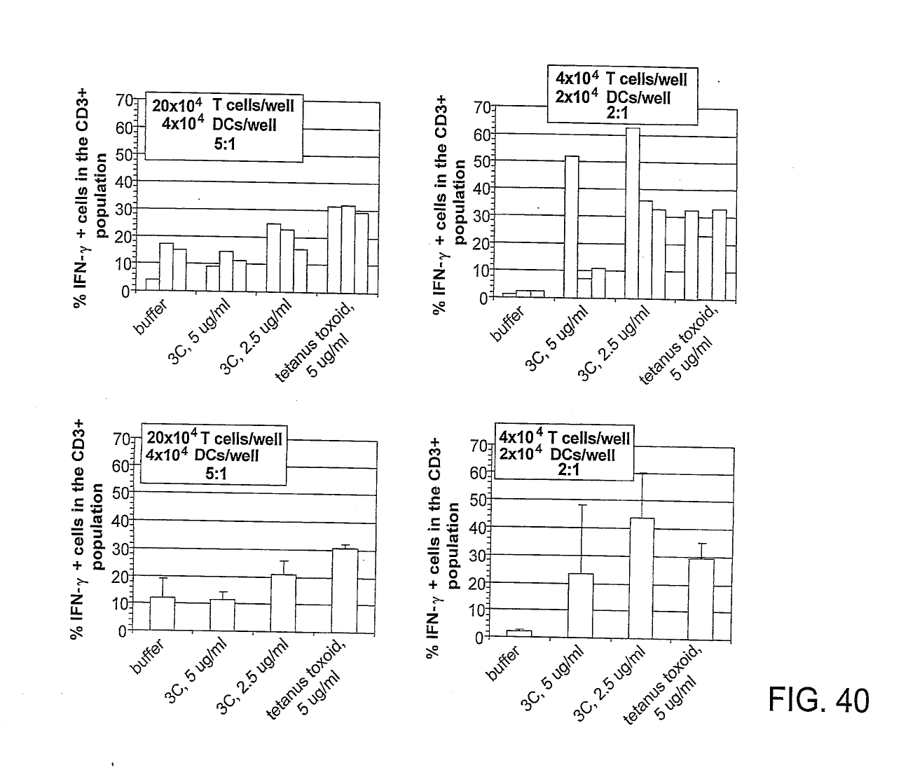

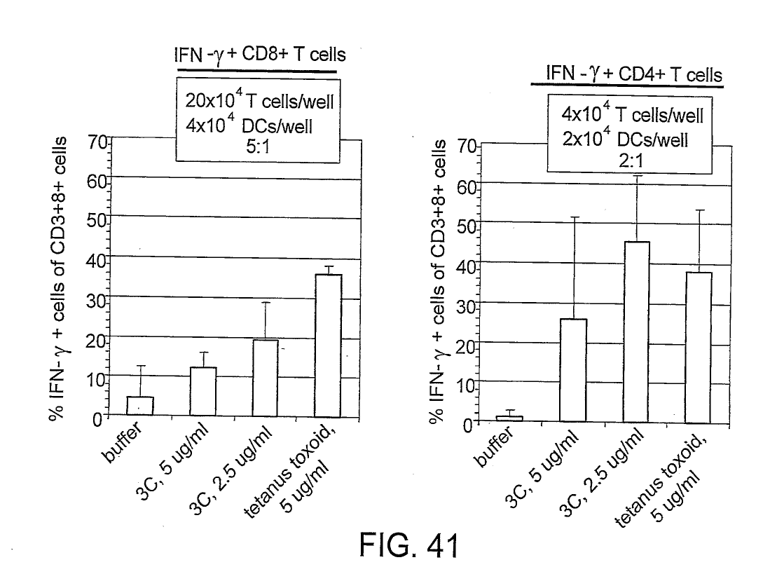

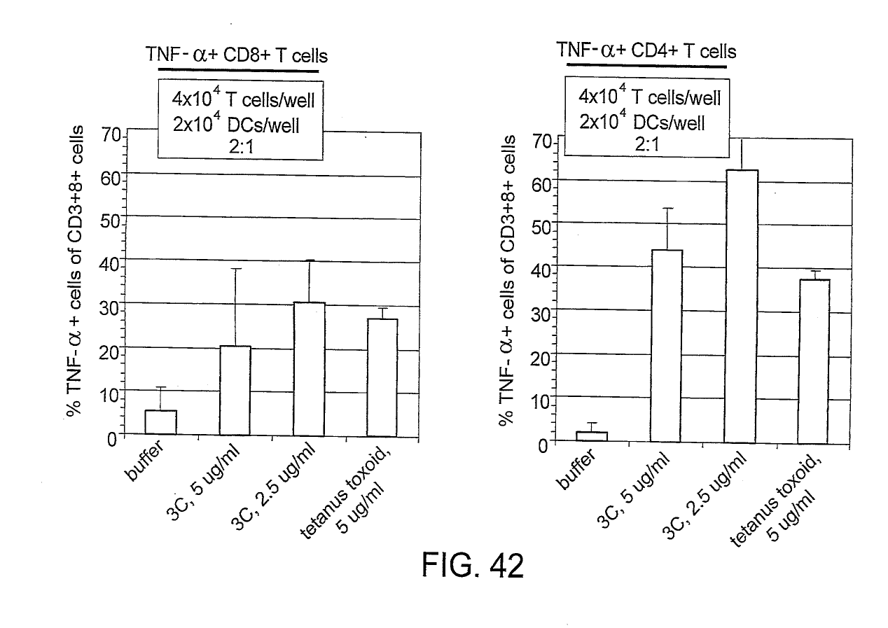

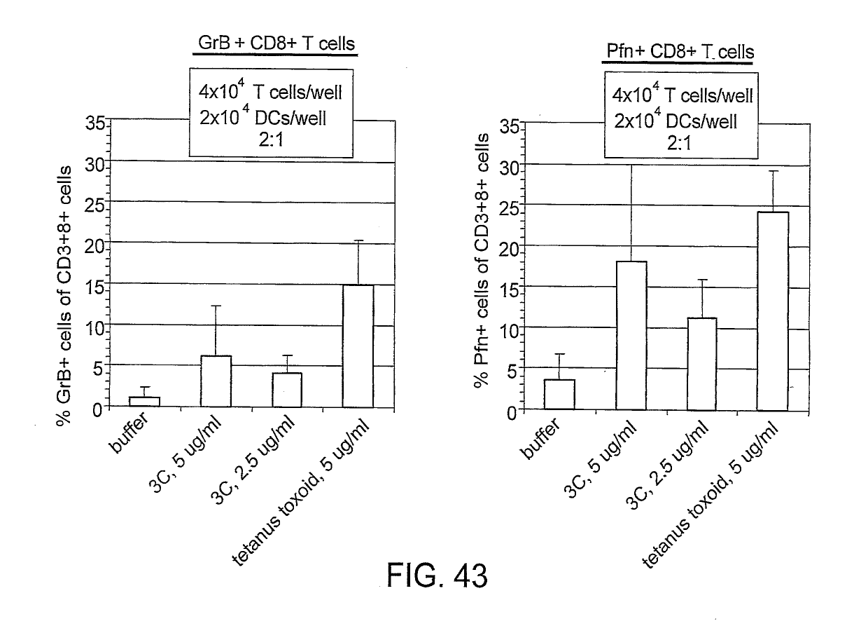

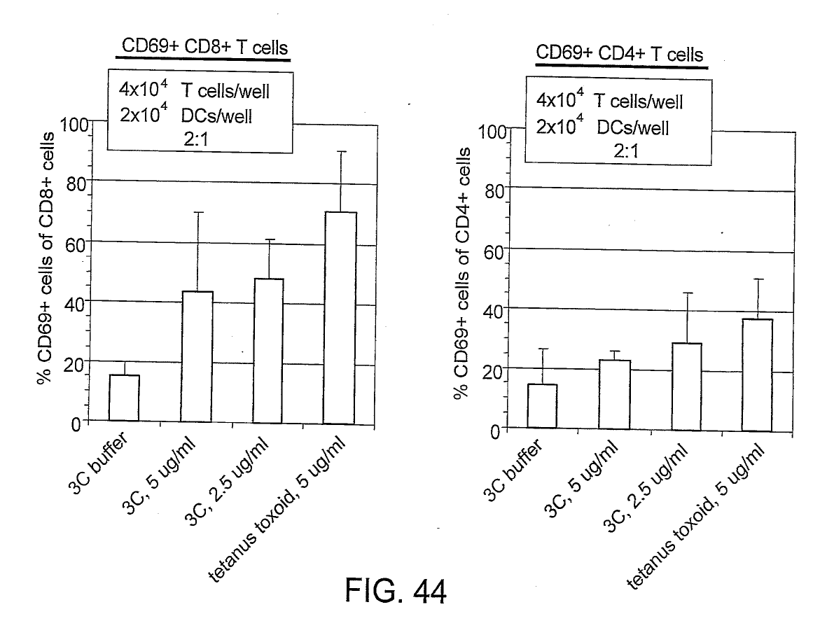

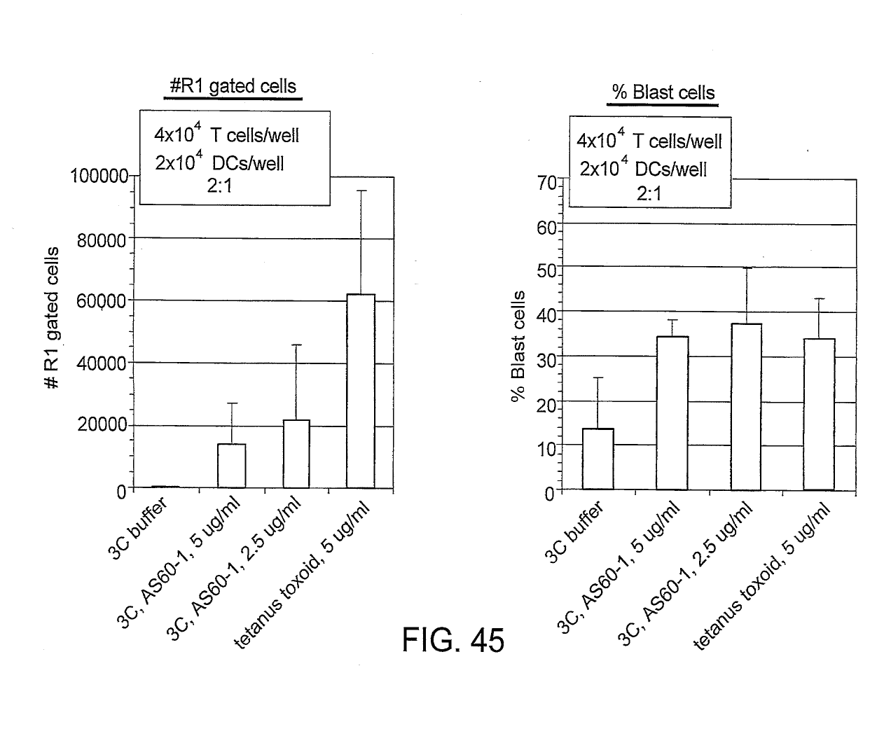

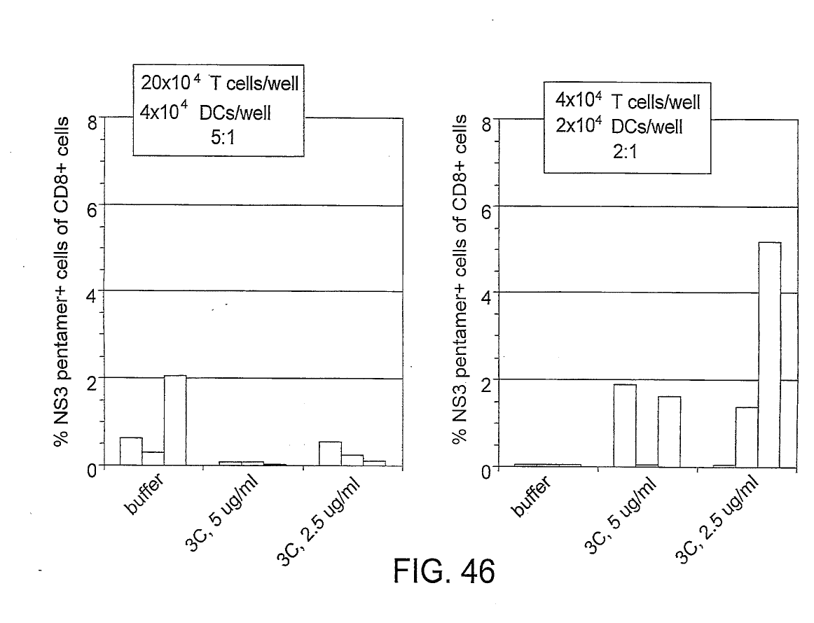

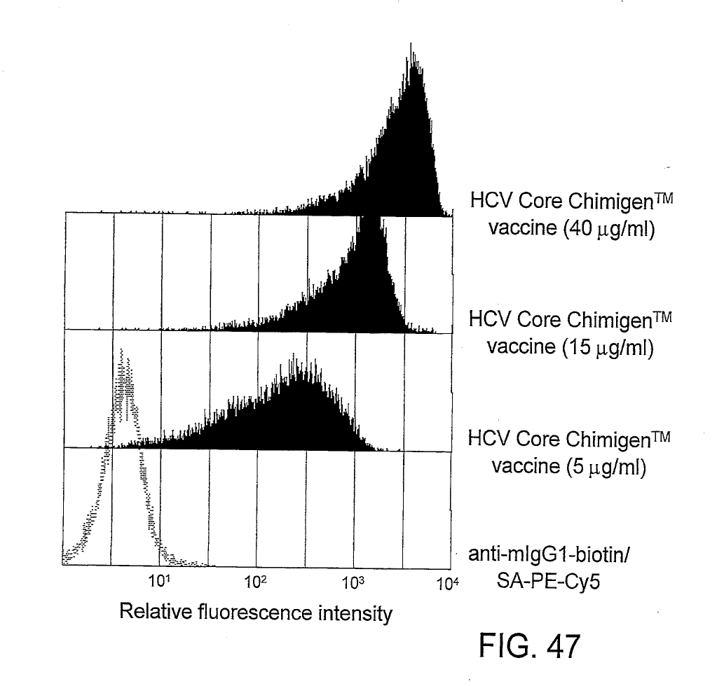

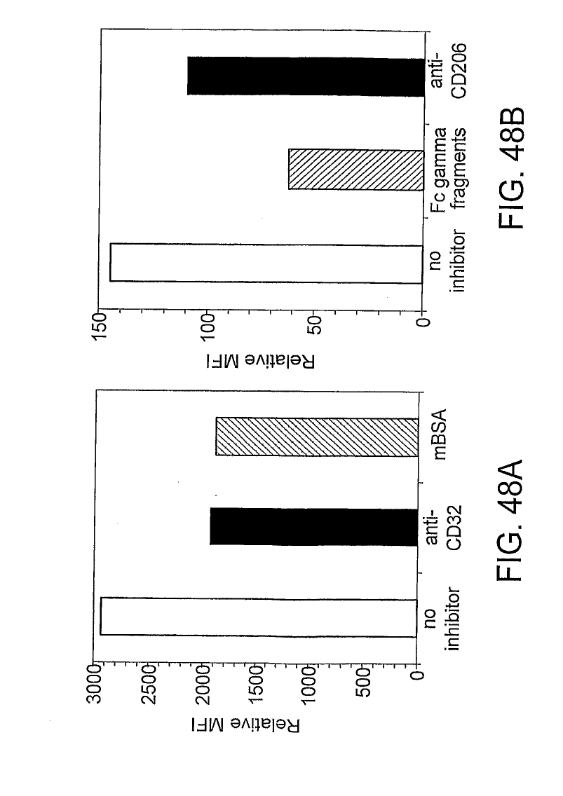

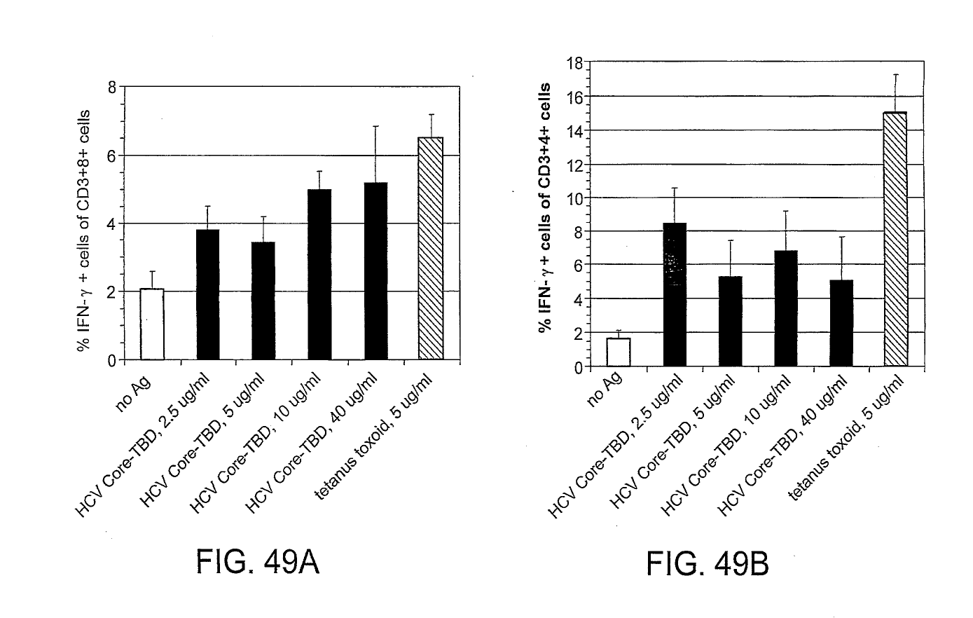

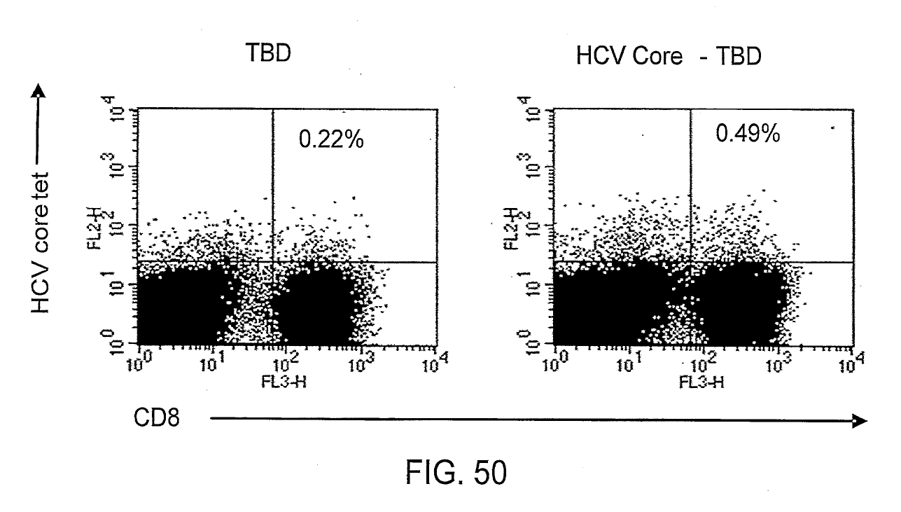

In order to clone NS5A Chimigen™ Vaccine into pFastBacHTa-gp64, the plasmid pFastBacHTa HCV NS5A Chimigen™ Vaccine (described above) was digested with Rsr II and Hind III. and the NS5A Chimigen™ Vaccine fragment was purified by agarose gel electrophoresis. The NS5A Chimigen™ Vaccine fragment was ligated to Rsr II and Hind III digested pFastBacHTa-gp64 plasmid to yield the pFastBacHTa-gp64 HCV NS5A Chimigen™ Vaccine (pFastBacHTa-gp64-NS5A-TBD) expression plasmid (IDAC accession no. 111006-04). The nucleotide sequence (SEQ ID NO:41) of the ORF in pFastBacHTa-gp64-NS5A-TBD and the amino acid sequence (SEQ ID NO:52) encoded by the ORE are shown in FIG. 4.

Construction of pPSC12-NS5A-TBD Chimigen™ Vaccine Expression Plasmid for Secretion

In order to facilitate the secretion of NS5A Chimigen™ Vaccine molecule, cloning into the plasmid pPSC12 (Protein Sciences Corporation) was performed. This plasmid has the signal peptide for the chitinase gene from the baculovirus Autographica californica nuclear polyhedrosis virus (AcNPV). Four PCR primers were required to clone a gene of interest into the transfer vector. The gene of interest was amplified using two unique primers (Primers 1 GTTTCTAACGCGTCGTACTACCATCACCATCAC (SEQ ID NO:15) and 2 CCGGGGTACCTTACAGCCCAGGAGAGTGGGAGAG (SEQ ID NO:16)). Two separate primers were required to amplify a polyhedron upstream region, containing the upstream polyhedron promoter and the signal peptide sequence (Primers 3 CTGGTAGTTCTTCGGAGTGTG (SEQ ID NO:17) and 4 GGTAGTACGACGCGTTAGAAACGGCGACCAAC (SEQ ID NO:18)). Finally, two outside primers (Primers 3 and 2, sequences above) were used in the critical overlap extension PCR. NS5A-TBD was amplified from pFastBacHTa-NS5A-TBD by PCR using primer 1 that contains a sequence that would anneal to the 5′ end of primer 4 and primer 2 that adds a unique 3′ Kpn I site for cloning into the vector. The upstream polyhedron region of pPSC12 was amplified with primer 3 and primer 4 which allowed it to anneal to the 5′ end of primer 1 during the overlap extension PCR. This upstream region also contains a unique NgoM IV site which is used for cloning into the vector. The upstream polyhedron promoter, the signal peptide sequence, and the desired gene were seamlessly fused by overlap extension PCR using primers 2 and 3. The full length fused product was digested with NgoM IV and Kpn I and the resulting fragment was ligated into an identically digested pPSC12 to generate pPSC12-NS5A-TBD. The nucleotide sequence (SEQ ID NO:42) of the ORF in pPSC12-NS5A-TBD and the amino acid sequence (SEQ ID NO:53) encoded by the ORF are shown in FIG. 5.

Construction of pFastBacHTa HCV NS3 Chimigen™ Vaccine Plasmid

DNA encoding NS3 was generated by PCR from the plasmid pCV-H77C template from amino acids 1027 to 1652 (nt 3420 to 5294) of the HCV polyprotein using the following primers. The final C-terminal 6 amino acids of NS3 were not included in the construct because those sequences are the target sequence for the serine protease activity of NS3. The 5′ terminus primer used was 5′-CCGGAATTCGCGCCCATCACGGCGTA-3′ (SEQ ID NO:19) containing an Eco RI restriction site and the 3′ terminus primer was 5′-CCGGACTAGTCC GGCCGACATGCATGTCATGAT-3′ (SEQ ID NO:20) containing Spe I restriction site. A double digestion with Eco RI and Spe I resulted in a product that was ligated with the plasmid pFastBacHTa-TBD to generate pFastBacHTa NS3-TBD.

Mutagenesis of pFastBacHTa HCV NS3 Chimigen™ Vaccine Plasmid Vector

Internal cleavage of the NS3 protein when expressed in insect cells, presumably mediated by cellular protease(s), has been reported by Shoji et al. [(1999) Virology 254:315-323] to occur at the arginine residue at 1488. Overlap extension (OE) PCR was used to generate a mutation of the amino acid arginine to alanine and thereby avoid such cleavage of the NS3 part of the NS3 Chimigen3 protein. Two NS3 DNA fragments were generated from the parent pFastBacHTa NS3-TBD plasmid. The 5′ NS3 fragment was generated with the primer 5′-CCGGAATTCGCGCCCATCACGGCGTA-3′ (SEQ ID NO:19) containing the Eco RI restriction site and the mutation primer (containing the arginine to alanine mutation) 5′-CTGCCAGTCCTGCCCGCGCGTTGAGTCCTGGAG-3′ (SEQ ID NO:21). The 3′ NS3 fragment was generated with the 5′ primer 5′-GGCAGGACTGGCAGGGGGAAGCCAGGCAT-3′ (SEQ ID NO:22) and the 3′ primer 5′-CCGGACTAGTCCGGCCGACATGCATGTCATGAT-3′ (SEQ ID NO:20) containing the Spe I restriction site. OE PCR was done with the 5′ and 3′ NS3 fragments, plus the two outside primers. A double digestion with Eco RI and Spe I resulted in a product that could be ligated with the plasmid pFastBacHTa-TBD to generate pFastBacHTa NS3mut-TBD (IDAC accession no. 111006-05). The nucleotide sequence (SEQ ID NO:44) of the ORF in pFastBacHTa NS3mut-TBD and the amino acid sequence (SEQ ID NO:55) encoded by the ORF are presented in FIG. 7. The predicted molecular weight of the protein is 98.3 KDa. For NS3mut alone, the NS3mut fragment was isolated by digestion with EcoR I and Spe I and cloned into pFastBac-HTa to generate pFastBacHTa-NS3mut

Construction of pFastBacHTa-gp64 HCV NS3mut Chimigen™ Vaccine Vector Plasmid

The pFastBacHTa HCV NS3mut-TBD plasmid was digested with Rsr II and Hind III restriction enzymes and the NS3mut-TBD fragment was cloned into Rsr II and Hind III digested pFastBacHTa-gp64 to generate pFastBacHTa-gp64-NS3mut-TBD (IDAC accession no. 111006-02). The nucleotide sequence (SEQ ID NO:45) of the ORF in pFastBacHTa-gp64-NS3mut-TBD and the amino acid sequence (SEQ ID NO:56) encoded by the ORF are presented in FIG. 8. The predicted molecular weight of the protein is 101.5 KDa. A clone of the mutated NS3mut-TBD fragment similar to that used to make pFastBacHTa-gp64-NS3mut-TBD (but lacking one spontaneous mutation and having another) was also ligated into pFastBacHTa-gp64 to generate a second clone of pFastBacHTa-gp64-NS3mut-TED. The nucleotide sequence (SEQ ID NO:43) of the ORF in the second clone of pFastBacHTa-gp64-NS3-TBD and the amino acid sequence (SEQ ID NO:54) encoded by the ORF are presented in FIG. 6.

Construction of pFastBacHTa HCV Multi-antigen Chimigen™ Fusion Protein Expression Vector Plasmid

To make the HCV multi-antigen vector plasmid the NS4B-NS5A sequences were first cloned. The DNA sequence encoding NS4B to NS5A was generated by PCR from the plasmid pCV-H77C using the primers 5′-GCOCACTAGTGTCTCAGCACTTACCGTACATC-3′ (SEQ ID NO: 23) for the 5′ terminus and 5′-CGGCGCGGCCOCCCGCAGCACACGACATG′TCCG-3′ (SEQ ID NO:24) for the 3′ terminus. PCR with these primers resulted in a product with unique restriction enzyme sites of a Spe I site at the 5′ end and a Not I site at the 3′ end. The PCR product was digested with Spe I and Not I and ligated into a Spe I and Not I digested pFastBacHTa-gp64 to generate pFastBacHTa-gp64 NS4B-NS5A. Next, the TBD portion was added to construct. The plasmids pFastBacHTa-TBD and pFastBacHTa-gp64 NS4B-NS5A were digested with Spe I and Hind III. The Spe I/Hind III digested TBD fragment was isolated and ligated to the digested pFastBacHTa-gp64 NS4B-NS5A to generate pFastBacHTa-gp64 NS4B-NS5A-TBD.

### Mutagenesis of NS3 Active Site Serine Residue

In NS3 the active site serine (ser1165) was mutated to alanine to abrogate the protease activity. Two NS3 fragments were created using four different primers, two nested and two complimentary to the 5′ and 3′ ends, by OE PCR with pFastBacHTa-gp64 NS3mut-TBD as template. The 5′ NS3 fragment was generated using the 5′ terminus primer (sense) 5′ CCGGAA′TTCGCGCCCATCACGGCGTA-3′ (SEQ ID NO:19), which contains the restriction enzyme Eco RI site and the mutation primer (containing the ser to ala mutation) (antisense) 5′ CAACAGCGGACCCCCCGCGGAGCCTTTCAAGTAG-3′ (SEQ ID NO:25). The 3′ NS3 fragment was generated using the 5′ terminus primer (sense) 5′GTCCGCTGTTGTGCCCCGCGGGACACG-3′ (SEQ ID NO:26) and the 3′ terminus primer (antisense) 5′-CCGGACTAGTCCGGCCGACATGCATGTCA-3′ (SEQ ID NO:27), which contains the restriction enzyme Spe I site. The full length NS3 (ser¹¹⁶⁵→ala) was generated by OE-PCR from the 5′ and 3′ fragments and the two outside primers. The resulting product with mutations at Arg 1488 to Ala and Ser 1165 to Ala is called NS3mutS. This fragment was cloned into pFastBacHTa-gp64 to generate pFastBacHTa-gp64 NS3mutS-TBD (IDAC accession no. 111006-001).

Construction of pFastBacHTa-gp64 HCV NS3-NS4B-NS5A Multi-antigen Fusion Protein Vector Plasmid

To make a construct that can be used for expression the fusion protein HCV NS3mutS-NS4B-NS5, the NS3mutS OE-PCR product was digested with the restriction enzymes Eco RI and Spe I. The digested NS3mutS was ligated into the Eco RI and Spe I digested plasmid pFastBacHTa-gp64 NS4B-NS5A-TBD to make pFastBacHTa-gp64 NS3-NS4B-NS5A-TBD (IDAC accession no. 111006-06), which is the pFastBacHTa-gp64 HCV Multi-antigen plasmid. The nucleotide sequence (SEQ ID NO:46) of the ORF in pFastBacHTa-gp64 NS3-NS4B-NS5A-TBD and the amino acid sequence (SEQ ID NO:57) encoded by the ORF are shown in FIG. 9.

Construction of pFastBacHTa-gp64 HCV NS3-NS5A Multi-antigen Fusion Protein Vector Plasmid

The DNA for HCV NS5A and TBD was generated by PCR from the template pFastBacHTa-64 HCV NS3-NS4B-NS5A-TBD. Chimigen™ Vaccine fusion protein expression vector plasmid. The 5′ primer for the PCR was (sense) 5′-GAGGGACTAGTGTCCGGTTCCTGGCTAAGGGAC-3′ (SEQ ID NO:28) containing the recognition site for the restriction enzyme Spe I. The PCR primer for the 3′ terminus was (antisense) 5′-CCGGTCTAGATTATGATCCICTAGTACTTCTCGAC-3′ (SEQ ID NO:29). The PCR product (NS5A-TBD) was gel purified and subsequently digested with Spe I and Hind III restriction enzymes. The plasmid pFasTBacHTa-gp64 NS3-NS4B-NS5A-TBD was digested with the restriction enzymes Spe I and Hind III, liberating a fragment consisting of the sequences encoding HCV NS4B-NS5A and the TBD. The resulting pFastBacHTa-gp64 HCV NS3 vector backbone was gel purified and ligated to the NS5A-TBD fragment to generate the expression plasmid pFastBacHTa-gp64 HCV NS3-NS5A Chimigen™ Vaccine (pFastBacHTa-gp64-NS3-NS5A-TBD) (IDAC accession no. 111006-03). The nucleotide sequence (SEQ ID NO:47) of the ORF in FastBacHTa-gp64-NS3-NS5A-TBD and the amino acid sequence (SEQ ID NO:58) encoded by the ORF are shown in FIG. 10.

Construction of pFastBacHTa HCV Core (1-177)-TBD Fusion Protein Plasmid and pFastBacHTa HCV Core (1-177)

The HCV core DNA sequences encoding amino acids 1-177 (nt 342-872) of the HCV polyprotein were amplified by PCR from pCV-H77C with 5′ primer CGGAATTCATGAGCACGAATCCTAAAC (SEQ ID NO:30) and 3′ primer GGACTAGTCCOAAGATAGAGAAAGAGC (SEQ ID NO:31). The primers used added unique 5′ EcoR I and 3′ Spe I sites. The PCR product was digested with EcoR I and Spe I and ligated into pFastBacHTa-TBD and pFastBac-HTa to generate the Chimigen™ vaccine construct pFastBacHTa HCV core (1-177)-TBD and pFastBacHTa HCV core(1-177), respectively. The nucleotide sequence (SEQ ID NO:48) of the ORF in pFastBacHTa HCV core (1-177)-TBD and the amino acid sequence (SEQ ID NO:59) encoded by the ORF are shown in FIG. 11.

The HCV core (1-177) was cloned into pFastBacHTa-gp64 and pPSC12, in order to produce the protein in a secreted form. For cloning into pFastBacHTa-gp64, the HCV core (1-177)-TBD fragment was isolated from pFastBacHTa HCV Core(1-177)-TBD by Rsr II and Hind III digestion and cloned identically digested pFastBacHTa-gp64 to generate pFastBacHTa-gp64 HCV core (1-177)-TBD.

For cloning into pPSC12, a similar scheme was used, as described for NS5A-TBD, except that primer 2 encodes a unique 3′ Bgl II site (AGTAAGATCTTTACAGCCCAGGAGAGTGGGAGAG; SEQ ID NO:32). The resulting construct is pPSC12-HCV core (1-177)-TBD.

Construction of pFastBacHTa HCV E1-TBD Fusion Protein Plasmid and pFastBacHTa-E1

The DNA sequence encoding amino acids 192 to 369 (914-1452) of the HCV polyprotein were amplified from pCV-H77C with 5′ primer CCGGAA′TTCTACCAAGTGCGCAATTCCT (SEQ ID NO:33) and 3′ primer GCGCACTAGTCCCTTCGCCCAGTTCCCCACC (SEQ ID NO:34) that add a unique 5′ EcoR I site and a unique 3′ Spe I site. The entire E1 open reading frame ends at amino acid 383 but the area between amino acids 370 and 383 is the signal sequence for E2 and was therefore not amplified. The PCR product was digested with EcoR I and Spe I and ligated into identically digested pFastBacHTa-TBD to generate the HCV E1 Chimigen™ construct pFastBacHTa-E1-TBD. To express E1 alone, the digested PCR product was cloned into EcoR I and Spe I digested pFastBac-HTa to generate pFastBacHTa-E1. The nucleotide sequence (SEQ ID NO:49) of the ORF in pFastBacHTa-E1-TBD and the amino acid sequence (SEQ ID NO:60) encoded by the ORF are shown in FIG. 12.

Construction of pFastBacHTa E2-TBD Fusion Protein Plasmid and pFastBacHTa-E2

The E2 sequences from amino acid 384 to 718 (nt 1494-2495 of the HCV polyprotein) were amplified by PCR from pCV-H77C with 5′ primer GCGCACTAGTCACCCACGTCACCGGGGGAAATG (SEQ ID NO:35) and 3′ primer GCGCGCGGCCGCCCGTACTCCCACTTAATGGC (SEQ ID NO:36) that add a unique 5′ Spe 1 site and a unique 3′ Not I site. The amino acids 719 to 746 are the signal sequence for p7 so was not included in construct. The PCR product was digested with Spe I and Not I and ligated to an identically digested pFastBacHTa-TBD to generate the HCV E2 Chimigen™ construct pFastBacHTa E2-TBD. The digested E2 was also cloned into pFastBac-HTa to generate pFastBacHTa-E2 for expression of E2 protein alone. The nucleotide sequence (SEQ ID NO:50) of the ORF in pFastBacHTa E2-TBD and the amino acid sequence (SEQ ID NO:61) encoded by the ORF are shown in FIG. 13.

Construction of pFastBacHTa-E1-E2-TBD Fusion Protein Plasmid and pFastBacHTa-E1-E2

A fusion of the E1 and E2 proteins was generated by subcloning the E1 sequence in pFastBacHTa-E1 into pFastBacHTa-E2. The pFastBacHTa-E1 plasmid was digested with Eco RI and Spe I and the fragment was cloned into Eco RI and Spe I digested pFastBacHTa-E2 to generate pFastBacHTa-E1-E2. To make the E1-E2 Chimigen™ construct, pFastBacHTa-E1-E2 was digested with Eco RI and Not I and cloned into identically digested pFastBacHTa-TBD to generate pFastBacHTa-E1-E2-TBD. The nucleotide sequence (SEQ ID NO:51) of the ORF in pFastBacHTa-E1-E2-TBD and the amino acid sequence (SEQ ID NO:62) encoded by the ORE are shown in FIG. 14.

### Production of Recombinant Baculoviruses in the Bac-to-Bac® Expression System

Transformation of E. coli

Ligated plasmids were used to transform E. coli DH5α and the plasmids were isolated by standard protocols. Sequence and open reading frames were verified by sequencing and used for the production of recombinant baculoviruses.

### Transposition

The generation of recombinants is based on the Bac-To-Bac® cloning system (Invitrogen) that uses site-specific transposition with the bacterial transposon Tn7. This is accomplished in E, coli strain DH10Bac. The DH10Bac cells contain the bacmid pMON14272, which confers kanamycin resistance, and a helper plasmid (pMON7124) that encodes the transposase and confers resistance to tetracycline.

The gene of interest is cloned into the pFastBac plasmid that has mini-Tn7 elements flanking the cloning sites. The plasmid is transformed into the E. coli strain DH10Bac, which has a baculovirus shuttle plasmid (bacmid) containing the attachment site of Tn7 within a LacZα gene. Transposition disrupts the LacZα gene so that only recombinants produce white colonies and thus are easily selected.

The advantage of using transposition in E. coli is that single colonies contain only the recombinant. The recombinant bacmids are isolated using standard plasmid isolation protocols and are used for transfection in insect cells to generate baculoviruses that express recombinant proteins.

Donor plasmids and pFastBacHTa-gp64 Chimigen™ vaccine vectors were used for the site-specific transposition of the cloned gene into a baculovirus shuttle vector (bacmid). The recombinant pFastBacHTa-gp64 plasmid with the gene of interest was transformed into DH10Bac cells for the transposition to generate recombinant bacmids. A 40 μL aliquot of competent DH10Bac cells was thawed on ice, the pFastBacHTa-gp64 based plasmids were added and transformation was performed by electroporation. The transformation mixture was added to 1 mL of SOC media and incubated for 4 hours at 37° C. The transformed cells were serially diluted with LB to 10⁻¹ and 10⁻² and 100 μL of each dilution was plated on LB agar plates supplemented with kanamycin (50 μg/mL), gentamicin (7 μg/mL), tetracycline (10 μg/mL), X-gal (200 μg/mL), and IPTG (40 μg/mL) and incubated for at least 36 hours at 37° C.

Gentamicin resistance was conferred by the pFastBacHTa-gp64 plasmid and X-gal and IPTG were used to differentiate between white colonies (recombinant bacmids) from blue colonies (non-recombinant). The white colonies were picked and inoculated into 2 mL of LB supplemented with kanamycin (50 μg/mL), gentamicin (7 μg/mL), and tetracycline (10 μg/mL) and incubated overnight at 37° C. with shaking. A sterile loop was used to sample a small amount of the overnight culture and the sample was streaked onto a fresh LB agar plate supplemented with kanamycin (50 μg/mL), gentamicin (7 μg/mL), tetracycline (10 μg/mL), X-gal (100 μg/mL), and IPTG (40 μg/mL) and incubated for at least 36 hours at 37° C. to confirm a white phenotype. Recombinant bacmids were isolated by standard protocols [Sambrook et al. (2001) In Molecular Cloning, A Laboratory Manual. Cold Spring Harbor Press], the DNA sample was dissolved in 40 μL of TE (10 mM Tris-HCl pH 8, 1 mM EDTA) and used for transfections.

### Transfection: Production of Recombinant Baculovirus

In order to produce recombinant baculoviruses, the relevant bacmid was transfected into Sf9 insect cells. Sf9 cells (9×10⁵) were seeded into each well of a 6 well cell culture dish (35 mm wells) in 2 mL of ESF 921 and allowed to attach for at least 1 hour at 27° C. Transfections were carried out using Cellfectin® reagent with the protocols provided by the supplier of the Sf9 cells. Following transfection, the cells were incubated at 27° C. for 72 hours. The medium containing baculovirus was collected and stored at 4° C. in the dark.

The efficiency of the transfection was verified by checking for production of baculoviral DNA. The isolated baculovirus DNA was subjected to PCR to screen for the inserted gene of interest. The expression of the heterologous protein in the cells was verified by SDS-PAGE and Western blots using the 6×His tag-HRP conjugated monoclonal antibody or anti-mouse IgG (Fc specific) horseradish peroxidase conjugated antibody as the probe.

### Amplification of the Recombinant Baculovirus Stock

Once generation of the baculovirus and the expression of the desired protein were confirmed, the virus concentration was amplified to produce a concentrated stock of the baculovirus that carried the gene of interest. In all the protocols described herein, the standard practice of amplifying the baculovirus at least twice was followed. After the second round of amplification, the concentration of the generated baculovirus was quantified using the baculovirus titering assay (Expression Systems). The most appropriate concentration of the virus to infect Sf9 cells and the optimum time for the production of the desired protein were also established. The protocols for the expression for both monolayer as well as suspension culture of Sf9 cells were developed according to standard procedures.

### Baculovirus Titering Assay

All viral stocks are titered using the Expression Systems baculovirus titering assay. Viral stocks were diluted serially from 10⁻¹ to 10⁻⁴. A 100 μL aliquot of each of the diluted samples was added to wells of a Costar Low Attachment3 96 well, plate. Then, 100 μL of Sf9 cells at a concentration of 2×10⁶ cells/mL was added to each well and the plate incubated for 18 hr at 27° C. in an orbital shaker incubator at 200-250 rpm.

Following incubation, gp64-PE conjugated antibody was diluted 1:200 and the isotype control (IgG_{2A}-PE) was diluted 1:10. The plate was centrifuged for 3 minutes at 1800 rpm. The media was removed by inversion of the plate and 50 μL of the gp64-PE conjugated antibody or 50 μL of the isotype control was added to the wells. The plate was then incubated for 20 min at 4° C. in the dark.

The cells were washed by adding 150 μL cold PBS to each well and centrifuging the plate down as described above. Next, 200 μL of cold PBS was added to each well followed by another spin and finally 200 tit of PBS/0.1% BSA was added to each well to re-suspend the cells and transfer to FACS tubes for analysis. The isotype control was used to set gates on the fluorescence flow cytometer. The viral titer was determined by inserting percentage of the cells population that were positive for expression into the provided Excel spreadsheet and producing a standard curve based on the control virus.

### Optimization of Protein Expression

Chimigen™ Protein expression was optimized over a range of MOIs and times. Four 50 mL cultures of Sf9 cells in ESF 921 were seeded at 2×10⁶ cells/mL and infected with MOIs of 0.5, 1, 5 and 10 and 1 mL of culture was harvested after various time points post infection. Sampled cultures were centrifuged at 12000×g for 1 min and the supernatant and cells separated. Cells and supernatant were immediately prepared for SDS-PAGE analysis. The cells were resuspended in 500 μL of PBS, and 150 μL of the suspension was added to 40 μL 5× loading buffer and 10 μL 20× reducing agent. Also, 150 μL of supernatant was mixed with 40 μL 5× loading buffer and 10 μL 20× reducing agent. Samples were boiled for 5 min and loaded onto a 12% SDS-PAGE gel for Western blot analysis. Protein production was assessed to be best for NS5A Chimigen™ protein at 36 hr and at a MOI of 0.5, for the NS3 Chimigen™ protein at 48 hr at a MOI of 2, and for the multi-antigen Chimigen™ protein at 36 hr at a MOI of 1.5.

### Purification of Intracellular Chimigen™ Vaccine

### Large-Scale Expression of NS5A Chimigen™ Protein and Preparation of Cell Lysate

Five litres of Sf9 cell culture at a density of ˜2×10⁶ cell/ml in ESF 921 medium were infected with baculovirus at a MOI of 0.5. Cells were harvested ˜36 hr after the start of infection when the cell viability was ˜95%. Longer expression times resulted in increasing loss of cell viability and an intense degradation of the NS5A Chimigen™ Protein. Infected cells were collected by centrifugation and stored at −80° C. until use.

Frozen pellets from 1 L infected cell culture were resuspended rapidly by vortexing on ice in 200 ml lysis buffer containing a high concentration of Tween 20 (6M GuHCl, 50 mM NaH₂PO₄, 0.5 M NaCl, 1% Tween 20, 10 mM σ-mercaptoethanol, pH 8.0). A high concentration of Tween 20 was necessary for efficient binding of the NS5A Chimigen™ Protein to the Ni-NTA resin. The resuspended suspension was sonicated on ice 3×1 min with 2 min intervals in between each sonication pulse. The sonicated lysate was then stirred for 2 hr at room temperature. The stirred lysate was cleared by centrifugation (˜27,000×g for 15 min. at 10° C.) and the supernatant used for affinity chromatography on Ni-NTA superflow.

### Expression of NS3 Chimigen™ Protein and Preparation of Cell Lysate

Recombinant baculovirus encoding for HCV NS3 Chimigen™ Protein of standardized multiplicity of infection (MOI) was used to infect Sf9 insect cells for protein expression. Sf9 cells were seeded at a density of 6×10⁵ cells/mL in 500 mL of ESF 921 media in a 2 L Erlenmeyer flask. The cell culture was incubated at 27.5° C. with shaking at 120 rpm until the cell density reached 2-3×10⁶ cells/mL. For the HCV NS3 Chimigen™ Protein, cells were infected at a MOI of 2 for 48 hr. A Western Blot analysis on cell lysate was carried out for monitoring the expression of the protein of interest. The cells were harvested by centrifugation at 3,000 rpm (1593×g, JA10, Beckman Coulter Avanti™ J 25) for 10 min at 4° C. and fresh cell pellet was used for the purification of the recombinant protein. Alternatively, cell pellets were re-suspended with the conditioned media, distributed into 50 mL Conical tubes (250 mL cell culture for each tube), spun at 2,200 rpm for 8 min at 4° C. in the Beckman GS-6R centrifuge. Cell pellets were snap frozen in liquid nitrogen and stored at −80° C.

A frozen cell pellet (equivalent 2×500 mL of cell culture media) was resuspended on ice in 200 mL of ice cold lysis buffer (6M GuHCl, 150 mM NaCl, 20 mM Tris-HCl, pH 8.00) by sonication for 1 min, 78 W (setting 6.5). The mixture was transferred to 250 mL glass beaker and sonicated four more times for 1 min, 78 W each time, with 5 min cooling intermissions. The mixture was moved to room temperature and CTAB was added to a final concentration of 1% (w/v). The pH was checked and adjusted to pH 8.00 and the lysate was incubated for 2 hrs. The lysate was centrifuged for 30 min at 15,000 rpm (27,000×g) at 10° C. using JA 25.50 rotor in a Beckman Avanti J-25 centrifuge and the supernatant was subjected to Ni-NTA affinity chromatography.

### Large-Scale Expression of the Multi-Antigen Chimigen™ Protein and Preparation of Cell Lysate

Four litres of ESF921 medium was transferred into a Cellbag and was warmed to 27.5° C. on a Wave Bioreactor System 2/10 EH. One litre of Sf9 cell culture at 6×10⁶ cells/mL was added to the Cellbag. The bag was then incubated at 27.5° C. with injection of air at 0.3 L per minute and was rocked at 130 rpm. When the density of the cells reached to 2×10⁶ cells/ml, recombinant baculovirus was inoculated at MOI of 1.5. At 36 hour after infection, the bag was chilled on ice and cells were harvested by centrifugation at 4500×g for 10 minutes at 4° C. The cell pellet was suspended in ice-cold PBS and then transferred into a 50 mL conical tube (pellet from 300 mL culture per tube). The cell pellet was recovered by centrifugation at 2800×g for 15 minutes at 4° C. The pellet was frozen immediately in liquid nitrogen and was stored in −80° C. freezer until use.

The frozen Sf9 cell pellet from 250 ml culture was suspended in 20 ml 1×PBS, 1% Tween 20, 50 mM DTT, 5 mM EDTA, pH 8.0 and incubated on ice for 30 min. The pH of the lysate was adjusted to pH 12.0 with NaOH and stirred at room temperature for 30 min. The pH was lowered to 8.0 with HCl and centrifuged for 30 min at 39191×g. The supernatant was removed and the pellet was suspended in 20 ml 1×PBS, 1% Tween 20, 10 mM DTT, 1 mM EDTA, pH 8.0. The pH was raised to 12.0 and reduced to 8.0, as described above. The supernatants were pooled and dialyzed against 20 mM Tris, 0.05% Tween 20, 0.1 mM EDTA, 10 mM θ-mercaptoethanol, pH 8.0 for use in size exclusion and hydrophobic interaction chromatography.

For Ni-NTA affinity chromatography, frozen Sf9 cell pellet from 500 ml culture was suspended in 50 mL ice-cold Lysis buffer (6M Guanidine-HCl, 50 mM Sodium Carbonate, 20% Ethanol, pH 10). The cell lysate was sonicated five times on ice by Sonicator 3000 (Misonic Inc.) at 80 W for 1 minute. Tween 20 was added into the lysate (final concentration 1%) and the lysate was stirred for 2 hours at room temperature. Insoluble particulates in the lysate were removed by centrifugation at 39,191×g for 30 minutes at 4° C. and subjected to Ni-NTA affinity chromatography.

### Expression of HCV Core Chimigen™ Protein and Preparation of Cell Lysate

HCV core Chimigen™ was expressed in two systems. Recombinant viruses were generated with co-transfection of pPSC12-HCV core (1-177)-TBD and a linearized baculovirus genome in Sf9 cells, plaque purified and amplified. After optimization, Sf9 cultures were infected at MOI of 5 and harvested after 50 hrs of incubation at 27.5° C. Recombinant viruses were also generated using the Bac-to-Bac® system by transfection of E. coli DH10Bac cells with pFastBacHTa-gp64 HCV core (1-177)-TBD. Recombinant bacmids were isolated and used to transfect Sf9 cells to make recombinant baculoviruses. Sf9 cultures were infected at an MOI of 5 for 49 hrs at 27.5° C. before harvesting by centrifugation. Lysates were prepared in essentially the same manner described above for other Chimigen™ Proteins and subjected to Ni-NTA affinity chromatography.

### Ni-NTA Affinity Chromatography

The cell lysate was loaded onto a Ni-NTA superflow column (10 ml resin bed volume per 2.5 L cell culture pellet) that had been equilibrated with 10 bed volumes of lysis buffer. The column was washed with a wash buffer containing reduced % Tween 20 (6M GuHCl, 50 mM NaH₂PO₄, 0.5 M NaCl, 0.1% Tween 20, 10 mM θ-mercaptoethanol, pH 8.0) until A₂₈₀<0.01 and then with the same wash buffer containing 15 mM imidazole. Target protein was then eluted with 10 ml bed volumes of elution buffer (6M GuHCl, 50 mM NaH₂PO₄, 250 mM imidazole, 0.1% Tween 20, 10 mM θ-mercaptoethanol, pH 8.0) in 1 bed volume fractions. Fractions containing eluted protein were pooled and dialysized against dialysis buffer 3×4 L (8M Urea, 20 mM Tris, 0.1% Tween 20, 25 mM ethylenediamine, 10 mM θ-mercaptoethanol, pH 8.5). All urea-containing buffers were made with deionized urea to prevent carbamylation of the protein. Urea was deionized with Ainberlite® MB-1 (Supelco, Pa., USA) (10 g/L/hr) and the cyanate scavanger ethyenediamine was added (25 mM final) to the buffer,

### Ion Exchange Chromatography

The dialyzed NS5A Chimigen3 Protein-containing sample obtained by Ni-NTA affinity chromatography was next passed over a Toyopearl® Super Q3 resin column (2.5 ml bed volume/2.5 L cell culture pellet) that had been equilibrated in dialysis buffer. The ion exchange column was washed with ion exchange wash buffer (8M Urea, 20 mM Tris, 0.05% Tween 20, 25 mM ethylenediamine, 10 mM θ-mercaptoethanol, pH 8.5) until A₂₈₀<0.01. Proteins were then eluted from the column with 10 bed volumes of wash buffer containing increasing concentrations of salt (75 mM, 150 mM and 500 mM NaCl). One bed volume fractions were collected. The NS5A Chimigen™ Protein eluted off predominantly in the 150 mM NaCl fractions. A contaminating protein of slightly lower MW eluted off at 75 mM NaCl. Eluted protein fractions were pooled and dialyzed immediately against final dialysis buffer (150 mM NaCl, 10 mM NaH₂PO₄, 0.05% Tween 20, pH8.5) at 4° C. 2 L per dialysis, with five changes dialysis buffer. Dialyzed proteins were filtered through a 0.2 μm filter that had been pre-wet to prevent proteins sticking to it. Purified NS5A Chimigen™ Protein was stored at 4° C.

For further purification of the NS3 Chimigen3 Protein, CM-Sepharose3 Fast Flow matrix was equilibrated with 8M Urea (de-ionized), 25 mM NaH₂PO₄, 5 mM Ethylenediamine, 0.05% (v/v) Tween 20, 10 mM DTT, pH 6.50. Protein was eluted using a linear gradient (0 to 0.6 M) of sodium chloride in the same buffer at a flow rate of 1 mL/min. Fractions containing the protein (25-50 mM NaCl) were pooled.

The multi-antigen Chimigen3 Protein containing sample captured by Ni-NTA column was further purified by HiTrap3 Q XL 1 ml column using AKTAexplorer3 100 FPLC system. The protein in elution buffer from Ni-NTA affinity chromatography was concentrated by an Amicon Ultra-15 (MWCO 30,000 Da) and then the buffer was exchanged to Buffer A (8M Urea, 50 mM Sodium. Carbonate, 25 mM ethylendiamine, 1% Tween 20, pH 10). Protein was loaded onto a HiTrap Q™ XL column, equilibrated with 50 ml of Buffer A, at flow-rate of 60 mL/hour. The column was washed with Buffer A until A₂₈₀ of elution is below 0.01. Proteins were eluted by linear gradient elution, from 100% Buffer A to 100% Buffer B, (Buffer A with 1M Sodium Chloride) in 20 column volumes. HCV multi-antigen Chimigen™ Protein was eluted in the flow-through fractions and in fractions eluted between 40 and 50% Buffer B.

### Size Exclusion Chromatography

Superdex3 200 preparative grade was packed in a Tricon3 column 10/300 (1×30 cm, Pharmacia Biotech) under the pressure of 3 MPa using AKTAexplorer 100 FPLC system (GE healthcare). The column was washed with 100 ml of 6 M Guanidine, 50 mM Sodium Carbonate, pH 10. 0.5 mL of the lysate containing the Chimigen3 multi-antigen protein was loaded onto the Superdex 200 column. Protein was eluted by flow rate at 30 mL/hour and 0.5 mL fractions were collected. Protein elution was monitored by the absorbance at 280 nm.

### Hydrophobic Interaction Chromatography

Phenyl-650C Toyopearl® (0.5 mL, TOSOH Corp.) was packed into a Poly-prep column (Bio-Rad). The column was equilibrated with 20 mL HIC binding buffer (0.1 M Tris, 2 M Sodium Chloride, pH 8). Sodium Chloride at final concentration of 2 M was added into the 0.5 mL lysate containing the Chimigen3 Multi-antigen protein and the extract was diluted with 3.5 mL HIC binding buffer. Insoluble particulates were removed by centrifugation at 18,000 rpm (39,191×g, by JA25.50 rotor, Beckman Coluter Avanti™ J-25 centrifuge) for 20 minutes. The supernatant was loaded onto the column at flow rate of 30 mL/hour by gravity flow. The column was then washed with 10 mL HIC binding buffer and protein, bound on the column, was eluted with 5 mL HIC elution buffer (8 M Urea, 50 mM Ethylendiamine, 0.5% Tween 20, pH 10.5).

### Biochemical Evaluation of Purified Chimigen™ Proteins

The concentrations of proteins were estimated using the Micro BCA3 protein assay reagent kit in a microplate procedure according to the protocol provided by the manufacturer.

For SDS-PAGE analysis, aliquots of purified proteins were denatured by adding 5× protein loading buffer and 20× reducing agent and boiled for 5 mins. Denatured proteins were separated on 12% SDS polyacrylamide gels and the gels were stained with PageBlue3 under the conditions provided by the manufacturer.

For Western blot analysis, proteins were separated by 12% SDS-PAGE and electroblotted onto Hybond3 ECL3 nitrocellulose membranes using a buffer containing 48 mM Tris base, 39 mM glycine, 20% methanol and 0.0375% SDS. The membranes were incubated first in blocking buffer (1% skim milk, 0.1% Tween 20 in PBS) for 1 hr at room temperature. Antibodies for detection were diluted in blocking buffer to the desired concentration. The membranes were incubated with the diluted antibodies for 1 hr at room temperature with constant mixing. After incubation with each antibody, the membrane was washed three times with blocking buffer for 10 min per wash at room temperature. Detection of proteins was performed by chemiluminescence with the ECL3 Western blotting detection kit and exposure to Kodak Biomax XAR X-ray film.

For the qualitative detection of glycosylation of proteins, the Pro-Q® Emerald 300 Glycoprotein Gel and Blot Stain Kit developed by Molecular Probes were used. This kit can be used for detection of carbohydrates on proteins separated by SDS-PAGE in gels or on blots. The stain is compatible with most total protein stains and if desired, analysis by mass spectrometry. A bright green-fluorescent signal is produced when the stain reacts with periodate-oxidized carbohydrate groups, detecting as little as 0.5 ng of glycoprotein per band. The stain is a modification of periodic acid and Schiff methods and the manufacturer claims a 50-fold greater sensitivity level. Included in the kit are CandyCane™ molecular weight standards. The standards consist of alternating glycosylated and non-glycosylated proteins serving as positive and negative controls respectively. Following the SDS-PAGE of the protein sample, the gel was fixed in 50% MeOH and 5% acetic acid overnight. The gel was washed twice for 20 minutes in 3% glacial acetic acid, followed by glycan oxidation in the oxidizing solution periodic acid for 30 minutes. The gel was washed three times for 20 minutes with 3% glacial acetic acid followed by staining in fresh Pro-Q3 Emerald 300 staining solution for a maximum of 120 minutes in the dark. An additional two 20 minutes washes in 3% glacial acetic acid in the dark is required before imaging. The excitation/emission max of the stain is 280/530 nm with the most optimal visualization at ˜300 nm. The gel was visualized and scanned using a GeneGenius (Syngene) transilluminator and corresponding software.

### Immunological Characterization of Chimigen™ Vaccines

Human PBMCs (Peripheral Blood Mononuclear Cells)

PBMCs were obtained by Ficoll-Hypaque gradient centrifugation of a leukapheresis preparation from non-HCV-infected individuals having the HLA-A2 haplotype (Biological Specialty Corporation). PBMCs were stored in liquid nitrogen at 3×10⁷ cells/cryovial in freezing media (50% Human AB serum, 40% AIM-V®, and 10% DMSO).

Isolation and Differentiation of Monocytes to Immature DC (Dendritic Cells)

PBMCs were cultured on 100 mm tissue culture plates (BD Biosciences) for 1 hr at 37° C. in AIM V® media with 2.5% matched serum. Following culture, non-adherent cells were removed and the plate washed with AIM V® media. The adherent cells were then cultured with 2 mL of AIM V®/2.5% matched serum containing IL-4 and GM-CSF (1000 IU/mL of each).

Binding of HCV Chimigen™ Proteins to immature DCs Immature DCs were obtained from culturing monocytes in the presence of IL-4 and GM-CSF for 24-72 hr. Following culture the cells were harvested, washed once with AIM V® media containing 2.5% matched serum, followed by two washes with Dulbecco's phosphate buffered saline (Invitrogen) containing 0.1% (w/v) BSA (PBSB). The cells were used to evaluate the binding and internalization of Chimigen™ Protein. The phenotype of the immature DCs was assessed by labeling for various cell surface markers including CD64, CD32, CD16, CD206, HLA-ABC, HLA-DR, CD14, CD11c, CD86, CD80, CD40, CD83, CD19, CD3, and CD4.

For the binding assay, all steps were performed at 4° C. with washes following the incubations. Cells were incubated for 60 min in PBSB with various concentrations of Chimigen™ Protein or the corresponding dialysis buffer (2×10⁵ cells/well in 96-well v-bottom plates in a volume of 25 μL). Protein binding was detected by incubation of the cells with biotinylated anti-mouse IgG1 or anti-6×His antibody in PBSB for 20 min, followed by SA-PE-Cy5 for 20 min. Cells were resuspended in PBSB containing 2% paraformaldehyde (PF). In experiments using NS5A Chimigen™ Protein, the binding was detected with a rabbit anti-NS5A polyclonal antibody, goat anti-rabbit IgG-biotin, SA-PE-Cy5 combination. Cells were resuspended in PBSB containing 2% paraformaldehyde (PF) and cell binding assessed by fluorescence flow cytometry (FFC).

Characterization of DC Receptors for Chimigen™ Vaccines Using Inhibitors of Binding

Immature DCs were incubated for 60 min at 4° C. in PBSB with anti-CD32 mAb (IgG2b isotype), anti-CD206 (IgG1 isotype), or isotype control mouse IgG2b or IgG1 mAbs. Subsequently, the cells were incubated with Chimigen™ Vaccines in PBSB for 60 min at 4° C. Following washes, the binding to the cells was detected by FFC analysis using either biotinylated anti-mouse IgG1 mAb or biotinylated anti-6×His mAb followed by SA-PE-Cy5.

Fluorescence Flow Cytometry (FFC) Analysis

Cells were acquired with a FACSCalibur fitted with CellQuest Pro acquisition and analysis software (BD Biosciences). A gate was set on the viable cell population as determined by the FSC and SSC scatter profile and >20,000 events were acquired. The percentage of specific positive cells was calculated as: (% positive cells test sample−% positive cells control)/(100−% positive cells of control)×100. The relative mean fluorescence intensity (MFI) was determined as: MFI of the test sample−MFI of the control sample.

Antigen Presentation Assays (APAs)

APAs are used to measure the immune response of T cells to antigen presented by APCs. The assays quantify functional T cell immune responses and the ability of antigen-loaded mature DCs to induce proliferation of antigen-specific T cells. The procedure includes differentiating PBMC-derived monocytes to immature DCs, loading the immature DCs with antigen (Chimigen™ Protein or TT), differentiating the immature DCs to mature DCs, and then culturing the mature antigen-loaded DCs together with autologous naïve T cells. For activation and proliferation assays, T cells were analyzed after 7 days of culture. For analysis of T cell function and specificity, T cells were stimulated two additional times with antigen-loaded mature DCs and the production of TNF-γ, TNF-α, granzyme B (grB), and perforin (pfn) assessed. Specificity of T cells to antigens was assessed with specific MHC class I tetramers or pentamers.

Generation of Antigen-Loaded Mature DCs

Immature DCs were generated as described above and incubated for 8 hr with antigen or buffer (control). The cells were then cultured for 16 hr with the maturing agents poly IC (20 μg/mL), recombinant human (rh) IL-113 (10 ng/mL), rhTNF-α (10 ng/mL), rhIL-6 (10 ng/mL), rhIFN-αA (1000 U/mL), and rhIFN-γ (1000 U/mL). The extent of maturation of the DCs was assessed by phenotype analysis. The cells were labeled for various cell surface markers including CD64, CD32, CD16, CD205, CD206, CD209, HLA-ABC, HLA-DR, CD14, CD11c, CD86, CD80, CD83, CD40, CD19, CD3, CD8, and CD4. The matured antigen-loaded DCs were washed and cultured with T cells.

Isolation of Human PBMC (Peripheral Blood Mononuclear Cells)-Derived T Cells

T cells were isolated from PBMCs by negative selection using a Dynal Biotech T cell negative selection kit (Invitrogen) following the manufacturer's procedure. Matched sera were used in place of BSA and FBS. The phenotype of the isolated cells was assessed by phenotype labeling for a variety of cell markers. T cells (CD3+ cells) comprised greater than 98% of the isolated population. The T cells were either labeled with CFSE (see below) or added directly to cell culture with DCs.

CFSE Labelling of T Cells

Freshly isolated T cells (1-5×10⁷ cells) were suspended in 500 μl of PBSB and mixed with 500 μl of a freshly prepared 10 μg/ml working stock solution of CFSE. Following an incubation for 10 min at 37° C. the cells were washed extensively with serum containing media (AIM V0/10% matched serum) to remove unincorporated CFSE. CFSE labeling of T cells was confirmed by FFC.

Culture of Human PBMC-Derived T Cells

T cells were incubated with antigen-loaded mature DCs at ratios of 1-20×10⁴ T cells to 1-5×10⁴ DCs per well in AIM V®/2 5% matched serum. For the T cell activation and proliferation APA experiments, T cells were harvested after 4 days and 7 days of culture (see below). For the T cell function and specificity APA experiments, T cells were cultured for 7 days and then restimulated with antigen-loaded mature DCs and cultured for an additional 7 days. The 14-day cultured T cells were then split into two to groups (intracellular cytokine (ICC) plate and tetramer plate) and stimulated a third time with antigen-loaded DCs, Brefeldin A (BD Biosciences) at 1 μg/mL was added to the wells of the ICC IFN-γ plate and the cells cultured for 6 h. The expression of IFN-γ, TNF-α, grB, and pfn was assessed as outlined below. Tetramer analysis was performed five-six days following stimulation as outlined below.

T Cell Activation and Proliferation Analysis

For the activation/proliferation APA, T cells were harvested after 4 or 7 days of culture with antigen-loaded DCs. The T cells were assessed for the expression of CD69 (early activation marker) and CFSE intensity (degree of proliferation). Harvested cells were labeled with anti-CD3-PE, anti-CD8-PE-Cy5, and anti-CD69-APC. Using buffer control samples the population of T cells that had not undergone any doubling was identified. This population labeled with a high degree of fluorescence detected in the FL1 channel and was designated as CFSE^{hi}. Cell populations that had undergone one division had half of the MFI of the CFSE^{hi} population. Similarly, populations that had undergone two divisions had approximately 25% (4 times less) MFI of the CFSE^{hi} population. Cells with a CFSE fluorescence lower than the CFSE^{hi} fluorescence were designated as CFSE^{lo}. Some of cell populations had near background FL1 channel fluorescence and could be designated CFSE− (CFSE negative). However for purposes of the experiments outlined here T cells were considered CFSE^{hi} (no cell divisions) or CFSE^{lo} (at least one cell division).

T cell activation was quantified by assessing the expression of CD69. In some experiments T cell blasts were quantified by gating on the population of high FSC and SSC intensity CD3+ T cells. Thus relative number of blast cells in a cell population was expressed (for these studies) as the proportion of cells with a larger diameter (FSC^{hi}) and with greater cellular complexity (SSC^{hi}) compared with the small (G0) resting cells in the population.

Detection of Intracellular IFN-α, grB and pfn

The production of IFN-γ and TNF α and the expression of the serine protease granzyme B (grB) [Lobe et al, (1986) Science 232:858-861] as Well as the pore-forming Protein perforin (pfn) [Hameed et al. (1992) Am. J. Pathol. 140:1025-1030] were quantified using a standard ICC (intracellular cytokines) protocol (13D Biosciences). In brief, this consisted of labeling the cells with specific fluorochrome conjugated mAbs for detection of CD3 (anti-CD3-APC) and CD8 (anti-CD8-PE-Cy5), followed by fixing and permeabilization. The cell samples were then divided into two samples, one of which was incubated with anti-IFN-γ-PE antibody and anti-grB-FITC antibody and the other with anti-TNF-α-PE and anti-perforin-FITC. On average between 20,000-100,000 cells per sample were acquired using a BD FACSCalibur.

Tetramer and Pentamer Analysis

T cells were labeled with anti-CD8-PE-Cy5, anti-CD4-APC, and anti-CD69-FITC antibodies and one of the following PE-conjugated iTag™ tetramers (Beckman Coulter) or pentamers (ProImmune): HCV NS5A (VLSDFKTWL; SEQ ID NO:3) HLA-A*0201, EBV (GLCTLVAML; SEQ ID NO:5) HLA-A*0201, HCV NS3 peptide (CINGVCWTV; SEQ ID NO:4) HLA-A*0201, HCV NS3 peptide (KLVALGINAV; SEQ ID NO:6) HLA-A*0201, and a negative control tetramer (multi-allelic). Approximately 100,000 cells were acquired using the FACSCalibur.

Analysis of Chimigen™ Protein Binding, Internalization and Processing by Confocal Microscopy

Binding, internalization and processing of the Chimigen™ Protein by immature DCs was studied using confocal microscopy. Immature DCs used in these studies were obtained by differentiating adherent PBMC derived monocytes for 2 days in the presence of GM-CSF and IL-4 in ATM V® media containing 2.5% donor matched serum. On day 2, immature DCs were transferred to chambered slides and incubated for an additional day before use. Day 3 was chosen as a compromise between cells having the appropriate cell surface receptors and morphology.

To study binding of Chimigen™ Protein to DC surfaces, cells were incubated with 5 Tg/mL Chimigen™ Protein or with buffer only as a negative control in PBSB at 4° C. for 1 hr. After 1 hr, cells were washed with PBSB and then labeled with biotinylated anti-mouse IgG1 antibody followed by streptvidin AlexaFluor® 546. PBSB washes were performed between each step. After labelling and washing, cells were fixed for 10 min. at 4° C. with 4% paraformaldehyde (made in PBSB). Slides were then mounted with SlowFade® Gold antifade reagent with 4′,6-diamidino-2-phenylindole, dihydrochloride (DAPI; Invitrogen) and cover slips were sealed onto the slides with nail polish.

Internalization of Chimigen™ protein (5 Tg/mL) by DCs was studied either by directly incubating the cells in media containing Chimigen™ Protein at 37° C. (7% CO₂) or by first labeling the surface receptors at 4° C. in PBSB, washing away the unbound protein, and then studying the uptake of the receptor bound protein over time (0 min., 15 min., 60 min. and 240 min.) at 37° C. (7% CO₂) in AIM V®/2.5% matched serum media. Cells incubated at 37° C. were washed with PBSB and then fixed and permeabilized for 10 min. with BD Biosciences Cytofix/Cytoperm™ solution. Cells were then washed and labeled (1 hr) with biotin anti mouse IgG1 in BD Biosciences Perm/Wash™ solution followed by labeling with streptavidin Alexa Fluor® 546. Co-labeling with other antibodies was performed as necessary. After the final washing of the cells, the slides were mounted as described above.

To confirm that the Chimigen™ Protein was endocytosed, pulse-chase experiments were performed. Immature DCs were pulsed with Chimigen™ Protein (5 Tg/mL) for 30 min. on ice. Cells were washed with PBSB and chased in AIM V®/2.5% matched serum media without Chimigen™ Protein and incubated at 37° C. (7% CO₂) for 15 min. Pulse-chased cells were washed with PBSB, fixed with 4% paraformaldehyde and labeled with MHC Class II antibody to label only the plasma membrane. To determine if the Chimigen™ Protein is present in endosomes, plasma membrane-labeled cells were then fixed and permeabilized for 10 min. with BD Bisosciences Cytofix/Cytoperm™ solution. After washing with BD Perm/Wash™, Chimigen™ Protein was detected with anti mouse IgG1 biotin/streptavidin, as described above.

For macropinocytosis studies, FITC Dextran (MW 70,000, anionic, lysine fixable, to Invitrogen) was used as a fluid phase marker at 5 mg/ml in AIM V®/2.5% matched serum medium either with or without the Chimigen™ Protein (5 Tg/mL).

To study receptor mediated endocytosis Alexa Fluor® 488 transferrin conjugate (Invitrogen) was used at 20 Tg/mL in PBSB containing Chimigen™ Protein (5 Tg/mL). Lactacystin (Sigma) was used both as a cysteine protease inhibitor and as a proteasome inhibitor (final conc. 5 Tg/mL).

### Evaluation of Immune Responses in In Vivo Animal Models

These studies use two inbred laboratory (mouse and rat) and one out-bred large animal (piglet) species. In particular, BALB/c mice (6-8 weeks old from Charles River Laboratories), Wistar rats (4-6 weeks old from Charles River Laboratories), and cross-bred piglets (4-6 weeks old from Prairie Swine Center, University of Saskatchewan) are used. The study determines immune responses and protective efficacy of Chimigen™ Protein.

Safety Evaluation

HCV Chimigen™ Proteins are administered either subcutaneously (s.c.) or intradermally (i.d.). The following protocol and doses is used for injections. Animals are immunized four times, on day 0, day 14, day 28, and day 42, every two weeks either s.c. or i.d. For mice s.c. and i.d. injections, a dose of 0.1 Tg, 1 ug or 10 Tg/mouse is used. The doses for the immunization of rats will be 0.15 Tg, 1.5 Tg or 15 Tg/rat and for piglets are 0.2 Tg, 2 Tg or 20 Tg/piglet.

Blood samples are collected pre-immunization (day −1) and 7 days after each injection (day 7, 21, 35, 49) for analysis of the quantity of specific antibodies as well as IgG1/IgG2a ratios by ELISA techniques.

Animals are sacrificed two weeks after final immunization. The safety profile of HCV Chimigen™ Proteins are evaluated by physical examination of the animals at least three times per week after immunization. This includes body weight and adverse event observation. For systemic toxicology, blood samples collected at regular intervals are used to monitor changes in serum chemistries, including aspartate aminotransferase (AST) and alanine aminotransferase (ALT) levels. In addition, at the end of the experiments, tissues collected from spleen, liver, kidney, heart, lung, muscle, and brain at the time of necropsy are fixed in 10% buffered formalin and embedded in paraffin for future analysis of potential pathological changes. Age-matched animals are used as controls.

Immune responses to Chimigen™ Proteins

Chimigen™ Proteins are predicted to induce strong cellular and humoral immune responses. Animal trials are performed to determine host immune responses to HCV Chimigen™ Caccines in piglets. Core, NS5A, NS3, and Multi-antigen Chimigen™ proteins are used for these studies. In the first round, immune responses to HCV Chimigen™ Proteins are evaluated. The proteins are given s.c. and i.d., as described above.

Splenocytes of mice or rats, and peripheral blood mononuclear cells (PBMCs) from piglets are used to determine the quantity and quality of immune responses to HCV Chimigen™ proteins following s.c. and i.d. routes of administration, as described below. These trials allow us to determine which of the Chimigen™ Vaccines and the routes of administration will induce the strongest immune response. The target-directed delivery of the HCV Chimigen™ proteins could elicit a potent Th1-biased immune response in addition to strong humoral response. Studies from VIDO on HCV vaccines have demonstrated that priming with DNA vaccines followed by protein boosting can induce strong and Th1/Th2-balanced immune responses [Yu et al. (2004) J. Gen. Virol. 85: 533-1543].

Evaluation of Immune Responses

i) Antibody responses. The presence of HCV antigen-specific antibodies is determined by ELISA to test total IgG as well as IgG1 and IgG2a antibody levels. The IgG levels demonstrate the quantity of the immune responses, while the relative levels of IgG1 and IgG2a demonstrate the quality (Th1 or Th2) of the immune responses. These experiments are performed using established protocols.

ii) Lymphocyte proliferation assays. Splenocytes of mice or rats, peripheral blood mononuclear cells (PBMCs) from piglets are stimulated with HCV antigens in vitro. Proliferative responses are measured by [methyl-³H] thymidine incorporation into the DNA of dividing cells.

iii) Cytokine ELISPOT assays. To further confirm the quality of the immune responses, the number of interferon-γ and interleukin-4 secreting cells in splenocytes (mouse and rat) or PBMCs (piglet) are determined in ELISPOT assays after stimulation with HCV antigens as per our established protocol.

Protective Anti-Viral Immunity Induced by HCV Chimigen™ Proteins

HCV has a very narrow host range. It replicates only in humans and chimpanzees. Challenge of vaccinated animals with live HCV is not practical, as chimpanzees are expensive and limited in supply. However, challenging with recombinant vaccinia virus encoding an HCV antigen after vaccination is an alternative model for evaluation of protective ability induced by HCV prophylactic vaccines in animal models. Chimigen™ Proteins, individually or as combinations, are used to immunize the animals. To evaluate the protective immunity induced following the vaccinations, animals are challenged intraperitoneally by recombinant vaccinia viruses encoding the same HCV antigen as relevant Chimigen3 Proteins two weeks after the completion of the scheduled vaccination using a pre-determined strategy. The challenge doses will be 1×10⁷ plaque-forming units (PFUs) for mice, 2×10⁷ PFUs for rats, and 1×10⁹ PFUs for piglets. Five days later, animals will be sacrificed and vaccinia virus titers will be determined by plaque assays as per established protocol.

Identification of the Most Suitable Candidate(s) for HCV Therapeutic and Prophylactic Vaccines

Therapeutic vaccines are based on non-structural proteins of the HCV virus (e.g. NS5A, NS3), whereas the prophylactic vaccines are based on structural proteins (e.g. E1, E2) as well as non-structural proteins. A combination of one or more of the Chimigen™ Vaccines is used in both the ex vivo DC/T cell antigen presentation assays and in the animal models and the immunological outcome is evaluated.

The immunization protocols as well as the route of administration of the Chimigen™ Vaccines for therapeutic and prophylactic uses are currently being studied. The immune responses are evaluated by measuring antibody levels, lymphocyte proliferation and cytokine production. Protective anti-viral immunity induced by the prophylactic HCV Chimigen™ Vaccine candidates is also be evaluated in challenge experiments, as described above.

### Example 2

### Results with NS5A Chimigen3 Protein

NS5A Chimigen™ Protein has been Purified and Characterized

Purified NS5A Chimigen™ protein migrated by SDS-8% PAGE as a band of ˜105 kDA, although the predicted molecular weight of the protein is ˜81 KDa. The discrepancy between the observed and predicted molecular weights may result in part due to the high proline content (˜11%), glycosylation and other possible post-translational modification of the protein. The purified protein was detected with antibodies against mouse IgG1 Fc, 6×His tag and NS5A. MS/MS ID (Mass Spectrometry) analysis on the purified protein (band cut from gel) gave significant hits for NS5A, mouse IgG1 heavy chain and HCV polyprotein indicating it was indeed the NS5A Chimigen™ Protein. Purified NS5A Chimigen™ Protein was separated on a 8% SDS gel and stained for glycosylation using the Pro-Q® Emerald 300 Glycoprotein Gel and Blot Stain Kit and detected by UV illumination. This procedure showed glycosylation of the purified NS5A Chimigen™ Protein.

### NS5A Chimigen™ Protein Binds to Immature DCs

NS5A Chimigen™ Protein was examined for its ability to bind to immature DCs. The cells were incubated in the presence and absence of various concentrations of NS5A Chimigen™ Protein for 1 hr at 4° C. The bound vaccine was detected with biotinylated anti-mouse IgG1 mAb and SA-PE-Cy5. The percentage of cells binding the vaccine (% positive cells) and the relative amount of bound protein (MFI) was determined by flow cytometry (FIG. 15). With NS5A Chimigen™ Protein at 4-50 μg/mL, most DCs were positive for binding, and there was a dose-dependent increase in the amount of bound protein (FIGS. 15 and 16). Binding of the protein was not much greater at 20 μg/mL compared with 50 μg/mL indicating that the binding to immature DCs was saturable. The high MFI of binding observed suggests that NS5A Chimigen™ Protein binds very effectively and at high levels to immature DCs. That the binding at 4° C. was saturable suggests that the process is receptor-mediated. The binding was also very rapid with bound protein detected after 5 min of incubation at 4° C. with a MFI of binding approximately half of that observed after 1 hr incubation (data not shown)

### NS5A Chimigen™ Protein Binds to Specific Receptors on Immature DC

By virtue of the Fc fragment it contains, the NS5A Chimigen™ Protein is predicted to bind via its TBD region to CD32 (FORM on immature DCs. In addition, due to its mannose glycosylation, NS5A Chimigen™ Protein is predicted to bind to C-type lectin receptors such as CD206 (MMR). To determine the specificity of binding of the vaccine candidate, immature DCs were incubated with NS5A Chimigen™ Protein in the presence of blocking anti-CD32 and/or anti-CD206 mAbs.

Immature DCs were incubated with buffer control, or 5 μg/ml of isotype control mAb, anti-CD32 mAb, anti-CD206 mAb, or both anti-CD32 and anti-CD206 for 1 hr at 4° C. before incubation with NS5A Chimigen™ Protein for 1 hr at 4° C. Bound NS5A Chimigen™ Protein was detected with biotinylated anti-6×His mAb followed by SA-PE-Cy5. Isotype control mAbs (murine IgG1 and IgG2b) did not inhibit binding compared with buffer control. However in comparison with buffer control, both anti-CD32 and anti-CD206 inhibited binding by approximately 60% and 40%, respectively.

The addition of both mAbs further inhibited NS5A Chimigen™ Protein binding resulting in an 80% inhibition. These data indicate a role for both CD32 and CD206 in the binding of NS5A Chimigen™ Protein. By confocal microscopy, the cells incubated with NS5A Chimigen™ Protein at 4° C. showed an intense labeling on their surface compared with buffer only controls indicating the Chimigen™ Protein bound to the cell surface.

The internalization of the NS5A Chimigen™ Protein by immature DCs was evaluated at 37° C. Immature DCs incubated with Chimigen™ Protein at 37° C. for 1 hr showed a punctuate labeling pattern, often in the vicinity of the nucleus, suggesting the Chimigen™ Protein was internalized and there was very little, if any, surface labeling.

DCs are capable of antigen uptake via several routes which include phagocytosis, macropinocytosis, clathrin-mediated endocytosis and non-clathrin/caveolae endocytosis. Macropinocytosis is reported to be a constitutive process in immature DCs (Trombetta and Mellman 2005). The ability of immature DCs to internalize NS5A Chimigen™ Protein by macropinocytosis at 37° C. was evaluated using the macropinocytosis marker FITC dextran [Hewlett et al. (1994) J. Cell Biology 124:689-703]. After incubating immature DCs for 15 min and 60 min with the Chimigen™ Protein and FITC dextran, vesicle-like structures were observed which contained both protein and FITC dextran, indicating that at 37° C. the Chimigen™ Protein may be taken up by macropinocytosis. It should also be noted that FITC Dextran may bind to macrophage mannose receptors (CD206) and thus some of the endosomes containing both Chimigen™ Protein and FITC Dextran may have arisen by receptor-mediated endocytosis.

The role of receptor mediated endocytosis in the uptake of Chimigen™ Protein was studied by pulse-chase experiments. Immature DCs were pulsed with fluorescent labeled Chimigen™ Protein at 4° C., washed, and incubated with for 15 min at 37° C. The cells were fixed, permeabilized and labeled with antibodies to detect the Chimigen™ Protein as well as the transferrin receptor. The transferrin receptor is taken up by receptor mediated endocytosis and then recycled back to the plasma membrane from early endosomes. At 4° C., the NS5A Chimigen™ Protein bound to the surface of the cells while the transferrin receptor was present predominantly within the cells. On switching to 37° C., endosomes were observed to form, some of which contained both the Chimigen Protein™ and transferrin receptors. The large number of pre-existing intracellular transferrin receptors at the start of the experiment (4° C.) is probably responsible for many transferrin containing endosomes not co-localized with the Chimigen™ Protein.

The uptake of the NS5A Chimigen™ Protein by DCs and its co-localization with transferrin, by co-labeling with an antibody against the transferrin receptor also was evaluated using confocal microscopy. Transferrin binds to transferrin receptor and is known to be internalized by receptor-mediated processes. This analysis showed co-localization of the two molecules, thereby indicating that NS5A Chimigen™ Protein is taken up by receptor-mediated endocytosis.

In an attempt to increase the overlap between Chimigen™ Protein and transferrin receptor signals, cells were incubated with Alexa Fluor 488 conjugated to human transferrin so as to indirectly detect only recently endocytosed transferrin receptors rather than all transferrin receptors in the cell. Immature DCs were surface labeled at 4° C. with a mixture of Chimigen™ Protein and Alex Fluor 488 conjugated to transferrin, Few Alexa Fluor 488 transferrin positive endosomes were observed, but when present they contained the NS5A Chimigen™ Protein indicating that the Chimigen™ Protein is indeed taken up by receptor mediated endocytosis. These results show that the Chimigen3 Protein is predominantly internalized by receptor-mediated endocytosis.

The processing of the NS5A Chimigen™ Protein by immature DCs was studied. Since the vaccine is designed, inter alfa, to treat chronic infections, activation of CD8+ cells and antigen cross presentation via MHC Class I receptors is required. Two different processing routes have been proposed for antigen cross presentation [Lizée et al. (2005) Trends Immunol. 26(3):14′-149]. The first involves processing of antigens taken up by phagocytosis, while the second involves processing of antigens taken up be other routes of endocytosis such as receptor mediated endocytosis. In the second route proteins are taken up into early endosomes and the targeted to late endosomes where they are broken down by cathepsins and then loaded onto MHC Class I receptors. Thus, experiments were performed to determine if the NS5A Chimigen™ Protein could be detected in late endosomes and also to determine if it co-localizes with MHC Class I receptors in such structures. Cells that had been pulsed with NS5A Chimigen™ Protein at 4° C. and then chased at 37° C. were co-labeled to detect both the Chimigen™ Protein and LAMP1 (a marker of late endosomes/lysosomes). At 4 hr and 24 hr, in a few cells, overlap was observed between the NS5A chimigen protein and LAMP1 indicating that the NS5A chimigen protein was in late endosomes/lysosomes. In another co-labeling experiment, the NS5A Chimigen™ Protein was found to be present in similar structures with MHC Class I molecules, thereby indicating that the Chimigen™ Protein is processed for presentation via MHC Class I molecules.

Proteasomes are believed to be involved in the breakdown of antigens for cross presentation via the phagolysosome pathway, Cells were treated with the cell-permeable proteasome inhibitor Lactacystin at a concentration of 5 μg/mL. Lactacystin recently has been shown to be less specific than previously thought at inhibiting the lysosomal protease Cathepsin A [Kozlowski et al. (2001) Tumour Biol. 22(4):211-215]. If processing of NS5A Chimigen™ Protein involved proteasomes as in the phagolysosome pathway, then one would expect a partial accumulation of incompletely degraded peptides in the cytosol and such an accumulation would not be expected for processing in the late endosome [Lizée et al. (2005) supra]. Pulsed cells treated with 5 ug/mL of lactacystin (pulse and chase) after a 4 hr chase showed a larger number of endosomes containing the NS5A Chimigen™ Protein. In addition, the endosomes appeared to contain more of the protein than those of control cells. An increase in cytoplasmic NS5A Chimigen™ protein could not be detected with a monoclonal antibody against mouse IgG1. These data again point to the receptor-mediated endocytosis rather than the phagolysosome pathway being the mechanism whereby Chimigen3 Proteins are internalized in DC.

NS5A Chimigen™ Protein Presentation by DCs Results in both CD8+ and CD4+ T Cell Activation and Proliferation

The functional immune response to NS5A Chimigen™ Protein was assessed by ex vivo APAs. This assay can be used to measure various parameters of a functional T cell immune response after stimulation of T cells with antigen-loaded DCs. The assay consists of first generating immature DCs from PBMC-derived monocytes by the addition of IL-4 and GM-CSF. The immature DCs are then incubated with vaccine candidate, carrier buffer (negative control), or tetanus toxoid (TT) (positive control). The DCs are then treated with cytokines to undergo maturation, washed, and incubated with autologous naïve T cells. For measuring cytokine production, the presence of cytotoxic granule components, and the generation of NS5A-specific T cells, the T cells are stimulated an additional two times allowing for the expansion of Chimigen™ Protein specific T cells. Activation was assessed by measuring the early T cell activation marker CD69, and proliferation was measured by tracking the fluorescence of CFSE labeled T cells. Both CD69 expression and CFSE fluorescence were evaluated after 4 and 7 days of culture with antigen-loaded DCs.

A preliminary analysis had indicated that the concentration of DCs and T cells in the culture were important parameters in the determination of T cell immune response. Thus an APA was designed such that six different T cell:DC ratios were assessed. Two sets of DC concentrations were used, a high concentration of 5×10⁴ DCs/well and a low concentration of 1×10⁴ DCs/well. After 48 hr of culture, the immature DCs were incubated with buffer (negative control), two different preparations of NS5A Chimigen™ Protein (5AC) at 5 μg/mL, TT (positive control), or PBS. The DCs were then cultured for 8 hr and matured by the addition of poly IC, IL-1, IL-6, TNF-α, IFN-α, and IFN-γ. After culture overnight (16 hr) DCs from the PBS control group were washed and examined for the expression of various mature DC markers. Both high and low concentration DCs expressed high levels of HLA-ABC (MHC class I), HLA-DR (MHC class II), CD86, CD80, and CD83 (FIG. 17). In general, the high concentration DCs expressed slightly higher levels of DC maturation markers.

Autologous T cells were isolated by a negative selection procedure and labeled with CFSE for the determination of cell division. To 100 μl/well of DCs in a 96-well plate, 100 μl/well of T cells were added for concentrations per well of: 20×10⁴, 5×10⁴, or 2×10⁴. For the high DC concentration wells (5×10⁴ DC/well) the T cell to DC per well ratio combinations were: 20×10⁴:5×10⁴ (4:1), 5×10⁴:5×10⁴ (1:1), and 2×10⁴:5×10⁴ (0.4:1). For the low DC concentration wells (1×10⁴ DC/well), the T cell to DC ratio combinations per well were: 20×10⁴:1×10⁴ (20:1), 5×10⁴:1×10⁴ (5:1), and 2×10⁴:1×10⁴ (2:1). T cells were added to the DC in the absence of any exogenous cytokines. As a control, at day 3 of culture PHA at 1 μg/mL was added to the T cells loaded onto the PBS treated DC group.

Following 4 days of culture, half of the cell culture (1000 was harvested for analysis of activation and proliferation. To the remaining half of the cell culture, 1000 of fresh AIM V®/2.5% matched sera was added and the cells cultured for an additional 3 days. The expression of CD69 on the T cells following 4 days of culture is shown in FIGS. 18A-C. FIG. 18A shows the percentage of CD3+ cells expressing CD69 for the different T cell:DC ratios. The majority of PHA treated cells expressed CD69 regardless of the T cell:DC ratio. CD69 was also detected in T cells cultured with the high DC concentration but was barely detected in T cells cultured with the low DC concentration. Compared with buffer control, antigen stimulated T cells expressed a higher level of CD69. NS5A Chimigen™ Protein-loaded DCs induced a higher percentage of CD69 expressing CD8+ T cells than CD4+ T cells at day 4 (FIGS. 18B and C). The percentage of CD69 expression of CD8+ T cells was equivalent or greater for the Chimigen3 Protein compared with the recall antigen TT. This indicated that the NS5A Chimigen™ Protein is a strong activator of naïve CD8+ T cells

The percentage of cells that had undergone at least one division) (CFSE^{lo}) after four days of culture is shown in FIGS. 19A-C. T cells treated with PHA 24 hr earlier had begun to divide (FIG. 19A). CD8+ and CD4+ T cells treated with TT-loaded DCs undergo detectable proliferation after 4 days of culture but this was only evident at the high DC concentration (FIGS. 19B and C). There was little detection of T cell proliferation in the Chimigen™ Protein-treated groups at day 4. Thus naïve T cells were activated by NS5A Chimigen™ Protein-loaded DCs on day 4 of culture as evidenced by expression of CD69 but these T cells have not yet divided or had divided undectably by the assay used.

Following 7 days of culture, cells were harvested for analysis of activation and proliferation. The expression of CD69 on the T cells following 7 days of culture is shown in FIGS. 20A-C FIG. 20A shows the percentage of CD3+ cells expressing CD69 for the different T cell:DC ratios. There was a marked increase in CD69 expression of the T cells treated with PHA. However, the percentage of cells expressing CD69 has decreased from that observed at day 4 consistent with what is expected from a PHA response; rapid induction of CD69 followed by a decrease in expression with time. For Chimigen3 Protein-stimulated T cells, CD69 was detected at levels over 5% in T cells cultured with the high DC concentration but was barely detected in T cells cultured with the low DC concentration. Thus, the low DC concentration (1×10⁴ DC/well) was not sufficient for antigen-specific T cell activation. Compared with buffer control, a greater number of Chimigen3 Protein-stimulated T cells expressed CD69 for the 5×10⁴ T cell and 2×10⁴ T cell:5×10⁴ DC ratios. The expression of CD69 was reduced for the recall TT response at day 7. In contrast to d4 T cells, NS5A Chimigen™ Protein-loaded DCs induced a higher percentage of CD69 expressing CD4+ T cells than CD8+ T cells at day 7 (FIGS. 20B and C). For the higher DC concentration (5×10⁴/well) the percentage of CD69 expression of CD8+ and CD4+ T cells was equivalent or greater for the Chimigen™ Protein compared with buffer or the recall antigen TT Thus the NS5A Chimigen™ activates naïve CD8+ T cells initially, followed by CD4+ T cells

The percentage of cells that have undergone at least one division (CFSE^{lo}) after seven days of culture are shown in FIGS. 21A-C. The results show that essentially every T cell treated with PHA has divided (FIG. 21A). DCs loaded with Chimigen3 Protein or TT resulted in marked T cell proliferation after 7 days of culture but this was most evident at the high DC concentration. Only the high DC concentration wells induced marked CD8+ T cell proliferation as a result of antigen loading (FIG. 21B). However, the low concentration of DCs loaded with antigen was sufficient to induce CD4+ T cell proliferation (FIG. 21C). Notably, at the high DC concentration, the Chimigen3 Protein-loaded DCs induced T cell proliferation to levels comparable to TT-loaded DCs

Another measure of T cell proliferation is the relative proportion of blast T cells in the T cell population. Blast T cells are defined as those cells possessing a higher FSC (forward light scatter) and SSC (sidelight scatter) then the resting lymphocytes in the lymphocyte gate as assessed by flow cytometry. The percentage of T cell blasts in the cultures is shown in FIG. 22. These results correlate very well with the percentage of cells undergoing division as shown in FIG. 21A. Therefore, the assessment of T cell blasts in a population can be used as an alternative to the CFSE assay. Overall, these findings indicate that the NS5A Chimigen™ Protein was quite efficient at inducing a primary T cell response as measured by T cell activation and proliferation.

### NS5A Chimigen™ Protein Presentation by DCs Results in the Generation of CD8+ and CD4+ T Cells Producing IFN-γ and TNF-α

The functional immune response to NS5A Chimigen™ vaccine was assessed by a three stimulation ex vivo APA. Immature DCs at either 4×10⁴ DCs/well (high concentration) or at 2×10⁴ DCs/well (low concentration) were loaded with control carrier buffer, PBS, TT (positive control), or NS5A Chimigen™ Protein. DCs were then matured and their phenotype evaluated. The DCs maturation was established using the high level expressions of MHC class I, MHC class II, CD86, CD80, and CD83 (FIG. 23). Autologous T cells were incubated with the matured antigen-loaded DCs at a ratio of 20×10⁴ T cells/well:4×10⁴ DCs/well or 4×10⁴ T cells/well:2×10⁴ DCs/well. The T cells were stimulated three times and T cell function evaluated 6 hr following the third stimulation by detection of the intracellular levels of the Th1 cytokines IFN-γ and TNF-α. In addition the level of blast T cells was also assessed.

FIG. 24 shows the percentage of blast T cells at the high and low DC concentrations. The 2:1 T cell:DC ratio resulted in a lower background (buffer) T cell proliferative response compared with the 5:1 ratio. As a result with the 2:1 ratio there was a more marked difference between buffer and antigen-induced T cell proliferative response.

The IFN-γ response was measured at the 5:1 and 2:1 T cell:DC ratios. The data is shown as the responses of each well of the group and as an average of the three wells with the standard deviation of the mean (FIG. 25). A comparison of the T cell IFN-γ response showed a marked difference between the 5:1 and 2:1 T cell:DC ratios. With the higher DC concentration there was no evidence of a Chimigen 3-induced IFN-γ response over that of control buffer. However with the lower DC concentration, very few T cells cultured with control buffer-loaded DCs produced IFN-γ whereas a high percentage of T cells cultured with Chimigen3 Protein-loaded DCs produced IFN-γ. There were more IFN-γ producing cells in the T cells stimulated with DCs that had been loaded with 5 μg/mL compared with 2.5 ug/mL of NS5A Chimigen™ Protein. The percentage of T cells expressing IFN-γ in the CD8+ and CD4+ population was also measured (FIGS. 26 and 27). The low DC concentration groups showed a high, percentage of CD8+ T cells expressing IFN-γ as a result of stimulation with Chimigen3-DCs. The percentage of CD8+ T cells expressing IFN-γ was higher for T cells stimulated with Chimigen3 Protein-loaded DCs compared with TT-loaded DCs (FIG. 26). Similarly, there was also a high percentage of CD4+ T cells that expressed IFN-γ upon stimulation with NS5A Chimigen™ Protein compared with control buffer (FIG. 27). The percentage of CD4+ T cells expressing IFN-K was comparable for T cells stimulated with Chimigen3-Protein-loaded DCs compared with TT-loaded DCs (FIG. 27). These results indicate that NS5A Chimigen™ Protein induces a marked IFN-γ response in both CD8+ and CD4+ T cell populations and suggests that the molecule is processed by the DCs in both the MHC class I and class U pathways.

FIG. 28 shows the percentage of T cells that have produced TNF-α as a result of a 6 hr stimulation with antigen-loaded mature DCs. These results are similar to the IFN-γ results. Although there was an increase in the percentage of cells producing TNF-α as a result of antigen stimulation of the T cells with the high DC concentration (5:1 ratio), there was an even greater difference with the low DC concentration (2:1 ratio). A higher percentage of T cells produced TNF-α when stimulated by DCs loaded with 5 μg/mL of Chimigen3 Protein compared with 2.5 μg/mL of protein. The TNF-α response was greater for the NS5A Chimigen™ Protein compared with TT. Stimulation with TT-loaded DCs resulted in a higher percentage of CD4+ T cells expressing TNF-α compared with CD8+ T cells (FIG. 29). However, NS5A Chimigen™ Protein-loaded DCs induced a similar degree of TNF-α production in both CD8+ and CD4+ T cell populations (FIG. 29).

NS5A Chimigen™ Antigen Presentation by DCs Results in the Generation of CD8+ T Cells Expressing grB and pfn+ and CD4+ T Cells 6 hr Post 3^{rd} stimulation

The ability of T cells to produce the cytotoxic granular proteins grB and pfn was also assessed by ex vivo antigen presentation assays. Immature DCs were loaded with control buffer, with TT (positive control), or varying concentrations of NS5A Chimigen™ Protein and upon maturation were incubated with autologous T cells. The expression of grB can be detected in different ways, including enzymatic assays and by specific antibodies [Ewen et al. (2003) J. Immuno]. Meth. 276:89-101; Spaeny-Dekking et al (1998) J. Immunol. 160:3610; Hamann et al. (1997) J. Exp. Med. 186:1407]. GrB and pfn expression were detected by intracellular staining using an anti-grB and anti-pfn mAbs, respectively. FIG. 30 shows the percentage of CD8+ T cells that express grB and pfn following three stimulations with antigen-loaded mature DCs. NS5A Chimigen™Protein-loaded DCs induced an increase in grB and pfn expression in. CD8+ T cells compared to the no antigen control. These results indicate that NS5A Chimigen™ Protein induces the expression of grB and pfn in CD8+ T cells and this suggests that this protein is processed by the DCs in the class I pathways for the effective presentation to T cells which results in their differentiation from naïve CD8+ T cells to cytotoxic T lymphocytes (CTLs).

### NS5A Chimigen™ Antigen Presentation by Mature DCs Results in the Generation and Maintained Activation of CD8+ and CD4+ T Cells

T cells were stimulated with antigen-loaded DCs three times in an APA. After 6 days of culture following the third stimulation the T cells were harvested and investigated by FFC for the percentage of blast cells as a measure of proliferation and for the expression of the activation marker CD69. In addition as a means to estimate absolute numbers of T cells recovered from culture, the number of gated cells falling in the lymphocyte gate (R1 gate) based on FSC and SSC flow cytometric analysis was determined.

There was a marked difference in the recovery of T cells from TT and Chimigen™ Protein stimulated cells compared with buffer control (FIG. 31). TT stimulated cells gave a higher T cell recovery than the Chimigen3 Protein stimulated cells. However the TT response is a recall response and thus the starting population of T cells specifically responsive to TT would be expected to be higher than that of the starting population of naïve T cells specific for NS5A. Notably, on assessment of the blast cell population, the percentage of blast cells/proliferating cells was actually higher in the NS5A Chimigen3 Protein cultures compared to the TT cultures. There were very few blast cells/proliferating cells in the buffer control cultures. The percentage of activated CD8+ and CD4+ T cells as assessed by CD69 expression is shown in FIG. 32. There was a high percentage of both CD4+ and CD8+ T cells expressing CD69 in T cells stimulated with Chimigen3 Protein-loaded DCs compared with buffer control. These results show that the stimulation with the Chimigen3 Protein results in marked T cell activation and proliferation that is evident even six days following the third stimulation (day 20 of T cell culture). The Chimigen3 Protein is therefore very effective in the activation and expansion of both CD8+ and CD4+ T cells.

### NS5A Chimigen™ Protein Presentation by Mature DCs Induces the Generation of NS5A-Specific CD8+ T Cells

To evaluate the antigen-specificity of the immune response to NS5A Chimigen™ Protein, the percentage of T cells specific to an immunodominant NS5A epitope in the context of HLA-A2 was quantitated. This was determined by labeling T cells with an NS5A peptide/HLA-A2 pentamer conjugated to PE. Naïve T cells were stimulated three times with DCs loaded with different concentrations of NS5A Chimigen™ Protein and compared to the respective control DCs loaded without antigen (buffer) in an APA. T cells were harvested six days after the third stimulation and NS5A-specific T cells or EBV-specific T cells (control) detected by tetramer labeling and FFC.

The percentages of negative tetramer labeling (negative control) and EBV tetramer labeling (positive control) CD8+ and CD4+ T cells are shown in FIG. 33. One well of the three tested was positive for EBV tetramer labeling (positive tetramer) in the CD8+ T cell population. As the T cells assessed were from the buffer control treated wells, it would be expected that the number of EBV tetramer-labeled T cells would be relatively low. The percentage of CD8+ T cells labeling with an NS5A pentamer following the APA is shown in FIG. 34. Loading DCs with NS5A Chimigen™ Protein resulted in the generation of T cells with specificity to the NS5A epitope VLSDFKTWL (SEQ ID NO:3). The marked expansion of CD8+ T cells with this specificity was apparent in two wells of the high DC concentration wells and three wells of the low DC concentration wells. Thus the NS5A Chimigen3 Protein is able to induce the generation of T cells specific to this NS5A immunodominant epitope and it is likely that T cells are present with specificities to other NS5A epitopes.

### Example 3

### Results with NS3 Chimigen3 Protein

NS3 Chimigen™ Protein has been Purified and Characterized

NS3 Chimigen™ Protein expressed in Sf9 cells was purified by Ni-NTA affinity chromatography followed by cation exchange chromatography. Purified samples were analyzed using 10% SDS-PAGE gels. After electrophoresis, gels were transferred to nitrocellulose for Western blotting. The SDS-PAGE gel was stained with PageBlue and Western blots were developed with antibodies specific for different components of the NS3 Chimigen™ Protein. The purified protein appeared as a doublet at approximately 110 KDa and 120 KDa. Both species were detected by antibodies against the N-terminus (anti-6×His), TBD (anti-Fe) and NS3 (polyclonal anti-NS3), which indicated that the purified protein was intact:

A qualitative assessment of glycosylation of purified NS3 Chimigen™ Protein was performed using the Pro-QS® Emerald 300 Glycoprotein Gel and Blot Stain Kit. After electrophoresis of purified protein on an 8% SDS polyacrylamide gel, the gel was stained using the manufacturer's protocol and scanned under illumination with UV. Since purified protein is a doublet, the difference in molecular weight is presumed to be due to the different levels of glycosylation.

### NS3 Chimigen™ Protein Binds to Immature DCs

NS3 Chimigen™ Protein was examined for its ability to bind to immature DCs. The cells were incubated in the presence and absence of various concentrations of NS3. Chimigen™ Protein for 1 hr at 4° C. The bound protein was detected with biotinylated anti-mouse IgG1 mAb and SA-PE-Cy5. The percentage of cells binding the Chimigen3 protein (% positive cells) and the relative amount of bound protein (MFI) was determined by FFC. With NS3 Chimigen™ Protein at 4-55 μg/mL, most DCs were positive for binding, and there was a dose-dependent increase in the amount of bound protein (FIGS. 35 and 36). Binding of the protein was not much greater at 22 μg/mL compared with 55 μg/mL indicating that the binding to immature DCs was saturable. The high MFI of binding observed indicated that NS3 Chimigen™ Protein binds very effectively and at high levels to immature DCs. The binding at 4° C. was saturable, indicating that it is receptor-mediated. The binding was also very rapid with bound protein detected after 5 min of incubation at 4° C. with a MFI of binding approximately half of that observed after a 60 min incubation (data not shown).

### NS3 Chimigen™ Protein Binds to Specific Receptors on Immature DCs

By virtue of the presence of Fc fragment, NS3 Chimigen™ Protein is predicted to bind via its TBD region to CD32 (FcγRII) on immature DCs. In addition, due to its mannose glycosylation, NS3 Chimigen™ Protein is predicted to bind to C-type lectin receptors such as CD206 (MMR). To determine the specificity of binding of the protein, immature DCs were incubated with NS3 Chimigen™ Protein in the presence of blocking anti-CD32 and/or anti-CD206 mAbs:

Immature DCs were incubated with buffer control, or 5 μg/mL of isotype control mAb, anti-CD32 mAb, anti-CD206 mAb, or both anti-CD32 and anti-CD206 for 1 hr at 4° C. before incubation with NS3 Chimigen™ Protein for 1 hr at 4° C. Bound NS3 Chimigen™ Protein was detected with biotinylated anti-6×His mAb followed by SA-PE-Cy5. Isotype control mAbs (murine IgG1 and IgG2b) did not inhibit binding compared with buffer control. However in comparison with buffer control, both anti-CD32 and anti-CD206 inhibited binding by approximately 70% and 60%, respectively (FIG. 36). The addition of both blocking mAbs further inhibited NS3 Chimigen™ Protein binding, resulting in a 90% inhibition (FIG. 36). Thus, the data indicates a role for both CD32 and CD206 in the binding of the NS3 Chimigen™ Protein binding to immature DCs.

The binding of NS3 Chimigen™ Protein was visualized by confocal microscopy. Immature DCs were incubated with NS3 Chimigen™ Protein at 4° C. Strong labeling on the cell surface compared with buffer only controls indicated that the Chimigen™ Protein bound to the surface of the cells, possibly to receptors.

To investigate internalization, immature DCs were incubated with the Chimigen™ Protein at 37° C. for 1 hr. The cells showed little if any surface labeling (plasma membrane outlines) but instead showed a punctuate labeling pattern often in the vicinity of the nucleus, indicating that the Chimigen™ Protein was internalized.

NS3 Chimigen™ Protein Presentation by DCs Results in both CD8+ and CD4+ T Cell Activation and Proliferation

The functional immune response to NS3A Chimigen™ Protein was assessed by ex vivo antigen presentation assays (APAs). This assay can be used to measure various parameters of a functional T cell immune response after stimulation of T cells with antigen-loaded DCs. The assay consists of first generating immature DCs from PBMC-derived monocytes by the addition of IL-4 and GM-CSF. The immature DCs are then incubated with vaccine candidate, carrier buffer (negative control), or TT (positive control). Subsequently DCs are treated with cytokines to undergo maturation, washed, and incubated with autologous naïve T cells. For measuring cytokine production, the presence of cytotoxic granule components, and the generation of NS3-specific T cells, the T cells are stimulated an additional two times allowing for the expansion of Chimigen™ Protein specific T cells. However a single stimulation would be expected to initiate expansion from a naïve T cell population. Activation was assessed by measuring the early T cell activation marker CD69, and proliferation was measured by tracking the fluorescence of CFSE labeled T cells. Both CD69 expression and CFSE fluorescence were evaluated after 4 and 7 days of culture with antigen-loaded DCs.

Preliminary analysis had indicated that the concentration of DCs and T cells in the culture were important parameters in the determination of T cell immune response. Thus the APA was designed such that six different T cell: DC concentrations were assessed. Two sets of DC concentrations were used, a high concentration of 5×10⁴ DCs/well and a low concentration of 1×10⁴ DCs/well. After 48 hr of culture, the immature DCs were incubated with buffer (negative control), NS3 Chimigen™ Protein (3C) at 5 μg/ml, TT (positive control), or PBS. The DCs were then cultured for 8 hr and matured by the addition of poly IC, IL-1, IL-6, TNF-α, IFN-α, and IFN-γ. After culture overnight (16 hr) DCs from the PBS control group were washed and examined for the expression of various mature DC markers. Both high and low concentration DCs expressed high levels of HLA-ABC (MHC class I), HLA-DR (MHC class II), CD86, CD80, and CD83.

Autologous T cells were isolated by a negative selection procedure and labeled with CFSE for determination of cell division. To 100 μl/well of DCs in a 96-well plate, 100 μl/well of T cells were added for concentrations per well of: 20×10⁴, 5×10⁴, or 2×10⁴. For the high DC concentration wells (5×10⁴ DC/well) the T cell to DC per well ratio combinations were: 20×10⁴:5×10⁴ (4:1), 5×10⁴:5×10⁴ (1:1), and 2×10⁴:5×10⁴ (0.4:1). For the low DC concentration wells (5×10⁴ DC/well), the T cell to DC ratio combinations per well were: 20×10⁴:1×10⁴ (20:1), 5×10⁴:1×10⁴ (5:1), and 2×10⁴:1×10⁴ (2:1). T cells were added to the DCs in the absence of any exogenous cytokines. As a control, at day 3 of culture PHA at 1 μg/mL was added to the T cells loaded onto the PBS treated DC group.

Following 4 days of culture, half of the cell culture (100 μl) was harvested for analysis of activation and proliferation. To the remaining half of the cell culture, 100 μl of fresh AIM V®/2.5% matched sera was added and the cells cultured for an additional 3 days. The expression of CD69 on the T cells at the 5×10⁴ T cells/well:5×10⁴ DCs/well ratio (1:1) after 4 and 7 days of culture is shown in FIG. 37. The majority of PHA treated cells expressed CD69 regardless of the T cell:DC ratio. CD69 was detected in T cells cultured with the high DC concentration but was barely detected in T cells cultured with the low DC concentration (data not shown). Compared with buffer control, antigen stimulated T cells expressed a higher level of CD69. NS3 Chimigen™ Protein-loaded DCs induced a higher percentage of CD69 expressing CD8+ T cells than CD4+ T cells at day 4. The percentage of cells that have undergone at least one division) (CFSE^{lo}) after four days of culture is shown in FIG. 38. The results indicate that the T cells treated with PHA 24 hr earlier had begun to divide. CD8+ and CD4+ T cells treated with TT-loaded DCs undergo detectable proliferation after 4 days of culture but this was only evident at the high DC concentration (data not shown). There was little detection of T cell proliferation in the vaccine candidate treated groups at day 4. Thus naïve T cells are activated by NS3 Chimigen™ Protein-loaded DCs on day 4 of culture as evidenced by expression of CD69 but these T cells have not yet divided.

Following 7 days of culture, cells were harvested for analysis of activation and proliferation. The expression of CD69 on the T cells following 7 days of culture is shown in FIG. 37. For Chimigen3 Protein stimulated T cells, CD69 was detected at levels over 5% in T cells cultured with the high DC concentration, but was barely detected in T cells cultured with the low DC concentration (data not shown). Thus the low DC concentration (1×10⁴ DC/well) was not sufficient for antigen-specific T cell activation. The expression of CD69 was reduced for the recall TT response at day 7. In contrast to d4 T cells, NS3 Chimigen™ Protein-loaded DCs induced a higher percentage of CD69 expressing CD4+ T cells than CD8+ T cells at day 7. The percentage of CD69 expression of CD8+ and CD4+ T cells at day 7 was greater for the Chimigen3 Protein compared with the recall antigen TT. Thus the Chimigen3 Protein initially activates naïve CD8+ T cells, followed by CD4+ T cells. The percentage of cells that have undergone at least one division) (CFSE^{lo}) after seven days of culture is shown in FIG. 38. DCs loaded with Chimigen3 Protein or TT resulted in marked CD8+ and CD4+ T cell proliferation after 7 days of culture and this was most evident at the high DC concentration (results not shown).

### NS3 Chimigen™ Protein Presentation by DCs Results in the Generation of CD8+ and CD4+ T Cells Producing IFN-γ and TNF-α

The functional immune response to NS3 Chimigen™ Protein was assessed by a three stimulation ex vivo APA. Immature DCs at either 4×10⁴ DCs/well (high concentration) or at 2×10⁴ DCs/well (low concentration) were loaded with control carrier buffer, PBS, TT (positive control), or NS3 Chimigen™ Protein. DCs were then matured and their phenotype evaluated. The DCs were assessed as mature as they expressed high levels of MHC class I, MHC class II, CD86, CD80, and CD83.

Autologous T cells were incubated with the matured antigen-loaded DCs at a ratio of 20×10⁴ T cells/well:4×10⁴ DCs/well or 4×10⁴ T cells/well:2×10⁴ DCs/well. The T cells were stimulated three times and T cell function evaluated 6 hr following the third stimulation by detection of the intracellular levels of the Th1 cytokines IFN-γ and TNF-α. In addition the extent of blast T cells was also assessed.

The measurement of the percentage of blast T cells in a T cell population can be used as a gauge of the extent of T cell proliferation. Blast T cells are defined as those cells possessing a higher FSC and SSC light scatter then the resting lymphocytes in the lymphocyte gate as assessed by flow cytometry. The percentage of T cell blast in the cultures after 14 days of culture is shown in FIG. 39. NS3 Chimigen™ Protein was efficient at inducing T cell proliferation (blast cell production), with the 2:1 T cell:DC ratio resulting in a lower background (buffer) T cell proliferative response compared with the 5:1 ratio. As a result, at the 2:1 T cell:DC ratio there was a marked difference in T cell proliferation upon stimulation with NS3 Chimigen™ Protein compared to buffer.

The IFN-γ response was measured at both the 5:1 and 2:1 T cell:DC ratios. The data is shown as the responses of each well of the group and as an average of the three wells with the standard deviation of the mean (FIG. 40). A comparison of the T cell IFN-γ response showed a marked difference between the 5:1 and 2:1 T cell:DC ratios. With the higher DC concentration there was little evidence of a vaccine candidate-induced IFN-γ response over that of control buffer. However with the lower DC concentration, very few T cells cultured with control buffer-loaded DCs produced IFN-γ whereas a high percentage of T cells cultured with vaccine candidate-loaded DCs produced IFN-γ. There was no reduction in IFN-γ producing cells with the T cells stimulated with DCs that had been loaded with 2.5 μg/mL compared with 5 Tg/mL of NS3 Chimigen™ Protein. The percentage of T cells expressing IFN-γ in the CD8+ and CD4+ population was quantified and is shown in FIG. 41. The percentage of CD8+ T cells expressing IFN-γ was comparable for T cells stimulated with 2.5 μg/mL of vaccine candidate-loaded DCs compared with TT-loaded DCs. Likewise, there was also a high percentage of CD4+ T cells that expressed IFN-γ upon stimulation with NS3 Chimigen™ Protein compared with control buffer. The percentage of CD4+ T cells expressing IFN-γ was comparable for T cells stimulated with vaccine candidate-loaded DCs compared with TT-loaded DCs. These results indicate that NS3 Chimigen™ Protein induces a marked IFN-γ response in both CD8+ and CD4+ T cell populations and suggests that the molecule is processed by the DCs in both the MHC class I and class II pathways.

FIG. 42 shows the percentage of T cells that have produced TNF-α as a result of a 6 hr stimulation with antigen-loaded mature DCs. These results are similar to the IFN-γ results. The TNF-α response was about equivalent or greater for the NS3 Chimigen™ Protein compared with TT. Stimulation with TT or NS3 Chimigen™ Protein-loaded DCs resulted in a higher percentage of CD4+ T cells expressing TNF-α compared with CD8+ T cells.

### NS3 Chimigen™ Protein Presentation by DCs Results in the Generation of CD8+ T Cells Expressing grB and pfn

The ability of T cells to produce the cytotoxic granular proteins grB and pfn was also assessed by ex vivo APAs. Immature DCs were loaded with control buffer, with TT (positive control), or varying concentrations of NS3 Chimigen™ Protein and upon maturation were incubated with autologous T cells. GrB and pfn expression were detected by intracellular staining using anti-grB and anti-pfn mAbs, respectively. FIG. 43 shows the percentage of CD8+ T cells that expressed grB and pfn following three stimulations with antigen-loaded mature DCs. NS3 Chimigen™ Protein-loaded DCs induced an increase in grB and pfn expression in CD8+ T cells compared to buffer control-treated DCs. These results indicated that NS3 Chimigen™ Vaccine induced the expression of grB and pfn in CD8+ T cells. This finding indicates the vaccine candidate is processed by the DCs in the MHC class I pathway for the effective presentation to T cells to result in their differentiation from naïve CD8+ T cells to cytotoxic T lymphocytes (CTLs).

### NS3 Chimigen™ Protein Presentation by Mature DCs Results in the Generation and Maintained Activation of CD8+ and CD4+ T Cells

T cells were stimulated with antigen-loaded DCs three times in an APA. After 6 d of culture following the third stimulation the T cells were harvested and investigated by flow cytometry for the percentage of blast cells as a measure of proliferation and for the expression of the activation marker CD69. In addition, as a means to estimate absolute numbers of T cells recovered from culture, the number of gated cells falling in the lymphocyte gate (R1 gate) based on FSC and SSC FFC analysis was determined.

The percentage of activated CD8+ and CD4+ T cells as assessed by CD69 expression is shown in FIG. 44. There was an increased percentage of both CD4+ and CD8+ T cells expressing CD69 in T cells stimulated with Chimigen3 Protein-loaded DCs compared with buffer control. There was a marked difference in the recovery of T cells from TT and Chimigen™ Protein stimulated wells compared with buffer control (FIG. 45). TT stimulated wells gave a higher T cell recovery than vaccine candidate stimulated wells. However the TT response is a recall response and thus the starting population of T cells reactive specific for TT would be expected to be higher than that of the starting population of naïve T cells specific for NS3. On examination of the T cell blasts present in the cultures, notably the percentage of blast cells/proliferating cells was higher in the NS3 Chimigen™ Protein-containing cultures compared to the TT cultures. There were very little blast cells/proliferating cells in the buffer control cultures. Thus, stimulation with the Chimigen3 Protein resulted in marked T cell activation and proliferation that is evident even six days following the third stimulation (day 20 of T cell culture). The NS3 Chimigen™ Protein is therefore very effective in the activation and expansion of both CD8+ and CD4+ T cells.

NS3 Chimigen™ Protein Presentation by mature DCs Induces the Generation of NS3-Specific CD8+ T Cells

To evaluate the specificity of the immune response to NS3 Chimigen™ Protein, the percentage of T cells specific to two immunodominant NS3 epitopes in the context of HLA-A2 was quantitated. This was determined by labeling T cells with NS3 peptide/HLA-A2 pentamers conjugated to PE. Naïve T cells were stimulated three times with DCs loaded with different concentrations of NS3 Chimigen™ Protein and compared to the respective control DCs loaded without antigen (buffer) in an APA. T cells were harvested six days after the third stimulation and NS3-specific T cells or EBV-specific T cells (control) were detected by tetramer labeling and analyzed by flow cytometry. One well of three of the buffer control group tested positive for EBV tetramer labeling (positive tetramer) in the CD8+ T cell population and no wells were positive for negative tetramer labeling (data not shown). As the T cells assessed were from the buffer control treated wells it would be expected that the number of EBV tetramer labeled T cells would be relatively low. The percentage of CD8+ T cells labeling with an NS3 pentamer following the APA is shown in FIG. 46. Loading DCs with NS3 Chimigen™ Protein resulted in the generation of T cells with specificity to NS3 epitopes. The marked expansion of CD8+ T cells with this specificity was apparent in four of six wells of the low DC concentration group. Thus the NS3 Chimigen™ Protein was able to induce the generation of T cells specific to NS3 immunodominant epitopes and it is probable that T cells with specificities to other NS3 epitopes were also present.

### Example 4

### Results with NS3-NS4B-NS5A Multiantigen Chimigen3 Protein

### Expression of HCV HCV NS3-NS4B-NS5A Chimigen™ Protein

Time course of the expression of HCV Multi-antigen Chimigen™ Protein in Sf9 cells was analyzed by Western blot after SDS-PAGE. By considering both factors of expression and degradation of HCV Multi-antigen Chimigen™ Protein, the best condition for protein expression was determined as MOI of 1.5 for 36 hours after infection.

Purification of HCV NS3-NS4B-NS5A Chimigen™ Protein from Clear Lysate of Sf9 Cell Pellet

### Ni-NTA Affinity Chromatography

As a first step of purification, HCV NS3-NS4B-NS5A Chimigen™ Protein was captured on a Ni-NTA Superflow3 column. The protein, bound on Ni-NTA Superflow3 column, was analyzed by SDS-PAGE and by Western blot. The Western blot showed a dominant band of the Chimigen™ Protein, however silver staining of the nitrocellulose membrane showed additional bands of non-immunoreactive proteins.

### HiTRap Q-XL Ion Exchange Chromatography

The proteins, captured by Ni-NTA Superflow column, were further separated by HiTrap Q XL 1 mL column chromatography. Protein was eluted in the flow-through fractions and the fractions eluted at salt concentration between 0.4 and 0.5 M NaCl. HCV NS3-NS4B-NS5A multi-antigen Chimigen™ Protein, bound on the column, had less contaminant than the protein in the flow-through fractions. At least 6 non-immunoreactive protein bands were seen by silver staining.

### Superdex 200 Chromatography

Proteins in the lysate were fractionated by a Superdex 200 column. The HCV NS3-NS4B-NS5A-multi-antigen Chimigen™ Protein was eluted in the first peak. Western blot and silver staining of the fraction were performed. The protein is shown as a dominant band on silver-staining; however numerous bands of contaminants were also visible. The results of western blot suggest the aggregation of the protein during purification

### Hydrophobic Interaction Chromatography on Phenyl-650C Toyopearl

Cell lysate containing the HCV NS3-NS4B-NS5A multi-antigen Chimigen™ Protein was loaded on Phenyl-650C Toyopearl® and eluted in both unabsorbed and absorbed fractions. Western blot and silver staining of the fractions were performed. In both fractions, HCV Multi-antigen Chimigen™ Protein was seen as a dominant band on Western blot and silver staining of the nitrocellulose membrane.

### Example 5

### Results with HCV Core Chimigen3 Protein

HCV Core Chimigen™ Protein has been Purified and Characterized

The HCV Core Chimigen3 Protein was purified by Ni chelation chromatography (Ni-NTA superflow) under denaturing conditions followed by cation exchange chromatography (CM sepharose). Purified protein was analyzed on 12% SDS-PAGE gels. The major band was the fusion protein (˜55 KDa), the second band noticed at 28 kDa is most likely a degradation product. After separation on a 12% SDS-PAGE gel, the purified proteins were electroblotted to nitrocellulose membranes. Western blotting was performed with anti-6×His-HRP conjugated antibody, anti-Fc specific-HRP conjugated antibody and anti-HCV core antibody with anti-Fab specific-HRP conjugated antibody as the secondary antibody. Bound antibodies were detected by chemiluminescence. Binding of the antibodies to the blot indicated that the purified HCV core-TBD ha an intact N-terminus, core and TBD portions. In addition, the lower molecular weight band was detected by all 3 antibodies, which indicated that it was a protein derived from the full length HCV core Chimigen™ molecule and was likely the result of degradation.

### HCV Core Chimigen™ Protein Binds to Immature DCs

HCV Core Chimigen™ vaccine was examined for its ability to bind to immature DCs. The cells were incubated in the presence and absence of various concentrations of HCV Core Chimigen™ Protein for 1 hr at 4° C. and binding was detected either by FFC or by confocal microscopy. For FFC analysis, bound HCV Core Chimigen™ Protein was detected with a biotinylated anti-mouse IgG1 mAb and SA-PE-Cy5.

The percentage of cells binding HCV Core Chimigen™ Protein (% positive cells) and the relative amount of bound Protein (MFI) was determined by FFC. With HCV Core Chimigen™ Protein at 5-40 μg/mL, approximately 100% of the cells were positive for binding, and there was a dose-dependent increase in the amount of bound HCV Core Chimigen™ Protein (FIG. 47). The high MFI of binding observed suggested that HCV Core Chimigen™ Protein binds very effectively and at high levels to immature DCs.

The binding of HCV Core Chimigen™ Protein was studied using confocal microscopy as well. The binding was detected with a FITC conjugated goat anti-mouse IgG. The blue fluorescent dye DAPI was used to image the nucleus. The confocal image and the corresponding light image showed that the protein binds to the membrane of immature DCs after a 1 hr pulse at 4° C.

### HCV Core Chimigen™ Protein Binds to Specific Receptors on Immature DCs

By virtue of the presence of Fc fragment, HCV Core Chimigen™ Protein is predicted to be able to bind via its TBD region to CD32 (FcγRII) on immature DCs. In addition, due to its mannose glycosylation, HCV Core Chimigen™ Protein is also predicted to bind to C-type lectin receptors such as CD206 (MMR). To determine the specificity of binding of HCV Core Chimigen™ Protein, immature DCs were incubated with HCV Core Chimigen™ Protein in the presence of blocking mAbs specific to CD32 or CD206. The binding was also examined in the presence of competing ligands, murine IgG Fc fragments for Fcγ receptors, and mannosylated BSA (mBSA) for C-type lectin receptors.

Immature DCs were incubated with PBS (buffer control), murine IgG Fc fragments (500 μg/mL), CD32 mAb (200 μg/mL), mannosylated BSA (500 μg/mL), or anti-CD206 (200 μg/mL) for 1 hr at 4° C. before incubation with HCV Core Chimigen™ Protein (30 μg/mL) for 1 hr at 4° C. The bound Protein was detected either with biotinylated anti-mouse IgG1 mAb or biotinylated anti-HCV core mAb followed by SA-PE-Cy5. The relative amount of bound HCV Core Chimigen™ Protein (MFI) was determined by FFC. The results from the binding and inhibition studies showed that HCV Core Chimigen™ Protein bound to Fcγ receptors such as CD32 and C-type lectin receptors such as CD206 (FIG. 48).

### HCV Core Chimigen™ Protein Presentation by DCs Results in Increased Intracellular IFN-γ Levels in CD8+ and CD4+ T Cells

The functional immune response to HCV Core Chimigen™ Protein was assessed by ex vivo antigen presentation assays. Immature DCs were loaded with PBS (buffer control), with tetanus toxoid (positive control), or varying concentrations of HCV Core Chimigen™ Protein. Upon maturation of the DCs, they were incubated with autologous T cells. T cell function was evaluated by detection of the intracellular levels of the Th1 cytokine IFN-γ. The CD3 and CD8 phenotype of the cells was also determined by FFC. FIGS. 49A and B show the percentage of CD8+ and CD4+ T cells, respectively, that express IFN-γ 12 hr following the third stimulation with antigen-loaded mature DCs. Tetanus toxoid was used as the positive control for effective antigen presentation. HCV Core Chimigen™ Protein-loaded DCs induced a marked increase in IFN-γ expression in CD8+ T cells compared to the no antigen control. There was an increase in the expression of IFN-γ in the CD4+ T cell population upon stimulation with HCV Core Chimigen™ Protein-loaded DCs. These results indicate that HCV Core Chimigen™ Protein induces an IFN-γ response in both CD8+ and CD4+ T cell populations and suggests that the molecule is processed by the DCs in both the MHC class I and class II pathways.

### HCV Core Chimigen™ Protein Presentation by Mature DCs Induces the Generation of HCV Core-Specific CD8+ T Cells

To evaluate the specificity of the immune response to HCV core, the percentage of T cells specific to an immunodominant HCV core epitope in the context of HLA-B7 was quantitated. This was achieved by labeling T cells with an HCV core peptide/HLA-137 tetramer conjugated to PE. In addition, T cells were labeled with CD4 and CD8 specific mAbs.

HCV core naïve T cells were stimulated three times with DCs loaded with different concentrations of HCV Core Chimigen™ Protein and compared to the respective control DCs loaded with no antigen, with tetanus toxoid, or with TBD. T cells were harvested 5 days after the third stimulation and HCV core-specific T cells detected to by two-dimensional FFC. The two dimensional FFC dot plot in FIG. 50 shows that T cells incubated with DCs loaded with HCV Core Chimigen™ Protein showed a small increase in the core tetramer positive T cells.

The disclosure of U.S. Provisional Application No. 60/726,701, including all Attachments, is incorporated herein by reference in its entirety.

All publications, patent applications, and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A chimeric antigen for eliciting an immune response, said chimeric antigen comprising an immune response domain and a target binding domain, wherein the immune response domain comprises a hepatitis C (HCV) antigen and the target binding domain comprises an antibody fragment.

2. The chimeric antigen of claim 1, wherein the antibody fragment is xenotypic antibody fragment.

3. The chimeric antigen of claim 1, wherein the chimeric antigen elicits a humoral immune response, a cellular immune response, or a both humoral immune response and a cellular immune response.

4. The chimeric antigen of claim 1, wherein the chimeric antigen elicits a Th1 immune response, a Th2 immune response or both a Th1 and a Th2 immune response.

5. The chimeric antigen of claim 1, wherein the immune response is an in vivo immune response.

6. The chimeric antigen of claim 1, wherein the immune response domain comprises more than one protein.

7. The chimeric antigen of claim 1, wherein the immune response domain comprises one or more immunogenic portions of one or more proteins selected from the group consisting of a HCV Core (1-191) protein, a HCV Core (1-177) protein, a HCV p7 protein, a HCV E1 protein, a HCV E2 protein, a HCV E1-E2 protein, a HCV NS3 protein, a HCV NS4B protein, and a HCV NS5A protein.

8. The chimeric antigen of claim 1, wherein the target binding domain is capable of binding to an antigen presenting cell (APC).

9. The chimeric antigen of claim 2, wherein the antibody fragment is a Fc fragment.

10. The chimeric antigen of claim 1, further comprising one or more of a 6×His tag, a protease cleavage site, and a linker for linking the immune response domain and the target binding domain.

11. The chimeric antigen of claim 10, wherein the linker is selected from the group consisting of leucine zippers, biotin bound to avidin, and a covalent peptide linkage.

12. The chimeric antigen of claim 1, wherein the chimeric antigen is glycosylated.

13. The chimeric antigen of claim 1, wherein the chimeric antigen is mannose glycosylated.

14. The chimeric antigen of claim 1, wherein the antibody fragment comprises an immunoglobulin heavy chain fragment.

15. The chimeric antigen of claim 14, wherein the immunoglobulin heavy chain fragment comprises a hinge region.

16. The chimeric antigen of claim 14, wherein the immunoglobulin heavy chain fragment comprises all or a part of an antibody fragment selected from the group consisting of the C_{H}1, the hinge region, the C_{H}2 domain, and the C_{H}3 domain.

17. A method of delivering an antigen to an antigen presenting cell, the method comprising administering to the antigen presenting cell a chimeric antigen of claim 1.

18. The method of claim 17, wherein the antigen presenting cell is a dendritic cell.

19. A method of activating an antigen presenting cell, the method comprising contacting the antigen presenting cell with a chimeric antigen of claim 1.

20. The method of claim 19, wherein the contacting takes place ex vivo.

21. The method of claim 19, wherein the contacting takes places in vivo.

22. The method of claim 21, wherein the contacting takes place in a human.

23. The method of claim 21, wherein the method comprises administering to a subject a composition comprising a chimeric antigen comprising an immune response domain and a target binding domain, wherein the immune response domain comprises a hepatitis C(HCV) antigen and the target binding domain comprises an antibody fragment, and wherein the antigen presenting cell is in the subject.

24. The method of claim 20, wherein the contacting results in a humoral immune response, a cellular immune response, or both a humoral immune response and a cellular immune response.

25. The method of claim 24 wherein the cellular immune response is one or more of a Th1 response, a Th2 response, and a CTL response.

26. The method of claim 23, wherein the subject has, or is likely to have, an immune-treatable condition.

27. The method of claim 26, wherein the immune-treatable condition is an acute infection.

28. The method of claim 26, wherein the immune-treatable condition is a chronic infection.

29. The method of claim 28, wherein the chronic infection is a chronic hepatitis C viral infection.

30. The method of claim 26, wherein the immune-treatable condition is a hepatitis C viral infection and the immune response domain comprises one or more antigenic portions of one or more proteins selected from the group consisting of a HCV Core (1-191) protein, a HCV Core (1-177) protein, a HCV E1 protein, a HCV E2 protein, a HCV E1-E2 protein, a HCV P7 protein, a HCV NS3 protein, a HCV NS4B protein, and a HCV NS5 A protein.

31. The method of claim 23, wherein the subject is vaccinated against a viral infection.

32. The method of claim 23, wherein the subject is prophylactically vaccinated against a viral infection.

33. The method of claim 31, wherein the subject is therapeutically vaccinated against an existing viral infection.

34. A method of producing a chimeric antigen comprising:
(a) providing a microorganism or a cell, the microorganism or cell comprising a polynucleotide that encodes a chimeric antigen; and
(b) culturing said microorganism or cell under conditions whereby the chimeric antigen is expressed.

35. The method of claim 34, wherein the microorganism or cell is a eukaryotic microorganism or cell.

36. The method of claim 34, wherein the cell is a yeast cell, a plant cell or an insect cell.

37. The method of claim 34, wherein the chimeric antigen is post-translationally modified to comprise glycosylation.

38. The method of claim 34, wherein the chimeric antigen is post-translationally modified to comprise a mannose glycosylation.

39. A polynucleotide encoding a chimeric antigen, said polynucleotide comprising a first polynucleotide portion encoding an immune response domain and a second polynucleotide portion encoding a target binding domain, wherein the target binding domain comprises an antibody fragment.

40. The polynucleotide of claim 39, wherein the antibody fragment is a xenotypic antibody fragment.

41. The polynucleotide of claim 39, wherein the polynucleotide comprises a nucleotide sequence selected from the group consisting of the nucleotide sequences set forth in SEQ ED NOs:39 and 41-51.

42. The polynucleotide of claim 39, wherein the polynucleotide encodes a chimeric antigen that is at least 90% identical to an entire amino acid sequence selected from the group consisting of the amino acid sequences set forth in SEQ ID NOs:40 and 52-62.

43. The polynucleotide of claim 39, wherein the polynucleotide selectively hybridizes under stringent conditions to a polynucleotide having a nucleotide sequence selected from the group consisting of nucleotide sequences set forth in SEQ ID NOs:39 and 41-51.

44. A vector comprising the polynucleotide of claim 39.

45. The vector of claim 44, wherein the polynucleotide is operably linked to a transcriptional regulatory element (TRE).

46. A microorganism or cell comprising the polynucleotide of claim 39.

47. An article of manufacture comprising a chimeric antigen of claim 1 and instructions for administering the chimeric antigen to a subject in need thereof.

48. A pharmaceutical composition comprising a chimeric antigen of claim 1 and a pharmaceutically acceptable excipient.

49. A method of producing a chimeric antigen comprising:
(a) providing a microorganism or a cell, the microorganism or cell comprising a polynucleotide that encodes a target binding domain-linker molecule, wherein the target binding domain-linker molecule comprises a target binding domain bound to a linker molecule;
(b) culturing said microorganism or cell under conditions whereby the target binding domain-linker molecule is expressed; and
(c) contacting the target binding domain-linker molecule and an immune response domain under conditions that allow for the binding of the linker to the immune response domain, the binding resulting in a chimeric antigen.


SEQUENCE LISTING (62 sequences)

SEQ ID NO: 1 - protein, 8 aa, Artificial
  exemplary peptide linker
SRPQGGGS

SEQ ID NO: 2 - protein, 5 aa, Mus musculus
VDKKI

SEQ ID NO: 3 - protein, 9 aa, Artificial
  synthetic polymer
VLSDFKTWL

SEQ ID NO: 4 - protein, 9 aa, Artificial
  synthetic polymer
CINGVCWTV

SEQ ID NO: 5 - protein, 9 aa, Artificial
  synthetic polymer
GLCTLVAML

SEQ ID NO: 6 - protein, 10 aa, Artificial
  synthetic polymer
KLVALGINAV

SEQ ID NO: 7 - DNA, 55 bp, Artificial
  PCR primer
tgtcattctgcggccgcaaggcggcgggatccgtggacaagaaaattgtgccagg

SEQ ID NO: 8 - DNA, 36 bp, Artificial
  PCR primer
acgaatcaagctttgcagcccaggagagtgggagag

SEQ ID NO: 9 - DNA, 870 bp, Artificial
  synthetic construct
atgtcgtactaccatcaccatcaccatcacgattacgatatcccaacgaccgaaaacctgtattttcagggcgccatggatccggaattcaaaggcctacgtcgacgagctcaactagtgcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 10 - protein, 289 aa, Artificial
  Synthetic Construct
MSYYHHHHHHDYDIPTTENLYFQGAMDPEFKGLRRRAQLVRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS

SEQ ID NO: 11 - DNA, 92 bp, Artificial
  synthetic oligonucleotide
gcatggtccatggtaagcgctattgttttatatgtgcttttggcggcggcggcgcattctgcctttgcggatctgcaggtacggtccgatgc

SEQ ID NO: 12 - DNA, 92 bp, Artificial
  synthetic oligonucleotide
gcatcggaccgtacctgcagatccgcaaaggcagaatgcgccgccgccgccaaaagcacatataaaacaatagcgcttaccatggaccatgc

SEQ ID NO: 13 - DNA, 27 bp, Artificial
  PCR primer
ccggaattctccggttcctggctaagg

SEQ ID NO: 14 - DNA, 28 bp, Artificial
  PCR primer
ggactagtccgcacacgacatcttccgt

SEQ ID NO: 15 - DNA, 33 bp, Artificial
  PCR primer
gtttctaacgcgtcgtactaccatcaccatcac

SEQ ID NO: 16 - DNA, 34 bp, Artificial
  PCR primer
ccggggtaccttacagcccaggagagtgggagag

SEQ ID NO: 17 - DNA, 21 bp, Artificial
  PCR primer
ctggtagttcttcggagtgtg

SEQ ID NO: 18 - DNA, 32 bp, Artificial
  PCR primer
ggtagtacgacgcgttagaaacggcgaccaac

SEQ ID NO: 19 - DNA, 26 bp, Artificial
  PCR primer
ccggaattcgcgcccatcacggcgta

SEQ ID NO: 20 - DNA, 33 bp, Artificial
  PCR primer
ccggactagtccggccgacatgcatgtcatgat

SEQ ID NO: 21 - DNA, 33 bp, Artificial
  PCR primer
ctgccagtcctgcccgcgcgttgagtcctggag

SEQ ID NO: 22 - DNA, 29 bp, Artificial
  PCR primer
ggcaggactggcagggggaagccaggcat

SEQ ID NO: 23 - DNA, 32 bp, Artificial
  PCR primer
gcgcactagtgtctcagcacttaccgtacatc

SEQ ID NO: 24 - DNA, 34 bp, Artificial
  PCR primer
cggcgcggccgcccgcagcacacgacatcttccg

SEQ ID NO: 25 - DNA, 34 bp, Artificial
  PCR primer
caacagcggaccccccgcggagcctttcaagtag

SEQ ID NO: 26 - DNA, 27 bp, Artificial
  PCR primer
gtccgctgttgtgccccgcgggacacg

SEQ ID NO: 27 - DNA, 29 bp, Artificial
  PCR primer
ccggactagtccggccgacatgcatgtca

SEQ ID NO: 28 - DNA, 33 bp, Artificial
  PCR primer
gagggactagtgtccggttcctggctaagggac

SEQ ID NO: 29 - DNA, 35 bp, Artificial
  PCR primer
ccggtctagattatgatcctctagtacttctcgac

SEQ ID NO: 30 - DNA, 27 bp, Artificial
  PCR primer
cggaattcatgagcacgaatcctaaac

SEQ ID NO: 31 - DNA, 27 bp, Artificial
  PCR primer
ggactagtccgaagatagagaaagagc

SEQ ID NO: 32 - DNA, 34 bp, Artificial
  PCR primer
agtaagatctttacagcccaggagagtgggagag

SEQ ID NO: 33 - DNA, 28 bp, Artificial
  PCR primer
ccggaattctaccaagtgcgcaattcct

SEQ ID NO: 34 - DNA, 31 bp, Artificial
  PCR primer
gcgcactagtcccttcgcccagttccccacc

SEQ ID NO: 35 - DNA, 33 bp, Artificial
  PCR primer
gcgcactagtcacccacgtcaccgggggaaatg

SEQ ID NO: 36 - DNA, 32 bp, Artificial
  PCR primer
gcgcgcggccgcccgtactcccacttaatggc

SEQ ID NO: 37 - protein, 19 aa, Autographa californica
MPLYKLLNVLWLVAVSNAI

SEQ ID NO: 38 - DNA, 57 bp, Autographa californica
atgcccttgtacaaattgttaaacgttttgtggttggtcgccgtttctaacgcgatt

SEQ ID NO: 39 - DNA, 2190 bp, Artificial
  synthetic construct
atgtcgtactaccatcaccatcaccatcacgattacgatatcccaacgaccgaaaacctgtattttcagggcgccatggatccggaattctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgcggactagtgcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagtttagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 40 - protein, 729 aa, Artificial
  Synthetic Construct
MSYYHHHHHHDYDIPTTENLYFQGAMDPEFSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCGLVRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS

SEQ ID NO: 41 - DNA, 2274 bp, Artificial
  synthetic construct
atggtaagcgctattgttttatatgtgcttttggcggcggcggcgcattctgcctttgcgtatctgcaggtacggtccgaaaccatgtcgtactaccatcaccatcaccatcacgattacgatatcccaacgaccgaaaacctgtattttcagggcgccatggatccggaattctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgcggactagtgcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagtttagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 42 - DNA, 2211 bp, Artificial
  synthetic construct
atgcccttgtacaaattgttaaacgttttgtggttggtcgccgtttctaacgcgtcgtactaccatcaccatcaccatcacgattacgatatcccaacgaccgaaaacctgtattttcagggcgccatggatccggaattctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgcggactagtgcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgtaa

SEQ ID NO: 43 - DNA, 2808 bp, Artificial
  synthetic construct
atggtaagcgctattgttttatatgtgcttttggcggcggcggcgcattctgcctttgcgtatctgcaggtacggtccgaaaccatgtcgtactaccatcaccatcaccatcacgattacgatatcccaacgaccgaaaacctgtattttcagggcgccatggatccggaattcgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgcaggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccccactagtgcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 44 - DNA, 2724 bp, Artificial
  synthetic construct
atgtcgtactaccatcaccatcaccatcacgattacgatatcccaacgaccgaaaacctgtattttcagggcgccatggatccggaattcgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctcccggactcaacgcgcgggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccccactagtgcggccgcaaggcggcggatccgtggacaagaaaattgtgcccagggattgtggttgtaagccttgcatatgtacagtcccagaagtatcatctgtcttcatcttccccggaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 45 - DNA, 2808 bp, Artificial
  synthetic construct
atggtaagcgctattgttttatatgtgcttttggcggcggcggcgcattctgcctttgcgtatctgcaggtacggtccgaaaccatgtcgtactaccatcaccatcaccatcacgattacgatatcccaacgaccgaaaacctgtattttcagggcgccatggatccggaattcgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctcccggactcaacgcgcgggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccccactagtgcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 46 - DNA, 4938 bp, Artificial
  synthetic construct
atggtaagcgctattgttttatatgtgcttttggcggcggcggcgcattctgcctttgcgtatctgcaggtacggtccgaaaccatgtcgtactaccatcaccatcaccatcacgattacgatatcccaacgaccgaaaacctgtattttcagggcgccatggatccggaattcgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctccgcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgcgcgggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccggactagtgtctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgcgggcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 47 - DNA, 4155 bp, Artificial
  synthetic construct
atggtaagcgctattgttttatatgtgcttttggcggcggcggcgcattctgcctttgcgtatctgcaggtacggtccgaaaccatgtcgtactaccatcaccatcaccatcacgattacgatatcccaacgaccgaaaacctgtattttcagggcgccatggatccggaattcgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctccgcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgcgcgggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccggactagtgtccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgcgggcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 48 - DNA, 1380 bp, Artificial
  synthetic construct
atgtcgtactaccatcaccatcaccatcacgattacgatatcccaacgaccgaaaacctgtattttcagggcgccatggatccggaattcatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttcggactagtgcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 49 - DNA, 1428 bp, Artificial
  synthetic construct
atgtcgtactaccatcaccatcaccatcacgattacgatatcccaacgaccgaaaacctgtattttcagggcgccatggatccggaattctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaggactagtgcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 50 - DNA, 1938 bp, Artificial
  synthetic construct
atgtcgtactaccatcaccatcaccatcacgattacgatatcccaacgaccgaaaacctgtattttcagggcgccatggatccggaattcacccacgtcaccgggggaaatgccggccgcaccacggctgggcttgttggtctccttacaccaggcgccaagcagaacatccaactgatcaacaccaacggcagttggcacatcaatagcacggccttgaattgcaatgaaagccttaacaccggctggttagcagggctcttctatcaacacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcggctggactagtgcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 51 - DNA, 2517 bp, Artificial
  synthetic construct
atgtcgtactaccatcaccatcaccatcacgattacgatatcccaacgaccgaaaacctgtattttcagggcgccatggatccggaattctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaaatgccggccgcaccacggctgggcttgttggtctccttacaccaggcgccaagcagaacatccaactgatcaacaccaacggcagttggcacatcaatagcacggccttgaattgcaatgaaagccttaacaccggctggttagcagggctcttctatcaacacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcggctggactagtgcggccgcaaggcggcggatccgtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaaagcttgtcgagaagtactagaggatcataa

SEQ ID NO: 52 - protein, 757 aa, Artificial
  Synthetic Construct
MVSAIVLYVLLAAAAHSAFAYLQVRSETMSYYHHHHHHDYDIPTTENLYFQGAMDPEFSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCGLVRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS

SEQ ID NO: 53 - protein, 736 aa, Artificial
  Synthetic Construct
MPLYKLLNVLWLVAVSNASYYHHHHHHDYDIPTTENLYFQGAMDPEFSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCGLVRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGL

SEQ ID NO: 54 - protein, 935 aa, Artificial
  Synthetic Construct
MVSAIVLYVLLAAAAHSAFAYLQVRSETMSYYHHHHHHDYDIPTTENLYFQGAMDPEFAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSAPLVRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS

SEQ ID NO: 55 - protein, 907 aa, Artificial
  Synthetic Construct
MSYYHHHHHHDYDIPTTENLYFQGAMDPEFAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRAGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSAPLVRPQGGGSVDKKIVPRDCGCKPCICTVPEVSSVFIFPGKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS

SEQ ID NO: 56 - protein, 935 aa, Artificial
  Synthetic Construct
MVSAIVLYVLLAAAAHSAFAYLQVRSETMSYYHHHHHHDYDIPTTENLYFQGAMDPEFAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRAGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSAPLVRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS

SEQ ID NO: 57 - protein, 1645 aa, Artificial
  Synthetic Construct
MVSAIVLYVLLAAAAHSAFAYLQVRSETMSYYHHHHHHDYDIPTTENLYFQGAMDPEFAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSAGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRAGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSAGLVSQHLPYIEQGMMLAEQFKQKALGLLQTASRHAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCGRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS

SEQ ID NO: 58 - protein, 1384 aa, Artificial
  Synthetic Construct
MVSAIVLYVLLAAAAHSAFAYLQVRSETMSYYHHHHHHDYDIPTTENLYFQGAMDPEFAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSAGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRAGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSAGLVSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCGRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS

SEQ ID NO: 59 - protein, 459 aa, Artificial
  Synthetic Construct
MSYYHHHHHHDYDIPTTENLYFQGAMDPEFMSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFGLVRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS

SEQ ID NO: 60 - protein, 475 aa, Artificial
  Synthetic Construct
MSYYHHHHHHDYDIPTTENLYFQGAMDPEFYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAEGLVRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS

SEQ ID NO: 61 - protein, 645 aa, Artificial
  Synthetic Construct
MSYYHHHHHHDYDIPTTENLYFQGAMDPEFTHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAAGLVRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS

SEQ ID NO: 62 - protein, 838 aa, Artificial
  Synthetic Construct
MSYYHHHHHHDYDIPTTENLYFQGAMDPEFYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAAGLVRPQGGGSVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQSLSRSTRGS